(19) United States
(12) Patent Application Publication
(10) Pub. No.: US 2024/0092757 A1
(43) Pub. Date: Mar. 21, 2024

(54) HETEROARYL DIAMIDE IRE1/XBP1s ACTIVATORS

(71) Applicants: Protego Biopharma, Inc., San Diego, CA (US); The Scripps Research Institute, La Jolla, CA (US)

(72) Inventors: Richard F. Labaudiniere, Charleston, SC (US); Bradley Dean Tait, North Andover, MA (US); Hank Michael James Petrassi, La Jolla, CA (US); Jeffery W. Kelly, La Jolla, CA (US); R. Luke Wiseman, Chula Vista, CA (US); Kyunga Lee, La Jolla, CA (US); Adrian Marco Guerrero, La Jolla, CA (US)

(21) Appl. No.: 18/504,724

(22) Filed: Nov. 8, 2023

Related U.S. Application Data
(63) Continuation of application No. PCT/US2022/031186, filed on May 26, 2022.
(60) Provisional application No. 63/202,118, filed on May 27, 2021.

(51) Int. Cl. C07D 401/12; C07D 231/38; C07D 233/88; C07D 403/12

(52) U.S. Cl. CPC C07D 401/12; C07D 231/38; C07D 233/88; C07D 403/12

(57) ABSTRACT

Provided herein are compounds that activate IRE1/XBP1s, and pharmaceutically acceptable derivatives thereof. Also provided are pharmaceutical compositions containing the compounds and methods of using the compounds for treating a subject having a disease or disorder that may be ameliorated by increasing IRE1/XBP1s activity.

### RELATED APPLICATIONS

This application is a continuation of International PCT Application No. PCT/US2022/031186, filed May 26,2022, which claims benefit of priority to U.S. provisional application No. 63/202,118, filed May 27, 2021. The contents of the above-referenced applications are incorporated herein by reference in their entirety.

### FIELD

Provided herein are compounds that are IRE1/XBP1s activators. In one embodiment, the compounds are heteroaryl diamides.

### BACKGROUND

The unfolded protein response (UPR) is the primary signaling pathway activated in response to endoplasmic reticulum (ER) stress. The UPR is comprised of three signaling cascades activated downstream of the ER stress-sensing proteins IRE1 (inositol requiring enzyme 1), PERK (protein kinase RNA-like endoplasmic reticulum kinase), and ATF6 (activating transcription factor 6). In response to acute ER stress, activation of these pathways results in transcriptional and translational remodeling to alleviate the misfolded protein load in the ER lumen and promote adaptive remodeling of ER function and global cellular physiology. However, in response to chronic or severe ER insults, prolonged UPR signaling can induce a pro-apoptotic response that results in cellular death. Thus, through this combination of adaptive and pro-apoptotic signaling, the UPR functions at a critical intersection in dictating cellular function and survival in response to diverse pathologic insults that induce ER stress.

The capacity for UPR signaling to promote adaptive remodeling of ER function makes the three UPR signaling pathways attractive targets to ameliorate pathologic imbalances in ER proteostasis implicated in etiologically diverse diseases. The IRE1 pathway is the most evolutionarily conserved arm of the UPR. It is found in organisms ranging from yeast to mammals. IRE1 is an ER transmembrane protein that is activated in response to ER stress through a mechanism involving autophosphorylation and oligomerization. This response leads to the activation of the cytosolic endoribonuclease (RNAse) domain of IRE1 that is involved in the non-canonical splicing of the X-box binding protein 1 (XBP1) mRNA. IRE1-dependent XBP1 splicing produces an mRNA frameshift that leads to the translation of the active spliced XBP1 (or XBP1s) bZIP transcription factor. Upon activation, XBP1s transcriptionally regulates the expression of multiple stress-responsive genes involved in diverse biological functions including ER proteostasis maintenance and lipid homeostasis.

Apart from XBP1 splicing, the activated IRE1 endoribonuclease domain can also promote the degradation of ER-localized mRNAs through a process referred to as regulated IRE1-dependent decay (or RIDD). While the functional implications of this IRE1 activity remain to be fully established, recent results show that RIDD serves a protective role through the selective degradation of mRNA encoding the pro-apoptotic factors (e.g., DR5) and promotion of microautophagy through the degradation of BLOS1 mRNA.

The role of IRE1/XBP1s in these biological processes implicates the importance of same as therapeutic targets. Thus, there is a need for compounds and compositions that activate IRE1/XBP1s.

### SUMMARY

Provided herein are compounds and pharmaceutical compositions containing the compounds that activate IRE1/XBP1s. In certain embodiments, the compounds provided herein possess improved metabolic stabilities and/or PK and/or PD properties, as compared to compounds known in the art. See, e.g., PCT Patent Application Publication No. WO 2021/007594. In one embodiment, the compounds for use in the compositions and methods provided herein have Formula I:

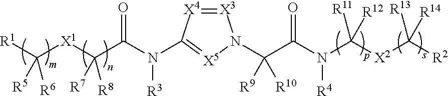

or a pharmaceutically acceptable derivative thereof, wherein X¹-X⁵, R¹-R¹⁴, m, n, p and s are as defined herein.

Also provided herein are methods of activating IRE1 and/or XBP1s by contacting IRE1 with a compound or composition provided herein. In another embodiment, provided herein are methods of enhancing IRE1 signaling by contacting an IRE1 expressing cell with a compound or composition provided herein. In another embodiment, provided herein are methods of treating diseases that may be treated by enhancing IRE1 signaling or by activating IRE1 and/or XBP1s. In certain embodiments, such diseases include cardiovascular diseases, neurodegenerative diseases, metabolic disorders, hepatic disorders, protein misfolding disorders and gastrointestinal disorders.

### DETAILED DESCRIPTION

### I. DEFINITIONS

To facilitate understanding of the disclosure set forth herein, a number of terms are defined below.

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

The singular forms “a,” “an,” and “the” include plural references, unless the context clearly dictates otherwise.

As used herein “subject” is an animal, such as a mammal, including human, such as a patient.

As used herein, biological activity refers to the in vivo activities of a compound or physiological responses that result upon in vivo administration of a compound, composition or other mixture. Biological activity, thus, encompasses therapeutic effects and pharmacokinetic behavior of such compounds, compositions and mixtures. Biological activities can be observed in in vitro systems designed to test for such activities.

As used herein, pharmaceutically acceptable derivatives of a compound include, but are not limited to, salts, esters, enol ethers, enol esters, acetals, ketals, orthoesters, hemiacetals, hemiketals, acids, bases, clathrates, solvates or hydrates thereof. Such derivatives may be readily prepared by those of skill in this art using known methods for such derivatization. The compounds produced may be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and inorganic salts, such as but not limited to, sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates, mesylates, and fumarates. Pharmaceutically acceptable esters include, but are not limited to, alkyl, alkenyl, alkynyl, aryl, aralkyl, and cycloalkyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is alkyl, alkenyl, alkynyl, aryl, aralkyl and cycloalkyl. Pharmaceutically acceptable enol esters include, but are not limited to, derivatives of formula C═C(OC(O)R) where R is hydrogen, alkyl, alkenyl, alkynyl, aryl, aralkyl and cycloalkyl. Pharmaceutically acceptable solvates and hydrates are complexes of a compound with one or more solvent or water molecules, or 1 to about 100, or 1 to about 10, or one to about 2, 3 or 4, solvent or water molecules.

As used herein, treatment means any manner in which one or more of the symptoms of a disease or disorder are ameliorated or otherwise beneficially altered. Treatment also encompasses any pharmaceutical use of the compositions herein, such as use for treating diseases that may be treated by enhancing IRE1 signaling or by activating IRE1 and/or XBP1s.

As used herein, amelioration of the symptoms of a particular disorder by administration of a particular compound or pharmaceutical composition refers to any lessening, whether permanent or temporary, lasting or transient that can be attributed to or associated with administration of the compound or pharmaceutical composition.

As used herein, and unless otherwise indicated, the terms “manage,” “managing” and “management” encompass preventing the recurrence of the specified disease or disorder in a subject who has already suffered from the disease or disorder, and/or lengthening the time that a subject who has suffered from the disease or disorder remains in remission. The terms encompass modulating the threshold, development and/or duration of the disease or disorder, or changing the way that a subject responds to the disease or disorder.

As used herein, the IC₅₀ refers to an amount, concentration or dosage of a particular test compound that achieves a 50% inhibition of a maximal response in an assay that measures such response.

As used herein, the EC₅₀ refers to an amount, concentration or dosage of a particular test compound that achieves a 50% effect of a maximal response in an assay that measures such response.

As used herein, the K_{d} refers to the measured equilibrium dissociation constant between a compound (or ligand) and a protein (or binding domain of a protein).

As used herein, “PK” is the pharmacokinetics in an animal, such as a mammal, including a human, such as a patient.

As used herein, “PD” is the pharmacodynamics in an animal, such as a mammal, including a human, such as a patient.

Where moieties are specified by their conventional chemical formulae, written from left to right, they equally encompass the chemically identical moieties that would result from writing the structure from right to left, e.g., —CH₂O— is equivalent to —OCH₂—

The term “alkyl,” by itself or as part of another substituent, means, unless otherwise stated, a straight (i.e., unbranched) or branched chain saturated hydrocarbon radical, which can include di- and multivalent radicals, having the number of carbon atoms designated (i.e., C₁-C₁₀ means one to ten carbons). Examples of alkyl groups include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like.

The term “alkenyl,” by itself or as part of another substituent, means, unless otherwise stated, a straight (i.e., unbranched) or branched chain hydrocarbon radical having one or more carbon-carbon double bonds, which can include di- and multivalent radicals, having the number of carbon atoms designated (i.e., C₁-C₁₀ means one to ten carbons). Examples of alkenyl groups include, but are not limited to, vinyl (i.e., ethenyl), 2-propenyl, crotyl, 2-isopentenyl, 2-(butadienyl), 2,4-pentadienyl, 3-(1,4-pentadienyl), and the higher homologs and isomers.

The term “alkynyl,” by itself or as part of another substituent, means, unless otherwise stated, a straight (i.e., unbranched) or branched chain hydrocarbon radical having one or more carbon-carbon triple bonds, which can include di- and multivalent radicals, having the number of carbon atoms designated (i.e., C₁-C₁₀ means one to ten carbons). Examples of alkynyl groups include, but are not limited to, ethynyl, 1- and 3-propynyl, 3-butynyl, and the higher homologs and isomers.

The term “alkylene” by itself or as part of another substituent means a divalent radical derived from an alkyl, as exemplified, but not limited, by —CH₂CH₂CH₂CH₂—. Typically, an alkyl (or alkylene) group will have from 1 to 24 carbon atoms, including those groups having 10 or fewer carbon atoms. A “lower alkyl” or “lower alkylene” is a shorter chain alkyl or alkylene group, generally having six or fewer carbon atoms.

The terms “alkoxy,” “alkylamino,” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a straight or branched chain hydrocarbon radical, consisting of a heteroatom selected from the group consisting of O, N, P, Si and S, and wherein the nitrogen and sulfur atoms may optionally be oxidized and the nitrogen atom may have an alkyl substituent to fulfill valency and/or may optionally be quaternized. The heteroatom(s) O, N, P, Si and S may be placed at any interior position of the heteroalkyl group. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For alkylene and heteroalkylene linking groups, no orientation of the linking group is implied by the direction in which the formula of the linking group is written. For example, the formula —C(O)₂R′— represents both —C(O)₂R′— and —R′C(O)₂—

The terms “cycloalkyl” and “heterocycloalkyl”, by themselves or in combination with other terms, represent, unless otherwise stated, cyclic versions of “alkyl” and “heteroalkyl”, respectively, including bicyclic, tricyclic and bridged bicyclic groups. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, norbornanyl, bicyclo[2.2.2]octanyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, 1- or 2-azabicyclo[2.2.2]octanyl, and the like.

The terms “halo,” by itself or as part of another substituent, means, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is meant to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent which can be a single ring or multiple rings (in one embodiment from 1 to 3 rings) which are fused together or linked covalently. The term “heteroaryl” refers to aryl groups that contain from one to four heteroatoms selected from N, O, and S in the ring(s), wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituent moieties for aryl and heteroaryl ring systems may be selected from the group of acceptable substituent moieties described herein. The term “heteroarylium” refers to a heteroaryl group that is positively charged on one or more of the heteroatoms.

The term “oxo” as used herein means an oxygen atom that is double bonded to a carbon atom.

Each of the above terms (e.g., “alkyl,” “heteroalkyl,” “aryl” and “heteroaryl”) are meant to include both substituted and unsubstituted forms of the indicated radical. Non-limiting examples of substituent moieties for each type of radical are provided below.

Substituent moieties for alkyl, heteroalkyl, alkylene, alkenyl, heteroalkylene, heteroalkenyl, alkynyl, cycloalkyl, heterocycloalkyl, cycloalkenyl, and heterocycloalkenyl groups are, in one embodiment, selected from, deuterium, —OR′, ═O, NR′, ═N—OR′, —NR′R″, —SR′, halo, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR—C(NR′R″R′″)═NR″′, —NR—C(NR′R″)═NR′″, —S(O)R′, —S(O)₂R′, —S(O)₂NR′R″, —NRSO₂R′, —NRSO₂NR′R″, —CN and —NO₂ in a number ranging from zero to the number of hydrogen atoms in such radical. In one embodiment, substituent moieties for cycloalkyl, heterocycloalkyl, cycloalkenyl, and heterocycloalkenyl groups also include substituted and unsubstituted alkyl, substituted and unsubstituted alkenyl, and substituted and unsubstituted alkynyl. R′, R″, R″′ and R″″ each in one embodiment independently are hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. When a compound provided herein includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituent moieties, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Substituent moieties for aryl and heteroaryl groups are, in one embodiment, selected from deuterium, halo, substituted and unsubstituted alkyl, substituted and unsubstituted alkenyl, and substituted and unsubstituted alkynyl, —OR′, —NR′R″, —SR′, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of hydrogens on the aromatic ring system; and where R′, R″, R″′ and R″″ are, in one embodiment, independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound provided herein includes more than one R group, for example, each of the R groups is independently selected as are each R′, R″, R′″ and R″″ groups when more than one of these groups is present.

Two of the substituent moieties on adjacent atoms of an aryl or heteroaryl ring may optionally form a ring of the formula -Q′-C(O)—(CRR′)_{q}-Q″-, wherein Q′ and Q″ are independently —NR—, —O—, —CRR′— or a single bond, and q is an integer of from 0 to 3. Alternatively, two of the substituent moieties on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituent moieties on adjacent atoms of the aryl or heteroaryl ring may optionally be replaced with a substituent of the formula —(CRR′)ₛ—X′—(CR″R″′)_{d}—, where s and d are independently integers of from 0 to 3, and X′ is —O—, —NR′—, —S—, —S(O)—, —S(O)₂—, or —S(O)₂NR′—. The substituent moieties R, R′, R″ and R′″ are, in one embodiment, independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “heteroatom” or “ring heteroatom” is meant to include oxygen (O), nitrogen (N), sulfur (S), phosphorus (P), and silicon (Si).

As used herein, a prodrug is a compound that upon in vivo administration is metabolized, or otherwise undergoes chemical changes under physiological conditions, by one or more steps or processes or otherwise converted to a biologically, pharmaceutically or therapeutically active form of the compound. Additionally, prodrugs can be converted to a biologically, pharmaceutically or therapeutically active form of the compound by chemical or biochemical methods in an ex vivo environment. For example, prodrugs can be converted to the compounds of the present invention when placed in a transdermal patch reservoir with a suitable enzyme or chemical reagent.

Certain compounds provided herein can exist in unsolvated forms as well as solvated forms, including hydrated forms. In general, the solvated forms are equivalent to unsolvated forms and are encompassed within the scope of the present disclosure. Certain compounds provided herein may exist in multiple crystalline or amorphous forms. In general, all physical forms are equivalent for the uses contemplated herein and are intended to be within the scope of the present disclosure.

Certain compounds provided herein possess asymmetric carbon atoms (optical centers) or double bonds; the racemates, diastereomers, tautomers, geometric isomers and individual isomers are encompassed within the scope of the present disclosure. The compounds provided herein do not include those which are known in the art to be too unstable to synthesize and/or isolate.

The compounds provided herein may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds provided herein, whether radioactive or not, are encompassed within the scope of the present disclosure.

### II. COMPOUNDS FOR USE IN COMPOSITIONS AND METHODS

In one embodiment, provided herein is a compound for use in the compositions and methods provided herein having Formula I:

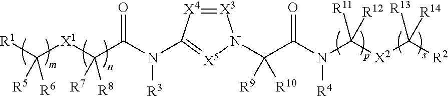

or a pharmaceutically acceptable derivative thereof, wherein:
m, n, p and s are each independently an integer from 0-4;
R¹ and R² are each independently aryl or heteroaryl;
X¹ and X² are each independently a bond, O or NR¹⁵;
X³ and X⁴ are each independently CR¹⁶ or N;
X⁵ is CR¹⁷ or N; and
R³ to R¹⁷ are selected from (i) to (ix):
(i) R³, R⁴ and R¹⁵ are each independently H, alkyl, aryl or aralkyl, and R⁵ to R¹⁴, R¹⁶ and R¹⁷ are each independently H, halo, alkyl, aryl or aralkyl;
(ii) R³ and R⁷, together with the atoms to which they are attached, form a 5-8 membered heterocyclic ring, R⁴ and R¹⁵ are each independently H, alkyl, aryl or aralkyl, and R⁵, R⁶, R⁸ to R¹⁴, R¹⁶ and R¹⁷ are each independently H, halo, alkyl, aryl or aralkyl;
(iii) R⁴ and R⁹, together with the atoms to which they are attached, form a 5-8 membered heterocyclic ring, R³ and R¹⁵ are each independently H, alkyl, aryl or aralkyl, and R⁵ to R⁸, R¹⁰ to R¹⁴, R¹⁶ and R¹⁷ are each independently H, halo, alkyl, aryl or aralkyl;
(iv) R³ and R⁷, together with the atoms to which they are attached, form a 5-8 membered heterocyclic ring, R⁴ and R⁹, together with the atoms to which they are attached, form a 5-8 membered heterocyclic ring, R¹⁵ is independently H, alkyl, aryl or aralkyl, and R⁵, R⁶, R⁸, R¹⁰ to R¹⁴, R¹⁶ and R¹⁷ are each independently H, halo, alkyl, aryl or aralkyl;
(v) R⁴ and R¹¹, together with the atoms to which they are attached, form a 3-8 membered heterocyclic ring, R³ and R¹⁵ are each independently H, alkyl, aryl or aralkyl, and R⁵ to R¹⁰, R¹² to R¹⁴, R¹⁶ and R¹⁷ are each independently H, halo, alkyl, aryl or aralkyl;
(vi) R¹¹ and R¹², together with the atoms to which they are attached, form a 3-8 membered heterocyclic ring, R³, R⁴ and R¹⁵ are each independently H, alkyl, aryl or aralkyl, and R⁵ to R¹⁰, R¹³, R¹⁴, R¹⁶ and R¹⁷ are each independently H, halo, alkyl, aryl or aralkyl;
(vii) when X² is NR¹⁵, R¹¹ and R¹⁵, together with the atoms to which they are attached, form a 3-8 membered heterocyclic ring, R³ and R⁴ are each independently H, alkyl, aryl or aralkyl, and R⁵ to R¹⁰, R¹² to R¹⁴, R¹⁶ and R¹⁷ are each independently H, halo, alkyl, aryl or aralkyl;
(viii) R⁷ and R⁸, together with the atoms to which they are attached, form a 3-8 membered heterocyclic ring, R³, R⁴ and R¹⁵ are each independently H, alkyl, aryl or aralkyl, and R⁵, R⁶, R⁹ to R¹⁴, R¹⁶ and R¹⁷ are each independently H, halo, alkyl, aryl or aralkyl; or
(ix) when X¹ is NR¹⁵, R⁷ and R¹⁵, together with the atoms to which they are attached, form a 3-8 membered heterocyclic ring, R³ and R⁴ are each independently H, alkyl, aryl or aralkyl, and R⁵, R⁶, R⁸ to R¹⁴, R¹⁶ and R¹⁷ are each independently H, halo, alkyl, aryl or aralkyl.

In certain embodiments, the compounds for use in the compositions and methods provided herein are selected with the proviso that when X³ is N, X⁴ is CR¹⁶ and X⁵ is CR¹⁷; then R¹⁶ and R¹⁷ are not both methyl or trifluoromethyl. In another embodiment, the compounds for use in the compositions and methods provided herein are selected with the proviso that when X³ is N, X⁴ is CR¹⁶ and X⁵ is CR¹⁷; then R¹⁶ and R¹⁷ are not both alkyl.

In certain embodiments, the compounds for use in the compositions and methods provided herein are selected with the proviso that R⁹ and R¹⁰ are not both alkyl. In another embodiment, the compounds for use in the compositions and methods provided herein are selected with the proviso that R⁹ and R¹⁰ are not both methyl.

In certain embodiments, the compounds for use in the compositions and methods provided herein are selected with the proviso that the compound is not N-(1-(2-(methyl(2-(p -tolyloxy)ethyl)amino)-2-oxoethyl)-1H-pyrazol-4-yl)-3-phenoxypropanamide.

In another embodiment, provided herein is a compound for use in the compositions and methods provided herein having Formula I:

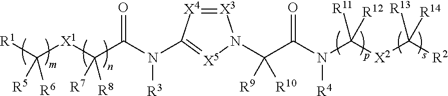

or a pharmaceutically acceptable derivative thereof, wherein:
m, n, p and s are each independently an integer from 0-4;
R¹ and R² are each independently aryl or heteroaryl;
X¹ and X² are each independently a bond, O or NR¹⁵;
X³ and X⁴ are each independently CR¹⁶ or N;
X⁵ is CR¹⁷ or N;
R³, R⁴ and R¹⁵ are each independently H, alkyl, aryl or aralkyl; and
R⁵ to R¹⁴, R¹⁶ and R¹⁷ are each independently H, halo, alkyl, aryl or aralkyl.

In another embodiment, the compounds for use in the compositions and methods provided herein have Formula I, or a pharmaceutically acceptable derivative thereof, wherein:
R¹ and R² are each independently aryl or heteroaryl;
X¹ and X² are each independently a bond or O;
X³ and X⁴ are each independently CH or N;
X⁵ is CH; and
R³ to R¹⁵ are each independently H or alkyl.

In another embodiment, the compounds for use in the compositions and methods provided herein have Formula I, or a pharmaceutically acceptable derivative thereof, wherein:
R¹ and R² are each independently aryl or heteroaryl;
X¹ and X² are each independently a bond or O;
X³ and X⁴ are each independently CH or N;
X⁵ is CH; and
R³ to R¹⁵ are each independently H or methyl.

In another embodiment, the compounds for use in the compositions and methods provided herein have Formula I, or a pharmaceutically acceptable derivative thereof, wherein:
R¹ is aryl;
R² is aryl or heteroaryl;
X¹ is a bond or O;
X² is a bond, O or NR¹⁵;
X³ is CH or N;
X⁴ is CR¹⁶;
X⁵ is CR¹⁷ or N;
R³ to R¹⁷ are each independently H or methyl;
m and s are 0;
n is 0, 1, 2 or 3; and
p is 0 1, 2 or 3.

In another embodiment, the compounds for use in the compositions and methods provided herein have Formula I, or a pharmaceutically acceptable derivative thereof, wherein:
R¹ is aryl;
R² is aryl or heteroaryl;
X¹ is a bond or O;
X² is a bond, O or NCH₃;
X³ is CH or N;
X⁴ is CH or CCH₃;
X⁵ is CH, CCH₃ or N;
R⁴ and R⁹ are each independently H or methyl;
R³, R⁵ to R⁸, and R¹⁰ to R¹⁶ are each H;
m ands are 0;
n is 0, 1, 2 or 3; and
p is 0 1, 2 or 3.

In another embodiment, R¹ is aryl or heteroaryl. In another embodiment, R¹ is phenyl or pyridyl. In another embodiment, R¹ is phenyl or pyridyl, each optionally substituted with halo or haloalkyl. In another embodiment, R¹ is phenyl or pyridyl, each optionally substituted with fluoro or trifluoromethyl. In another embodiment, R¹ is phenyl optionally substituted with halo, alkyl, amino, hydroxy, hydroxyalkyl or alkoxy. In another embodiment, R¹ is phenyl optionally substituted with fluoro, chloro, hydroxymethyl, amino, hydroxy or methoxy. In another embodiment, R¹ is 4-fluorophenyl, 4-trifluoromethylphenyl or 2-pyridyl. In another embodiment, R¹ is phenyl, 2-fluorophenyl, 3-fluorophenyl, 4-fluorophenyl, 4-hydroxymethylphenyl, 4-aminophenyl, 4-chlorophenyl, 4-hydroxyphenyl or 4-methoxyphenyl.

In another embodiment, R² is aryl or heteroaryl. In another embodiment, R² is phenyl or pyridyl. In another embodiment, R² is phenyl or pyridyl, each optionally substituted with alkyl, alkoxy, halo or haloalkyl. In another embodiment, R² is phenyl or pyridyl, each optionally substituted with methyl, methoxy, fluoro or trifluoromethyl. In another embodiment, R² is phenyl or pyridyl, each optionally substituted with alkyl, alkoxy, diazirinyl or haloalkyl. In another embodiment, R² is phenyl or pyridyl, each optionally substituted with methyl, methoxy, 3-(trifluoromethyl)-3H-diazirin-3-yl or trifluoromethyl. In another embodiment, R² is 4-methylphenyl, 4-methoxyphenyl, 4-fluorophenyl, 4-trifluoromethylphenyl or 2-pyridyl. In another embodiment, R² is phenyl, 4-methylphenyl, 4-trifluoromethylphenyl, 2-pyridyl, 5-trifluoromethyl-2-pyridyl, 4-methoxyphenyl or 3-(trifluoromethyl)-3H-diazirin-3-yl.

In another embodiment, X¹ is a bond or O. In another embodiment, X¹ is a bond. In another embodiment, X¹ is O.

In another embodiment, X² is a bond, O or NCH₃. In another embodiment, X² is a bond or O. In another embodiment, X² is a bond. In another embodiment, X² is O. In another embodiment, X² is NCH₃.

In another embodiment, X³ is N or CH. In another embodiment, X⁴ is CH or CCH₃. In another embodiment, X³ is CH and X⁴ is N. In another embodiment, X³ is N and X⁴ is CH.

In another embodiment, X⁵ is CR¹⁷. In another embodiment, X⁵ is CH. In another embodiment, X⁵ is CCH₃. In another embodiment, X⁵ is N.

In another embodiment, X³ is CH, X⁴ is N and X⁵ is CH. In another embodiment, X³ is N, X⁴ is CH and X⁵ is CH. In another embodiment, X³ is N, X⁴ is CCH₃ and X⁵ is CCH₃. In another embodiment, X³ is CH, X⁴ is CH and X⁵ is N.

In another embodiment, R³, R⁴ and R¹⁵ are each independently H, alkyl, aryl or aralkyl, and R⁵ to R¹⁴, R¹⁶ and R¹⁷ are each independently H, halo, alkyl, aryl or aralkyl. In another embodiment, R³, R⁴ and R¹⁵ are each independently H or alkyl. In another embodiment, R³, R⁴ and R¹⁵ are each independently H or methyl. In another embodiment, R⁵ to R¹⁴, R¹⁶ and R¹⁷ are each independently H or alkyl. In another embodiment, R⁵ to R¹⁴, R¹⁶ and R¹⁷ are each independently H or methyl. In another embodiment, R⁵ to R¹⁴, R¹⁶ and R¹⁷ are each independently H. In another embodiment, R³, R⁴, R⁹, R¹⁶ and R¹⁷ are H or methyl, R⁵ to R⁸ and R¹⁰ to R¹⁴ are H, and R¹⁵ is methyl.

In another embodiment, R³ is H or alkyl. In another embodiment, R³ is H or methyl. In another embodiment, R³ is H. In another embodiment, R³ is methyl.

In another embodiment, R⁴ is H or alkyl. In another embodiment, R⁴ is H or methyl. In another embodiment, R⁴ is H. In another embodiment, R⁴ is methyl.

In another embodiment, R⁷ is H or alkyl. In another embodiment, R⁷ is H or methyl. In another embodiment, R⁷ is H. In another embodiment, R⁷ is methyl.

In another embodiment, R⁹ is H or alkyl. In another embodiment, R⁹ is H or methyl. In another embodiment, R⁹ is H. In another embodiment, R⁹ is methyl.

In another embodiment, m is 0. In another embodiment, n is 0, 1, 2 or 3. In another embodiment, n is 2. In another embodiment, p is 0, 1, 2 or 3. In another embodiment, p is 2. In another embodiment, s is 0.

In another embodiment, the compounds provided herein for use in the compositions and methods provided herein are those shown in Table 1 below:

| Compound | Structure |
|---|---|
| 1 | 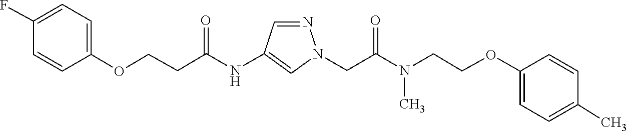 [SMILES: Cc1ccc(OCCN(C)C(=O)Cn2cc(NC(=O)CCOc3ccc(F)cc3)cn2)cc1] |
| 2 | 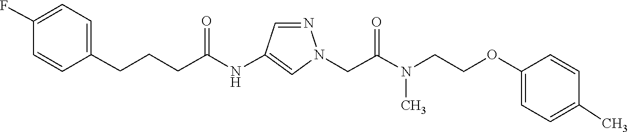 [SMILES: Cc1ccc(OCCN(C)C(=O)Cn2cc(NC(=O)CCCc3ccc(F)cc3)cn2)cc1] |
| 3 | 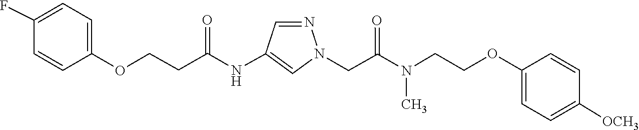 [SMILES: COc1ccc(OCCN(C)C(=O)Cn2cc(NC(=O)CCOc3ccc(F)cc3)cn2)cc1] |
| 4 | 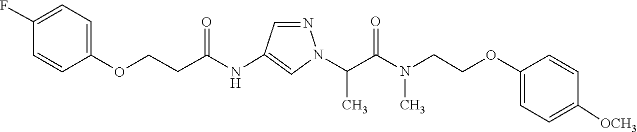 [SMILES: COc1ccc(OCCN(C)C(=O)C(C)n2cc(NC(=O)CCOc3ccc(F)cc3)cn2)cc1] |
| 5 | 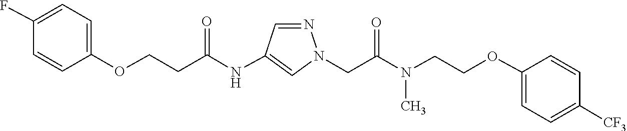 [SMILES: CN(CCOc1ccc(C(F)(F)F)cc1)C(=O)Cn1cc(NC(=O)CCOc2ccc(F)cc2)cn1] |
| 6 | 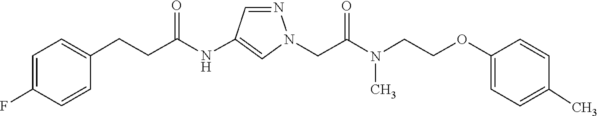 [SMILES: Cc1ccc(OCCN(C)C(=O)Cn2cc(NC(=O)CCc3ccc(F)cc3)cn2)cc1] |
| 7 | 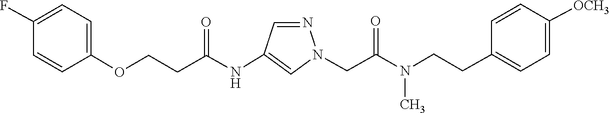 [SMILES: COc1ccc(CCN(C)C(=O)Cn2cc(NC(=O)CCOc3ccc(F)cc3)cn2)cc1] |
| 8 | 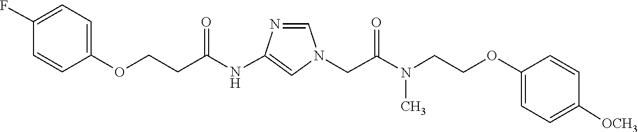 [SMILES: COc1ccc(OCCN(C)C(=O)Cn2cnc(NC(=O)CCOc3ccc(F)cc3)c2)cc1] |
| 9 | 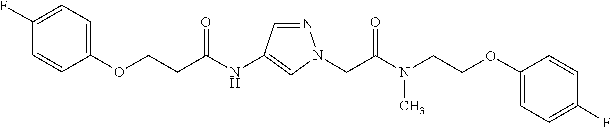 [SMILES: CN(CCOc1ccc(F)cc1)C(=O)Cn1cc(NC(=O)CCOc2ccc(F)cc2)cn1] |
| 10 | 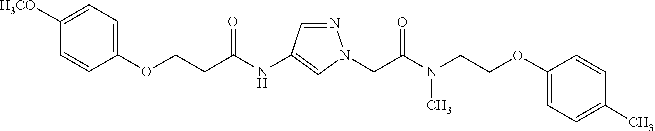 [SMILES: COc1ccc(OCCC(=O)Nc2cnn(CC(=O)N(C)CCOc3ccc(C)cc3)c2)cc1] |
| 11 | 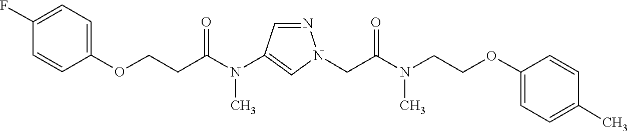 [SMILES: Cc1ccc(OCCN(C)C(=O)Cn2cc(N(C)C(=O)CCOc3ccc(F)cc3)cn2)cc1] |
| 12 | 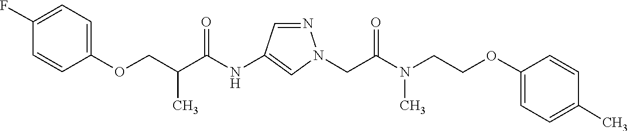 [SMILES: Cc1ccc(OCCN(C)C(=O)Cn2cc(NC(=O)C(C)COc3ccc(F)cc3)cn2)cc1] |
| 13 | 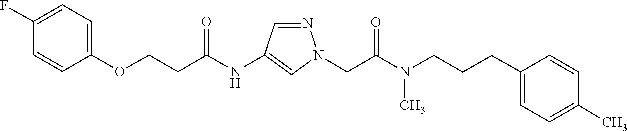 [SMILES: Cc1ccc(CCCN(C)C(=O)Cn2cc(NC(=O)CCOc3ccc(F)cc3)cn2)cc1] |
| 14 | 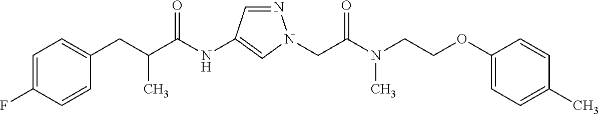 |
| 15 | 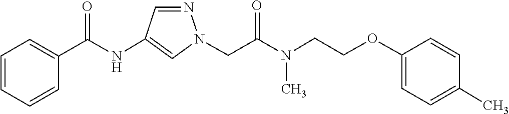 [SMILES: Cc1ccc(OCCN(C)C(=O)Cn2cc(NC(=O)c3ccccc3)cn2)cc1] |
| 16 | 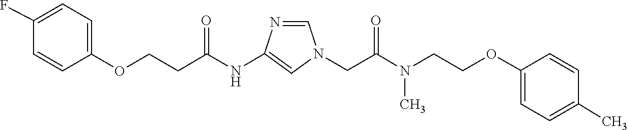 [SMILES: Cc1ccc(OCCN(C)C(=O)Cn2cnc(NC(=O)CCOc3ccc(F)cc3)c2)cc1] |
| 17 | 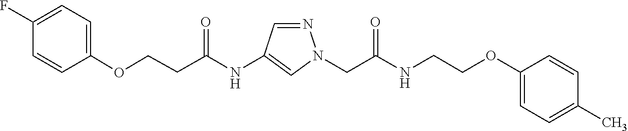 [SMILES: Cc1ccc(OCCNC(=O)Cn2cc(NC(=O)CCOc3ccc(F)cc3)cn2)cc1] |
| 18 | 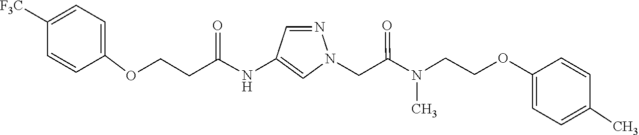 [SMILES: Cc1ccc(OCCN(C)C(=O)Cn2cc(NC(=O)CCOc3ccc(C(F)(F)F)cc3)cn2)cc1] |
| 19 | 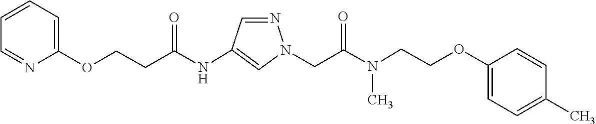 [SMILES: Cc1ccc(OCCN(C)C(=O)Cn2cc(NC(=O)CCOc3ccccn3)cn2)cc1] |
| 20 | 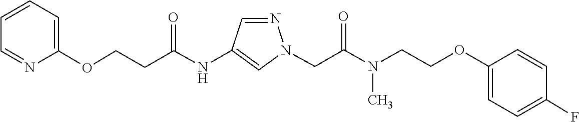 [SMILES: CN(CCOc1ccc(F)cc1)C(=O)Cn1cc(NC(=O)CCOc2ccccn2)cn1] |
| 21 | 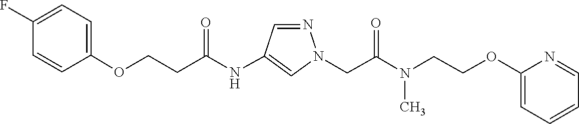 [SMILES: CN(CCOc1ccccn1)C(=O)Cn1cc(NC(=O)CCOc2ccc(F)cc2)cn1] |
| 22 | 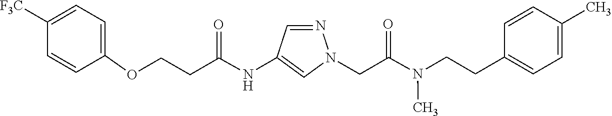 |
| 23 | 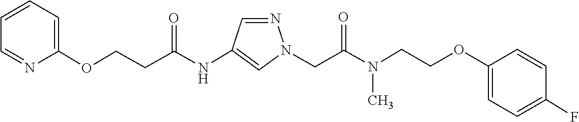 [SMILES: CN(CCOc1ccc(F)cc1)C(=O)Cn1cc(NC(=O)CCOc2ccccn2)cn1] |
| 24 | 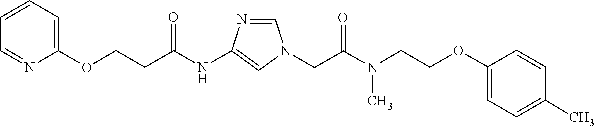 [SMILES: Cc1ccc(OCCN(C)C(=O)Cn2cnc(NC(=O)CCOc3ccccn3)c2)cc1] |
| 25 | 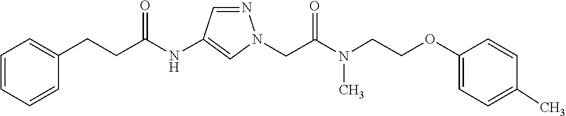 [SMILES: Cc1ccc(OCCN(C)C(=O)Cn2cc(NC(=O)CCc3ccccc3)cn2)cc1] |
| 26 | 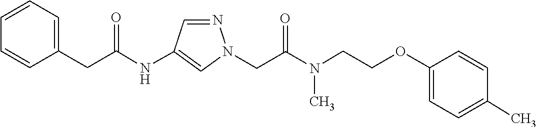 [SMILES: Cc1ccc(OCCN(C)C(=O)Cn2cc(NC(=O)Cc3ccccc3)cn2)cc1] |
| 27 | 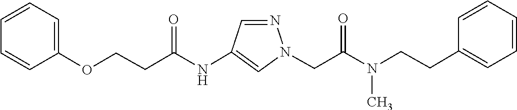 [SMILES: CN(CCc1ccccc1)C(=O)Cn1cc(NC(=O)CCOc2ccccc2)cn1] |
| 28 | 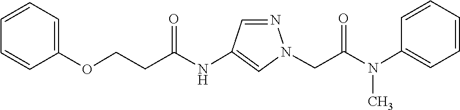 [SMILES: CN(C(=O)Cn1cc(NC(=O)CCOc2ccccc2)cn1)c1ccccc1] |
| 29 | 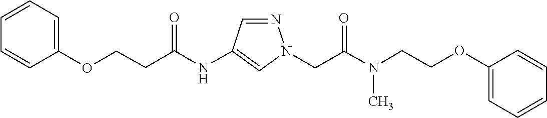 [SMILES: CN(CCOc1ccccc1)C(=O)Cn1cc(NC(=O)CCOc2ccccc2)cn1] |
| 30 | 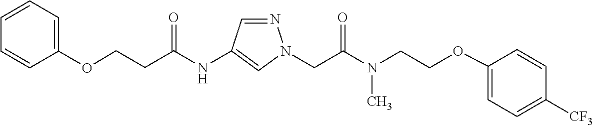 [SMILES: CN(CCOc1ccc(C(F)(F)F)cc1)C(=O)Cn1cc(NC(=O)CCOc2ccccc2)cn1] |
| 31 | 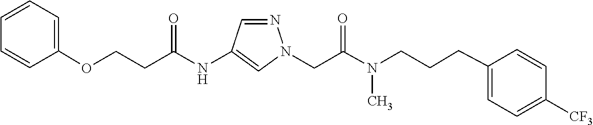 [SMILES: CN(CCCc1ccc(C(F)(F)F)cc1)C(=O)Cn1cc(NC(=O)CCOc2ccccc2)cn1] |
| 32 | 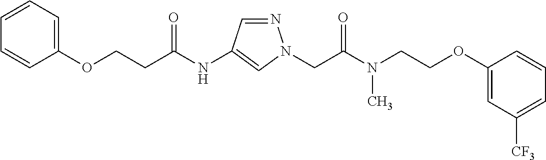 [SMILES: CN(CCOc1cccc(C(F)(F)F)c1)C(=O)Cn1cc(NC(=O)CCOc2ccccc2)cn1] |
| 33 | 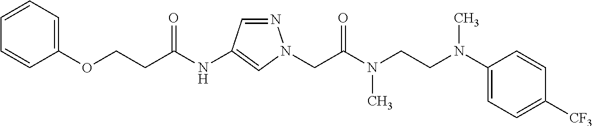 [SMILES: CN(CCN(C)c1ccc(C(F)(F)F)cc1)C(=O)Cn1cc(NC(=O)CCOc2ccccc2)cn1] |
| 34 | 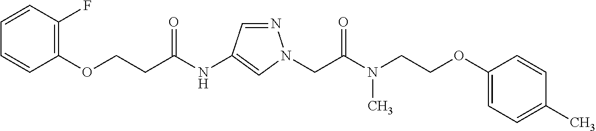 [SMILES: Cc1ccc(OCCN(C)C(=O)Cn2cc(NC(=O)CCOc3ccccc3F)cn2)cc1] |
| 35 | 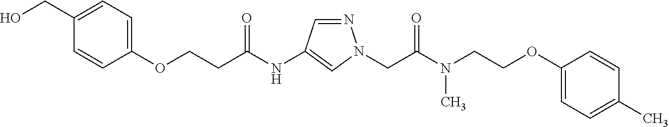 [SMILES: Cc1ccc(OCCN(C)C(=O)Cn2cc(NC(=O)CCOc3ccc(CO)cc3)cn2)cc1] |
| 36 | 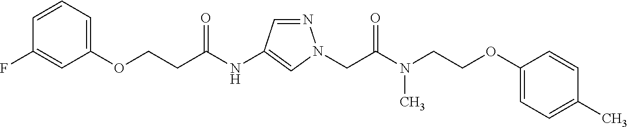 [SMILES: Cc1ccc(OCCN(C)C(=O)Cn2cc(NC(=O)CCOc3cccc(F)c3)cn2)cc1] |
| 37 | 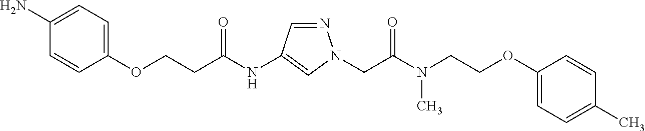 [SMILES: Cc1ccc(OCCN(C)C(=O)Cn2cc(NC(=O)CCOc3ccc(N)cc3)cn2)cc1] |
| 38 | 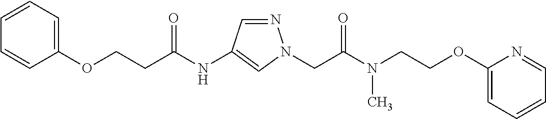 [SMILES: CN(CCOc1ccccn1)C(=O)Cn1cc(NC(=O)CCOc2ccccc2)cn1] |
| 39 | 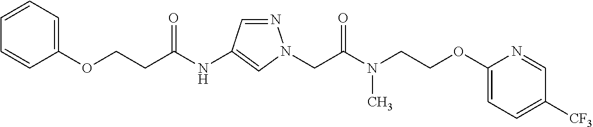 [SMILES: CN(CCOc1ccc(C(F)(F)F)cn1)C(=O)Cn1cc(NC(=O)CCOc2ccccc2)cn1] |
| 40 | 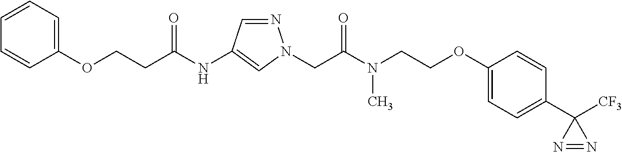 [SMILES: CN(CCOc1ccc(C2(C(F)(F)F)N=N2)cc1)C(=O)Cn1cc(NC(=O)CCOc2ccccc2)cn1] |
| 41 | 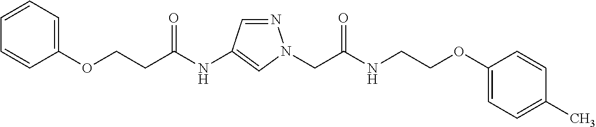 [SMILES: Cc1ccc(OCCNC(=O)Cn2cc(NC(=O)CCOc3ccccc3)cn2)cc1] |
| 42 | 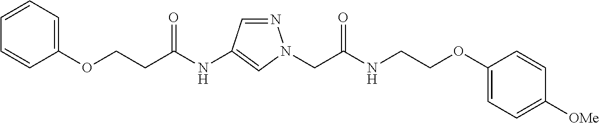 [SMILES: COc1ccc(OCCNC(=O)Cn2cc(NC(=O)CCOc3ccccc3)cn2)cc1] |
| 43 | 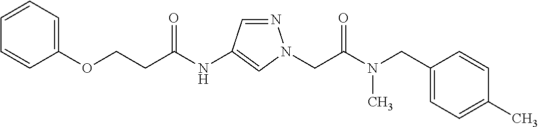 [SMILES: Cc1ccc(CN(C)C(=O)Cn2cc(NC(=O)CCOc3ccccc3)cn2)cc1] |
| 44 | 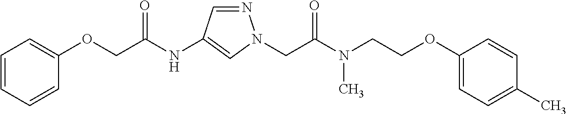 [SMILES: Cc1ccc(OCCN(C)C(=O)Cn2cc(NC(=O)COc3ccccc3)cn2)cc1] |
| 45 | 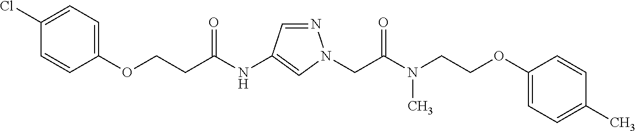 [SMILES: Cc1ccc(OCCN(C)C(=O)Cn2cc(NC(=O)CCOc3ccc(Cl)cc3)cn2)cc1] |
| 46 | 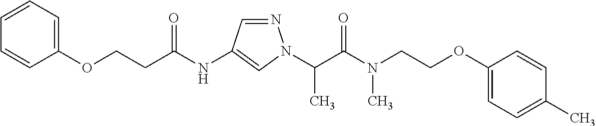 [SMILES: Cc1ccc(OCCN(C)C(=O)C(C)n2cc(NC(=O)CCOc3ccccc3)cn2)cc1] |
| 47 | 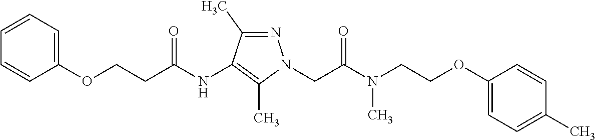 [SMILES: Cc1ccc(OCCN(C)C(=O)Cn2nc(C)c(NC(=O)CCOc3ccccc3)c2C)cc1] |
| 48 | 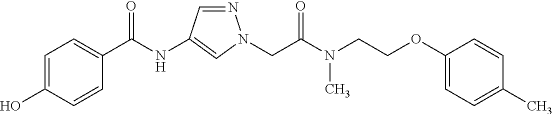 [SMILES: Cc1ccc(OCCN(C)C(=O)Cn2cc(NC(=O)c3ccc(O)cc3)cn2)cc1] |
| 49 | 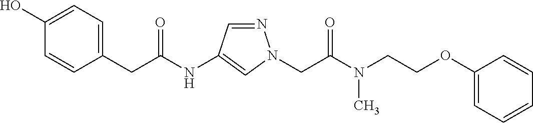 [SMILES: CN(CCOc1ccccc1)C(=O)Cn1cc(NC(=O)Cc2ccc(O)cc2)cn1] |
| 50 | 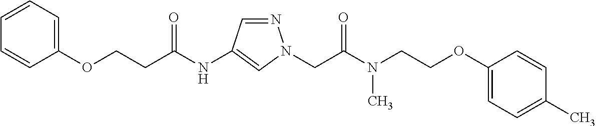 [SMILES: Cc1ccc(OCCN(C)C(=O)Cn2cc(NC(=O)CCOc3ccccc3)cn2)cc1] |
| 51 | 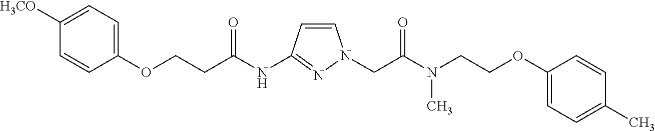 [SMILES: COc1ccc(OCCC(=O)Nc2ccn(CC(=O)N(C)CCOc3ccc(C)cc3)n2)cc1] |
| 52 | 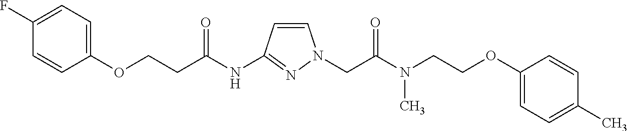 [SMILES: Cc1ccc(OCCN(C)C(=O)Cn2ccc(NC(=O)CCOc3ccc(F)cc3)n2)cc1] |
| 53 | 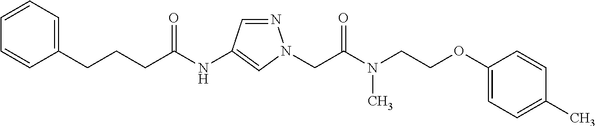 [SMILES: Cc1ccc(OCCN(C)C(=O)Cn2cc(NC(=O)CCCc3ccccc3)cn2)cc1] |

### III. SYNTHESIS OF THE COMPOUNDS

The compounds provided herein may be prepared according to synthetic methods well known to those of skill in the art. In one embodiment, the compounds may be prepared according to Scheme I:

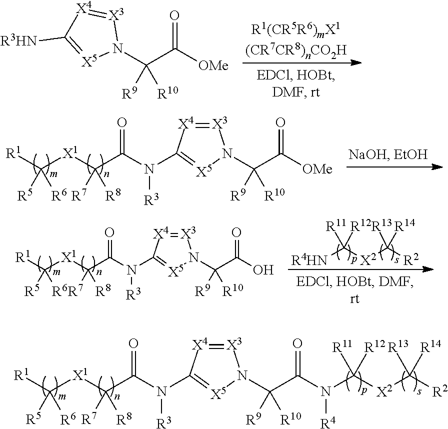

In another embodiment, the compounds may be prepared according to Scheme II:

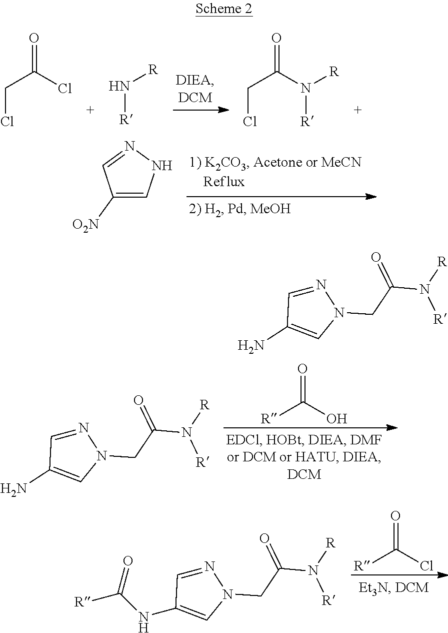

In another embodiment, the compounds may be prepared according to Scheme III:

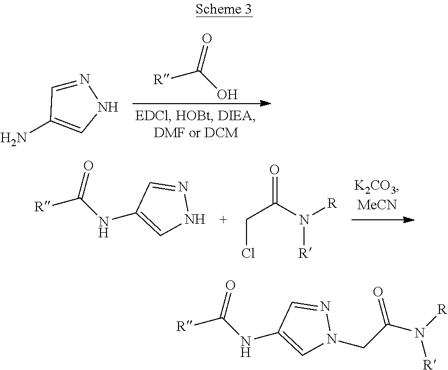

In another embodiment, the compounds may be prepared according to Scheme IV:

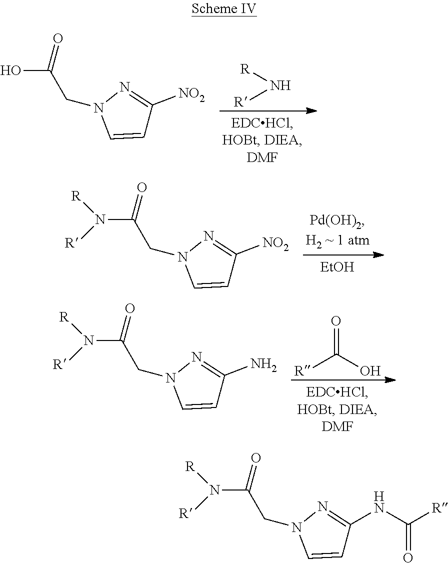

### IV. PHARMACEUTICAL COMPOSITIONS

The pharmaceutical compositions provided herein contain therapeutically effective amounts of one or more of compounds provided herein and a pharmaceutically acceptable carrier, diluent or excipient.

The compounds can be formulated into suitable pharmaceutical preparations such as solutions, suspensions, tablets, dispersible tablets, pills, capsules, powders, sustained release formulations or elixirs, for oral administration or in sterile solutions or suspensions for ophthalmic or parenteral administration, as well as transdermal patch preparation and dry powder inhalers. Typically, the compounds described above are formulated into pharmaceutical compositions using techniques and procedures well known in the art (see, e.g., Ansel Introduction to Pharmaceutical Dosage Forms, Seventh Edition 1999).

In the compositions, effective concentrations of one or more compounds or pharmaceutically acceptable salts is (are) mixed with a suitable pharmaceutical carrier or vehicle. In certain embodiments, the concentrations of the compounds in the compositions are effective for delivery of an amount, upon administration, that treats, prevents, or ameliorates one or more of the symptoms and/or progression of a disease or disorder disclosed herein.

Typically, the compositions are formulated for single dosage administration. To formulate a composition, the weight fraction of compound is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients. Liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as known in the art. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The active compound is included in the pharmaceutically acceptable carrier in an amount sufficient to exert a therapeutically useful effect in the absence of undesirable side effects on the subject treated. The therapeutically effective concentration may be determined empirically by testing the compounds in in vitro and in vivo systems described herein and then extrapolated therefrom for dosages for humans. In some embodiments, the active compound is administered in a method to achieve a therapeutically effective concentration of the drug. In some embodiments, a companion diagnostic (see, e.g., Olsen D and Jorgensen J T, Front. Oncol., 2014 May 16, 4:105, doi: 10.3389/fonC.2014.00105) is used to determine the therapeutic concentration and safety profile of the active compound in specific subjects or subject populations.

The concentration of active compound in the pharmaceutical composition will depend on absorption, tissue distribution, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of a disease or disorder disclosed herein.

In certain embodiments, a therapeutically effective dosage should produce a serum concentration of active ingredient of from about 0.1 ng/mL to about 50-100 μg/mL. In one embodiment, the pharmaceutical compositions provide a dosage of from about 0.001 mg to about 2000 mg of compound per kilogram of body weight per day. Pharmaceutical dosage unit forms are prepared to provide from about 1 mg to about 1000 mg and in certain embodiments, from about 10 to about 500 mg of the essential active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Thus, effective concentrations or amounts of one or more of the compounds described herein or pharmaceutically acceptable salts thereof are mixed with a suitable pharmaceutical carrier or vehicle for systemic, topical or local administration to form pharmaceutical compositions. Compounds are included in an amount effective for ameliorating one or more symptoms of, or for treating, retarding progression, or preventing. The concentration of active compound in the composition will depend on absorption, tissue distribution, inactivation, excretion rates of the active compound, the dosage schedule, amount administered, particular formulation as well as other factors known to those of skill in the art.

The compositions are intended to be administered by a suitable route, including but not limited to oral, parenteral, subcutaneous, intravenous, intramuscular, intraperitoneal, intrathecal, mucosal, dermal, transdermal, buccal, rectal, topical, local, nasal or inhalation. For oral administration, capsules and tablets can be formulated. The compositions are in liquid, semi-liquid or solid form and are formulated in a manner suitable for each route of administration.

Solutions or suspensions used for parenteral, intradermal, subcutaneous, or topical application can include any of the following components: a sterile diluent, such as water for injection, saline solution, fixed oil, polyethylene glycol, glycerin, propylene glycol, dimethyl acetamide or other synthetic solvent; antimicrobial agents, such as benzyl alcohol and methyl parabens; antioxidants, such as ascorbic acid and sodium bisulfate; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, pens, disposable syringes or single or multiple dose vials made of glass, plastic or other suitable material.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil water emulsions containing suitable quantities of the compounds or pharmaceutically acceptable salts thereof. The pharmaceutically therapeutically active compounds and salts thereof are formulated and administered in unit dosage forms or multiple dosage forms. Unit dose forms as used herein refer to physically discrete units suitable for human and animal subjects and packaged individually as is known in the art. Each unit dose contains a predetermined quantity of the therapeutically active compound sufficient to produce the desired therapeutic effect, in association with the required pharmaceutical carrier, vehicle or diluent. Examples of unit dose forms include ampules and syringes and individually packaged tablets or capsules. Unit dose forms may be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses which are not segregated in packaging.

Sustained-release preparations can also be prepared. Suitable examples of sustained-release preparations include semipermeable matrices of solid hydrophobic polymers containing the compound provided herein, which matrices are in the form of shaped articles, e.g., films, or microcapsule. Examples of sustained-release matrices include iontophoresis patches, polyesters, hydrogels (for example, poly(2-hydroxyethyl-methacrylate), or poly(vinylalcohol)), polylactides, copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(-)-3-hydroxybutyric acid. While polymers such as ethylene-vinyl acetate and lactic acid-glycolic acid enable release of molecules for over 100 days, certain hydrogels release proteins for shorter time periods. When encapsulated compound remain in the body for a long time, they may denature or aggregate as a result of exposure to moisture at 37° C., resulting in a loss of biological activity and possible changes in their structure. Rational strategies can be devised for stabilization depending on the mechanism of action involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Dosage forms or compositions containing active ingredient in the range of 0.005% to 100% with the balance made up from non-toxic carrier may be prepared. For oral administration, a pharmaceutically acceptable non-toxic composition is formed by the incorporation of any of the normally employed excipients, such as, for example pharmaceutical grades of mannitol, lactose, starch, magnesium stearate, talcum, cellulose derivatives, sodium croscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions may contain about 0.001% 100% active ingredient, in certain embodiments, about 0.1 85% active ingredient, or, in other embodiments, about 75-95% active ingredient.

The active compounds or pharmaceutically acceptable salts may be prepared with carriers that protect the compound against rapid elimination from the body, such as time release formulations or coatings.

The compositions may include other active compounds to obtain desired combinations of properties. The compounds provided herein, or pharmaceutically acceptable salts thereof as described herein, may also be advantageously administered for therapeutic or prophylactic purposes together with another pharmacological agent known in the general art to be of value in treating one or more of the diseases or medical conditions referred to hereinabove, such as diseases related to oxidative stress. It is to be understood that such combination therapy constitutes a further aspect of the compositions and methods of treatment provided herein.

Lactose-free compositions provided herein can contain excipients that are well known in the art and are listed, for example, in the U.S. Pharmacopeia (USP) SP (XXI)/NF (XVI). In general, lactose-free compositions contain an active ingredient, a binder/filler, and a lubricant in pharmaceutically compatible and pharmaceutically acceptable amounts. Exemplary lactose-free dosage forms contain an active ingredient, microcrystalline cellulose, pre-gelatinized starch and magnesium stearate.

Further encompassed are anhydrous pharmaceutical compositions and dosage forms containing a compound provided herein. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment and use of formulations.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine are anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs and strip packs.

### A. Oral Dosage Forms

Oral pharmaceutical dosage forms are either solid, gel or liquid. The solid dosage forms are tablets, capsules, granules, and bulk powders. Types of oral tablets include compressed, chewable lozenges and tablets which may be enteric coated, sugar coated or film coated. Capsules may be hard or soft gelatin capsules, while granules and powders may be provided in non-effervescent or effervescent form with the combination of other ingredients known to those skilled in the art.

In certain embodiments, the formulations are solid dosage forms, such as capsules or tablets. The tablets, pills, capsules, troches and the like can contain any of the following ingredients, or compounds of a similar nature: a binder; a diluent; a disintegrating agent; a lubricant; a glidant; a sweetening agent; and a flavoring agent.

Examples of binders include microcrystalline cellulose, gum tragacanth, glucose solution, acacia mucilage, gelatin solution, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include croscarmellose sodium, sodium starch glycolate, crospovidone, alginic acid, corn starch, potato starch, bentonite, methylcellulose, agar and carboxymethylcellulose. Coloring agents include, for example, any of the approved certified water-soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene lauryl ether. Emetic coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

If oral administration is desired, the compound could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable salt thereof as described herein. Higher concentrations, up to about 98% by weight of the active ingredient may be included.

Pharmaceutically acceptable carriers included in tablets are binders, lubricants, diluents, disintegrating agents, coloring agents, flavoring agents, and wetting agents. Enteric coated tablets, because of the enteric coating, resist the action of stomach acid and dissolve or disintegrate in the neutral or alkaline intestines. Sugar coated tablets are compressed tablets to which different layers of pharmaceutically acceptable substances are applied. Film coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar coated, multiple compressed and chewable tablets. Flavoring and sweetening agents are especially useful in the formation of chewable tablets and lozenges.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil in-water or water in oil. In some embodiments, the suspension is a suspension of microparticles or nanoparticles. In some embodiments, the emulsion is an emulsion of microparticles or nanoparticles.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic adds include citric and tartaric acid. Sources of carbon dioxide include sodium bicarbonate and sodium carbonate. Coloring agents include any of the approved certified water-soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include, but are not limited to, those containing a compound provided herein, a dialkylated mono- or poly-alkylene glycol, including, but not limited to, 1,2-dimethoxyethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

### B. Injectables, Solutions and Emulsions

Parenteral administration, generally characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. In some embodiments, the suspension is a suspension of microparticles or nanoparticles. In some embodiments, the emulsion is an emulsion of microparticles or nanoparticles. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow release or sustained release system, such that a constant level of dosage is maintained is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple dose containers which include phenols or cresols, mercurials, benzyl alcohol, chlorobutanol, methyl and propyl p hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the subject or animal as is known in the art.

The unit dose parenteral preparations are packaged in an ampule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. Typically, a therapeutically effective dosage is formulated to contain a concentration of at least about 0.1% w/w up to about 90% w/w or more, such as more than 1% w/w of the active compound to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

### C. Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable salt thereof, in a suitable solvent. The solvent may contain an excipient which improves the stability or other pharmacological component of the powder or reconstituted solution, prepared from the powder. Excipients that may be used include, but are not limited to, dextrose, sorbitol, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (including but not limited to 10-1000 mg or 100-500 mg) or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, about 5-35 mg, or about 9-30 mg of lyophilized powder, is added per mL of sterile water or other suitable carrier. The precise amount depends upon the selected compound. Such amount can be empirically determined.

### D. Topical Administration

Topical mixtures are prepared as described for the local and systemic administration. The resulting mixture may be a solution, suspension, emulsion or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable salts thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will have diameters of less than 50 microns or less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% isotonic solutions, pH about 5-7, with appropriate salts.

### E. Compositions for Other Routes of Administration

Other routes of administration, such as topical application, transdermal patches, and rectal administration are also contemplated herein.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin gelatin, carbowax (polyoxyethylene glycol) and appropriate mixtures of mono, di and triglycerides of fatty acids. Combinations of the various bases may be used. Agents to raise the melting point of suppositories include spermaceti and wax. Rectal suppositories may be prepared either by the compressed method or by molding. An exemplary weight of a rectal suppository is about 2 to 3 grams.

Tablets and capsules for rectal administration are manufactured using the same pharmaceutically acceptable substance and by the same methods as for formulations for oral administration.

### F. Sustained Release Compositions

Active ingredients provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, 5,639,480, 5,733,566, 5,739,108, 5,891,474, 5,922,356, 5,972,891, 5,980,945, 5,993,855, 6,045,830, 6,087,324, 6,113,943, 6,197,350, 6,248,363, 6,264,970, 6,267,981, 6,376,461, 6,419,961, 6,589,548, 6,613,358, 6,699,500 and 6,740,634, each of which is incorporated herein by reference. Such dosage forms can be used to provide slow or controlled-release of one or more active ingredients using, for example, hydroxypropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients provided herein.

All controlled-release pharmaceutical products have a common goal of improving drug therapy over that achieved by their non-controlled counterparts. In one embodiment, the use of an optimally designed controlled-release preparation in medical treatment is characterized by a minimum of drug substance being employed to cure or control the condition in a minimum amount of time. In certain embodiments, advantages of controlled-release formulations include extended activity of the drug, reduced dosage frequency, and increased subject compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled release of an active ingredient can be stimulated by various conditions including, but not limited to, pH, temperature, enzymes, water, or other physiological conditions or compounds.

In certain embodiments, the agent may be administered using intravenous infusion, an implantable osmotic pump, a transdermal patch, liposomes, or other modes of administration. In one embodiment, a pump may be used (see, Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity of the therapeutic target, i.e., thus requiring only a fraction of the systemic dose (see, e.g., Goodson, Medical Applications of Controlled Release, vol. 2, pp. 115-138 (1984).

In some embodiments, a controlled release device is introduced into a subject in proximity of the site of inappropriate immune activation or a tumor. Other controlled release systems are discussed in the review by Langer (Science 249:1527-1533 (1990). The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient contained in such parenteral compositions is highly dependent on the specific nature thereof, as well as the needs of the subject.

### G. Targeted Formulations

The compounds provided herein, or pharmaceutically acceptable salts thereof, may also be formulated to be targeted to a particular tissue, receptor, or other area of the body of the subject to be treated, including liposome-, resealed erythrocyte-, and antibody-based delivery systems. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In one embodiment, the antibody-based delivery system is an antibody-drug conjugate (“ADC”), e.g., as described in Hamilton G S, Biologicals, 2015 September, 43(5):318-32; Kim E G and Kim K M, Biomol. Ther. (Seoul), 2015 November, 23(6):493-509; and Peters C and Brown S, Biosci. Rep., 2015 Jun. 12, 35(4) pii: e00225, each of which is incorporated herein by reference.

In one embodiment, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

### H. Articles Of Manufacture

The compounds or pharmaceutically acceptable salts can be packaged as articles of manufacture containing packaging material, a compound or pharmaceutically acceptable salt thereof provided herein, which is used for treatment, prevention or amelioration of one or more symptoms or progression of a disease or disorder disclosed herein, and a label that indicates that the compound or pharmaceutically acceptable salt thereof is used for treatment, prevention or amelioration of one or more symptoms or progression of a disease or disorder disclosed herein.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, pens, bottles, and any packaging material suitable for a selected formulation and intended mode of administration and treatment. A wide array of formulations of the compounds and compositions provided herein are contemplated.

In certain embodiments, provided herein also are kits which, when used by the medical practitioner, can simplify the administration of appropriate amounts of active ingredients to a subject. In certain embodiments, the kit provided herein includes a container and a dosage form of a compound provided herein, including a single enantiomer or a mixture of diastereomers thereof or a pharmaceutically acceptable salt, solvate, or prodrug thereof.

In certain embodiments, the kit includes a container comprising a dosage form of the compound provided herein, including a single enantiomer or a mixture of diastereomers thereof; or a pharmaceutically acceptable salt, solvate, or prodrug thereof, in a container comprising one or more other therapeutic agent(s) described herein.

Kits provided herein can further include devices that are used to administer the active ingredients. Examples of such devices include, but are not limited to, syringes, needle-less injectors drip bags, patches, and inhalers. The kits provided herein can also include condoms for administration of the active ingredients.

Kits provided herein can further include pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: aqueous vehicles, including, but not limited to, Water for Injection USP, Sodium Chloride Injection, Ringer's Injection, Dextrose Injection, Dextrose and Sodium Chloride Injection, and Lactated Ringer's Injection; water-miscible vehicles, including, but not limited to, ethyl alcohol, polyethylene glycol, and polypropylene glycol; and non-aqueous vehicles, including, but not limited to, corn oil, cottonseed oil, peanut oil, sesame oil, ethyl oleate, isopropyl myristate, and benzyl benzoate.

### V. DOSING

The compounds and pharmaceutical compositions provided herein may be dosed in certain therapeutically or prophylactically effective amounts, certain time intervals, certain dosage forms, and certain dosage administration methods as described below.

In certain embodiments, a therapeutically or prophylactically effective amount of the compound is from about 0.005 to about 1,000 mg per day, from about 0.01 to about 500 mg per day, from about 0.01 to about 250 mg per day, from about 0.01 to about 100 mg per day, from about 0.1 to about 100 mg per day, from about 0.5 to about 100 mg per day, from about 1 to about 100 mg per day, from about 0.01 to about 50 mg per day, from about 0.1 to about 50 mg per day, from about 0.5 to about 50 mg per day, from about 1 to about 50 mg per day, from about 0.02 to about 25 mg per day, from about 0.05 to about 10 mg per day, from about 0.05 to about 5 mg per day, from about 0.1 to about 5 mg per day, or from about 0.5 to about 5 mg per day.

In certain embodiments, the therapeutically or prophylactically effective amount is about 0.1, about 0.2, about 0.5, about 1, about 2, about 3, about 4, about 5, about 6, about 7, about 8, about 9, about 10, about 15, about 20, about 25, about 30, about 40, about 45, about 50, about 60, about 70, about 80, about 90, about 100, or about 150 mg per day.

In one embodiment, the recommended daily dose range of the compound provided herein, or a derivative thereof, for the conditions described herein lie within the range of from about 0.5 mg to about 50 mg per day, in one embodiment given as a single once-a-day dose, or in divided doses throughout a day. In some embodiments, the dosage ranges from about 1 mg to about 50 mg per day. In other embodiments, the dosage ranges from about 0.5 to about 5 mg per day. Specific doses per day include 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mg per day.

In a specific embodiment, the recommended starting dosage may be 0.5, 1, 2, 3, 4, 5, 10, 15, 20, 25 or 50 mg per day. In another embodiment, the recommended starting dosage may be 0.5, 1, 2, 3, 4, or 5 mg per day. The dose may be escalated to 15, 20, 25, 30, 35, 40, 45 and 50 mg/day. In a specific embodiment, the compound can be administered in an amount of about 25 mg/day. In a particular embodiment, the compound can be administered in an amount of about 10 mg/day. In a particular embodiment, the compound can be administered in an amount of about 5 mg/day. In a particular embodiment, the compound can be administered in an amount of about 4 mg/day. In a particular embodiment, the compound can be administered in an amount of about 3 mg/day.

In certain embodiments, the therapeutically or prophylactically effective amount is from about 0.001 to about 100 mg/kg/day, from about 0.01 to about 50 mg/kg/day, from about 0.01 to about 25 mg/kg/day, from about 0.01 to about 10 mg/kg/day, from about 0.01 to about 9 mg/kg/day, 0.01 to about 8 mg/kg/day, from about 0.01 to about 7 mg/kg/day, from about 0.01 to about 6 mg/kg/day, from about 0.01 to about 5 mg/kg/day, from about 0.01 to about 4 mg/kg/day, from about 0.01 to about 3 mg/kg/day, from about 0.01 to about 2 mg/kg/day, from about 0.01 to about 1 mg/kg/day, or from about 0.01 to about 0.05 mg/kg/day.

The administered dose can also be expressed in units other than mg/kg/day. For example, doses for parenteral administration can be expressed as mg/m²/day. One of ordinary skill in the art would readily know how to convert doses from mg/kg/day to mg/m²/day to given either the height or weight of a subject or both (see, www.fda.gov/cder/cancer/animalframe.htm). For example, a dose of 1 mg/kg/day for a 65 kg human is approximately equal to 38 mg/m²/day.

In certain embodiments, the amount of the compound administered is sufficient to provide a plasma concentration of the compound at steady state, ranging from about 0.001 to about 500 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM, or from about 1 to about 20 μM.

In other embodiments, the amount of the compound administered is sufficient to provide a plasma concentration of the compound at steady state, ranging from about 5 to about 100 nM, about 5 to about 50 nM, about 10 to about 100 nM, about 10 to about 50 nM or from about 50 to about 100 nM.

As used herein, the term “plasma concentration at steady state” is the concentration reached after a period of administration of a compound provided herein, or a derivative thereof. Once steady state is reached, there are minor peaks and troughs on the time dependent curve of the plasma concentration of the compound.

In certain embodiments, the amount of the compound administered is sufficient to provide a maximum plasma concentration (peak concentration) of the compound, ranging from about 0.001 to about 50 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM, or from about 1 to about 20 μM.

In certain embodiments, the amount of the compound administered is sufficient to provide a minimum plasma concentration (trough concentration) of the compound, ranging from about 0.001 to about 500 μM, about 0.002 to about 200 μM, about 0.005 to about 100 μM, about 0.01 to about 50 μM, from about 1 to about 50 μM, about 0.01 to about 25 μM, from about 0.01 to about 20 μM, from about 0.02 to about 20 μM, from about 0.02 to about 20 μM, or from about 0.01 to about 20 μM.

In certain embodiments, the amount of the compound administered is sufficient to provide an area under the curve (AUC) of the compound, ranging from about 100 to about 100,000 ng*hr/mL, from about 1,000 to about 50,000 ng*hr/mL, from about 5,000 to about 25,000 ng*hr/mL, or from about 5,000 to about 10,000 ng*hr/mL.

The methods provided herein encompass treating a patient regardless of subject's age, although some diseases or disorders are more common in certain age groups.

Depending on the disease to be treated and the subject's condition, the compound provided herein, or a derivative thereof, may be administered by oral, parenteral (e.g., intramuscular, intraperitoneal, intravenous, CIV, intracisternal injection or infusion, subcutaneous injection, or implant), inhalation, nasal, vaginal, rectal, sublingual, or topical (e.g., transdermal or local) routes of administration. The compound provided herein, or a derivative thereof, may be formulated, alone or together, in suitable dosage unit with pharmaceutically acceptable excipients, carriers, adjuvants and vehicles, appropriate for each route of administration.

In one embodiment, the compound provided herein, or a derivative thereof, is administered orally. In another embodiment, the compound provided herein, or a derivative thereof, is administered parenterally. In yet another embodiment, the compound provided herein, or a derivative thereof, is administered intravenously.

The compound provided herein, or a derivative thereof, can be delivered as a single dose such as, e.g., a single bolus injection, or oral tablets or pills; or over time, such as, e.g., continuous infusion over time or divided bolus doses over time. The compound can be administered repeatedly if necessary, for example, until the subject experiences stable disease or regression, or until the subject experiences disease progression or unacceptable toxicity. For example, stable disease for solid tumors generally means that the perpendicular diameter of measurable lesions has not increased by 25% or more from the last measurement. Response Evaluation Criteria in Solid Tumors (RECIST) Guidelines, Journal of the National Cancer Institute 92(3): 205 216 (2000). Stable disease or lack thereof is determined by methods known in the art such as evaluation of patient symptoms, physical examination, visualization of the tumor that has been imaged using X-ray, CAT, PET, or MRI scan and other commonly accepted evaluation modalities.

The compound provided herein, or a derivative thereof, can be administered once daily (QD), or divided into multiple daily doses such as twice daily (BID), three times daily (TID), and four times daily (QID). In addition, the administration can be continuous (i.e., daily for consecutive days or every day), intermittent, e.g., in cycles (i.e., including days, weeks, or months of rest without drug). As used herein, the term “daily” is intended to mean that a therapeutic compound, such as the compound provided herein, or a derivative thereof, is administered once or more than once each day, for example, for a period of time. The term “continuous” is intended to mean that a therapeutic compound, such as the compound provided herein or a derivative thereof, is administered daily for an uninterrupted period of at least 10 days to 52 weeks. The term “intermittent” or “intermittently” as used herein is intended to mean stopping and starting at either regular or irregular intervals. For example, intermittent administration of the compound provided herein or a derivative thereof is administration for one to six days per week, administration in cycles (e.g., daily administration for two to eight consecutive weeks, then a rest period with no administration for up to one week), or administration on alternate days. The term “cycling” as used herein is intended to mean that a therapeutic compound, such as the compound provided herein or a derivative thereof, is administered daily or continuously but with a rest period. In some such embodiments, administration is once a day for two to six days, then a rest period with no administration for five to seven days.

In some embodiments, the frequency of administration is in the range of about a daily dose to about a monthly dose. In certain embodiments, administration is once a day, twice a day, three times a day, four times a day, once every other day, twice a week, once every week, once every two weeks, once every three weeks, or once every four weeks. In one embodiment, the compound provided herein, or a derivative thereof, is administered once a day. In another embodiment, the compound provided herein, or a derivative thereof, is administered twice a day. In yet another embodiment, the compound provided herein, or a derivative thereof, is administered three times a day. In still another embodiment, the compound provided herein, or a derivative thereof, is administered four times a day.

In certain embodiments, the compound provided herein, or a derivative thereof, is administered once per day from one day to six months, from one week to three months, from one week to four weeks, from one week to three weeks, or from one week to two weeks. In certain embodiments, the compound provided herein, or a derivative thereof, is administered once per day for one week, two weeks, three weeks, or four weeks. In one embodiment, the compound provided herein, or a derivative thereof, is administered once per day for 4 days. In one embodiment, the compound provided herein, or a derivative thereof, is administered once per day for 5 days. In one embodiment, the compound provided herein, or a derivative thereof, is administered once per day for 6 days. In one embodiment, the compound provided herein, or a derivative thereof, is administered once per day for one week. In another embodiment, the compound provided herein, or a derivative thereof, is administered once per day for two weeks. In yet another embodiment, the compound provided herein, or a derivative thereof, is administered once per day for three weeks. In still another embodiment, the compound provided herein, or a derivative thereof, is administered once per day for four weeks.

### VI. METHODS OF TREATMENT

As shown elsewhere herein and well known in the art, IRE1 promotes adaptive remodeling of cellular physiology to alleviate ER stress and enhance cellular proteostasis in response to acute ER insults. Thus, compounds that increase IRE1/XBP1s activity should ameliorate pathologic imbalances in ER proteostasis, and therefore be useful in treatment of diverse diseases. For example, stress-independent activation of a ligand-regulated IRE1 promotes cellular survival in response to chronic chemical ER insults. (Lin, et al. Science 2007, 318 (5852), 944-9) This suggests that IRE1 activation can mitigate ER-stress associated apoptosis implicated in many neurodegenerative diseases. Consistent with this, overexpressing the activated IRE1-regulated transcription factor XBP1s promotes neuroprotection in multiple animal models of neurodegenerative disease including Parkinson's disease, Huntington's disease, and peripheral nerve injury. (Valdes, et al. PNAS 2014, 111(18) 6804-9; Zuleta, et al. Biochem. Biophys. Res. Commun. 2012, 420(3), 558-63; Valenzuela, et al. Cell Death Dis. 2012, 3, e272) Furthermore, stress-independent, chemical genetic activation of IRE1/XBP1s signaling reduces the toxic intracellular aggregation of destabilized, aggregation-prone variants of rhodopsin and al-anti-trypsin (A1AT) implicated in retinitis pigmentosa and Al AT deficiency, respectively. (Shoulders et al. Cell Rep. 2013, 3(4), 1279-92; Chiang et al. Mol. Biol. Cell 2012, 23(5), 758-70; Sifers Proc. Am. Thorac. Soc. 2010, 7(6), 376-80) Increasing XBP1s activity also promotes the degradation of destabilized amyloid precursor protein (APP) mutants, reducing extracellular populations of the APP cleavage product AP that are genetically and pathologically implicated in Alzheimer's disease. (Cui et al. Neurochem. Res. 2018, 43(3), 669-80; Kaneko et al. J. Neurosci. 2010, 30(11), 3924-32) IRE1/XBP1s activation is also advantageous in cellular and animal models of multiple other disorders including diabetes and myocardial infarction, further highlighting the potential for enhancing IRE1 signaling to improve pathologic outcomes in multiple diseases. (Ozcan et al. Science 2004, 306(5695), 457-61; Bi et al. Circ. Res. 2018, 122(11), 1545-54). See also, WO 2021/007594. XBP1 is important for retinal neuronal function. Reduced levels of XBP1 and XBP1s accelerate the decline in retinal function and age-related retinal neurodegeneration in mice (McLaughlin et al. 2018, Molecular Neurodegeneration, 13, 16) and deletion of XBP1 leads to early-onset retinal neurodegeneration in mouse model of Type I diabetes (McLaughlin et al. J. Clin. Med. 2019, 8, 906-920), suggesting a role in diabetic retinopathy (Ma et al. J. Diabetic Res. 2014, Article ID 160140). Deletion of XBP1 from chondrocytes in a mouse model of chondrodysplasia resulted in a dramatic increase in disease severity (shorter limbs, deformed ribcages and disrupted cartilage growth plates), suggesting an important role of XBP1 in response to abnormal protein aggregation in proliferating chondrocytes (Pirog et al. PLoS Genet. 2019, 15, 1008215). Reduced levels of XBP1s have also been associated with cardiovascular diseases. For example, XBP1s levels are reduced in human endomyocardial biopsies in patients with heart failure with preserved ejection fraction (Schiattarella, et al. Nature 2019, 568(7752), 351-356). Overexpression of XBP1s ameliorates the HFpEF phenotype in mice (Schiattarella, supra; Schiattarella, et al. Nat. Commun. 2021, 12, 1684). Reduced levels of XBP1s have also been associated with sensitivity of multiple melanoma cells to the proteasome inhibitor bortezomib (Borjan, et al. Frontiers Onc. 2020, 9, 1530). Over expression of XBP1s has been shown to sensitize multiple melanoma cell lines to bortezomib (Ling, et al. Haematologica 2012, 97(1), 64-72). About one-third of the eukaryotic proteins, including all membrane proteins, enter the endoplasmic reticulum (ER) for their protein folding. Many mutations in ion channel proteins result in their misfolding and the mutant proteins are retained in the ER. Consequently, fewer ion channels reach their working destination. This leads to loss of their function and corresponding disease phenotypes. The ER proteostasis network regulates the ER folding capacity to assure that newly synthesized proteins achieve their native three-dimensional structures in the crowded, oxidative folding environments. The ER proteostasis is mainly monitored by the unfolded protein response (UPR). Examples of such conformational diseases include cystic fibrosis resulting from cystic fibrosis transmembrane conductance regulator (CFTR) misfolding, type 2 long QT syndrome resulting from trafficking deficiency of human ether-à-go-go-related gene (hERG) channels, congenital myasthenic syndromes resulting from misfolding of nicotinic acetylcholine receptors, and idiopathic epilepsy resulting from misfolding of γ-aminobutyric acid type A (GABAA) receptors or sodium channels such as NaV1.1. For example, XBP1s overexpression has been shown to restore trafficking and surface expression of variant of GABAA receptors linked to idiopathic epilepsy (Fu, et al. PLoS ONE 2018, 13(11), e0207948). Osteogenesis Imperfecta (OI) is typically caused by mutations in collagen type-I that disrupt collagen folding and/or stability. XBP1s overexpression increases folding and secretion of variant collagen type-I in primary fibroblast cells of OI patients (DiChiara, et al. bioRxiv 2021, doi.org/10.1101/2021.04.15.439909).

Thus, in another embodiment, provided herein is a method of treating diseases and disorders that may be ameliorated by increasing IRE1/XBP1s activity in a subject by administering to the subject a compound or composition provided herein. In one embodiment, the disease or disorder is a cardiovascular disease, neurodegenerative disease, metabolic disorder, hepatic disorder, protein misfolding disorder or gastrointestinal disorder.

In another embodiment, the disease is a cardiovascular disease, such as myocardial infarction or atherosclerosis.

In other embodiments, the disease is a neurodegenerative disease, including peripheral nerve injury, Creutzfeldt-Jakob disease, Parkinson's disease, and Huntington's disease.

In another embodiment, the disorder is a metabolic disorder, such as diabetes, including type II diabetes, and Gaucher disease.

In another embodiment, the disease is a hepatic disorder, including non-alcoholic fatty liver disease (NAFLD).

In another embodiment, the disorder is a protein misfolding disorder, including amyloid diseases, Alzheimer's disease, ocular diseases such as retinal degeneration, lysosomal storage diseases, and alpha-1 antitrypsin deficiency, including alpha-1 antitrypsin associated emphysema and alpha-1 antitrypsin associated liver disease.

In other embodiments, the disease is an amyloid disease, including atrial amyloidosis, spongiform encephalopathies, senile systemic amyloidosis, hereditary cerebral amyloid angiopathy, familial amyloid polyneuropathy I and II, and familial amyloidosis.

In another embodiment, the disease is a gastrointestinal disorder, including Crohn's disease.

In another embodiment, the disease is retinitis pigmentosa, diabetic retinopathy or retinal neurodegeneration. In another embodiment, the disease is idiopathic epilepsy. In another embodiment, the disease is chondrodysplasia.

### VII. COMBINATION THERAPY WITH A SECOND ACTIVE AGENT

The compound provided herein, or a derivative thereof, can also be combined or used in combination with other therapeutic agents useful in the treatment and/or prevention of diseases and disorders that may be ameliorated by increasing IRE1/XBP1s activity in a subject.

In one embodiment, provided herein is a method of treating, preventing, or managing diseases and disorders that may be ameliorated by increasing IRE1/XBP1s activity in a subject, comprising administering to a subject a compound provided herein, or a pharmaceutically acceptable derivative thereof, in combination with one or more second active agents.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a subject with a disease or disorder. A first therapy (e.g., a prophylactic or therapeutic agent such as a compound provided herein, a compound provided herein, e.g., the compound provided herein, or a derivative thereof) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to the subject. Triple therapy is also contemplated herein.

Administration of the compound provided herein, or a derivative thereof and one or more second active agents to a subject can occur simultaneously or sequentially by the same or different routes of administration. The suitability of a particular route of administration employed for a particular active agent will depend on the active agent itself (e.g., whether it can be administered orally without decomposing prior to entering the blood stream) and the disease or disorder being treated.

The route of administration of the compound provided herein, or a derivative thereof, is independent of the route of administration of a second therapy. In one embodiment, the compound provided herein, or a derivative thereof, is administered orally. In another embodiment, the compound provided herein, or a derivative thereof, is administered intravenously. Thus, in accordance with these embodiments, the compound provided herein, or a derivative thereof, is administered orally or intravenously, and the second therapy can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form. In one embodiment, the compound provided herein, or a derivative thereof, and a second therapy are administered by the same mode of administration, orally or by IV. In another embodiment, the compound provided herein, or a derivative thereof, is administered by one mode of administration, e.g., by IV, whereas the second agent is administered by another mode of administration, e.g., orally.

In one embodiment, the second active agent is administered intravenously or subcutaneously and once or twice daily in an amount of from about 1 to about 1000 mg, from about 5 to about 500 mg, from about 10 to about 350 mg, or from about 50 to about 200 mg. The specific amount of the second active agent will depend on the specific agent used, the type of disease being treated or managed, the severity and stage of disease, and the amount of the compound provided herein, or a derivative thereof, and any optional additional active agents concurrently administered to the subject.

One or more second active ingredients or agents can be used together with the compound provided herein, or a derivative thereof, in the methods and compositions provided herein. Second active agents can be large molecules (e.g., proteins) or small molecules (e.g., synthetic inorganic, organometallic, or organic molecules).

Examples of large molecule active agents include, but are not limited to, hematopoietic growth factors, cytokines, and monoclonal and polyclonal antibodies. Typical large molecule active agents are biological molecules, such as naturally occurring or synthetic or recombinant proteins.

In one embodiment, the compound provided herein, or a derivative thereof, can be administered in an amount ranging from about 0.1 to about 150 mg, from about 1 to about 25 mg, or from about 2 to about 10 mg orally and daily alone, or in combination with a second active agent, prior to, during, or after the use of conventional therapy.

In certain embodiments, second active agents for use herein include other modulators of the UPR, such as but not limited to ATF6 activators and PERK modulators including those disclosed in US 2019/0008809, Fu et al. PLoS ONE 2018, 13(11), e0207948, Fu et al. Science Transl. Med. 2015, 7(292), 292ra98, Ke et al. Cell Death and Disease 2020, 11, 130-143, Halliday 2015 Cell Death and Disease 2015, 6, e1672.

In another embodiment, the second active agent is a pharmacological chaperone, defined as an agent able to bind a protein or protein variant and stabilize it, such as tafamidis (a transthyretin chaperone), 1-deoxygalactonojirimicin (DGJ)(a chaperone of alpha-galactosidase A (AGAL)), SR121463A and VPA-985 (chaperones of the V2 vasopressin receptor), E-4031 (a chaperone of HerG channel) (see, e.g., Liguori et al. International Journal of Molecular Sciences 2020, 21, 489-508, Morello et al. J. Clin. Invest. 2000, 105, 887-895, Zhou et al. J. Biol. Chem. 1999, 274(44), 31123-31126)

### VIII. EXAMPLES

The examples below are meant to illustrate certain embodiments provided herein, and not to limit the scope of this disclosure.

Abbreviations: CDI—carbonyldiimidazole; DCM—Dichloromethane; DIAD Diisopropyl azodicarboxylate; DIEA—diisopropylethylamine; DMAP—4-dimethylaminopyridine; DMF—dimethylformamide; EA—ethyl acetate; h—hour; hrs—hours; HPLC—high pressure liquid chromatography; MPLC—medium pressure liquid chromatography; NMR—nuclear magnetic resonance; PE—petroleum ether; RT—room temperature; TEA- Triethylamine; TBAF—Tetra-n-butylammonium fluoride; THF—tetrahydrofuran; TLC—thin layer chromatography.

### Example 1

### Compound 15: N-(1-(2-(methyl(2-(p-tolyloxy)ethyl)amino)-2-oxoethyl)-1H -pyrazol-4-yl)benzamide

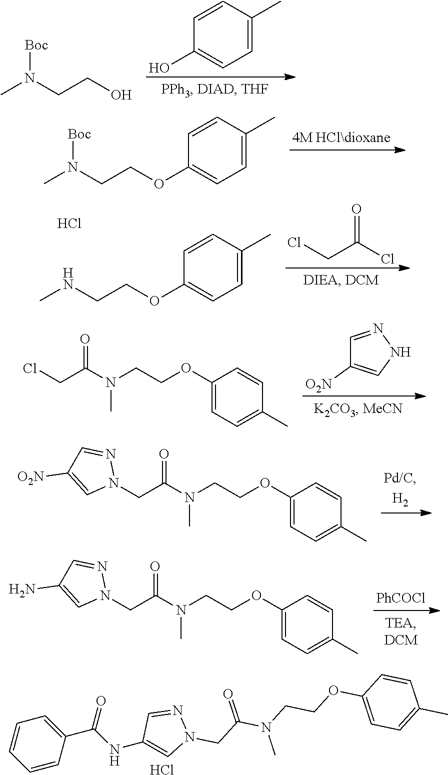

### Step 1: Tert-butyl methyl(2-(p-tolyloxy)ethyl)carbamate

DIAD (1.50 g, 7.42 mmol) was added to the mixture of tert-butyl (2-hydroxyethyl)(methyl)carbamate (1 g, 5.71 mmol), p-cresol (617 mg, 5.71 mmol) and PPh₃ (1.95 g, 7.42 mmol) in THF (10 mL) at 0° C. under N₂, then the mixture was stirred at 25° C. for 16 hrs. The mixture was washed with saturated Na₂CO₃ aqueous (30 mL), then extracted with EtOAc (30 mL×2), the organic phase was washed with brine (50 mL), dried over Na₂SO₄, filtered and the filtrate was concentrated to give the crude product, then purified by column chromatography (SiO₂, PE/EA=20/1 to 10/1) to afford the title compound (0.4 g, 26% yield) as a yellow oil.

¹H NMR (400 MHz, CDCl₃) δ=7.07 (d, J=8.4 Hz, 2H), 6.79 (d, J=8.4 Hz, 2H), 4.06 (s, 2H), 3.58 (s, 2H), 2.97 (s, 3H), 2.28 (s, 3H), 1.46 (s, 9H).

### Step 2: N-Methyl-2-(p-tolyloxy)ethanamine

A solution of tert-butyl methyl(2-(p-tolyloxy)ethyl)carbamate (400 mg, 1.51 mmol) in HCl/dioxane (4 M, 5 mL) was stirred at 25° C. for 0.5 hrs. The mixture was concentrated under reduced pressure to afford the title compound (300 mg, 99% yield, HCl salt) as a yellow solid, which was used without purification.

### Step 3: 2-Chloro-N-methyl-N-(2-(p-tolyloxy)ethyl)acetamide

2-Chloroacetyl chloride (246 mg, 2.18 mmol) was added to a mixture of N-methyl -2-(p-tolyloxy)ethanamine (0.3 g, 1.82 mmol, HCl salt) and DIEA (1.17 g, 9.08 mmol) in DCM (5 mL) at 0° C. under N₂, then the mixture was stirred at 25° C. for 3 hrs. The mixture was quenched with water (5 mL), then extracted with DCM (5 mL×3), the organic phase was washed with brine (10 mL), dried over Na₂SO₄, filtered and the filtrate was concentrated under reduced pressure to give the crude product, then purified by column chromatography (SiO₂, PE/EA=2/1 to 1/1) to afford the title compound (400 mg, 91% yield) as a yellow oil.

¹H NMR (400 MHz, CDCl₃) δ=7.26-7.02 (m, 2H), 6.79-6.73 (m, 2H), 4.27-4.08 (m, 4H), 3.78-3.75 (m, 2H), 3.24-3.04 (m, 3H), 2.29-2.27 (m, 3H)

### Step 4: N-Methyl-2-(4-nitro-1H-pyrazol-1-yl)-N-(2-(p-tolyloxy)ethyl)acetamide

The mixture of 2-chloro-N-methyl-N-(2-(p-tolyloxy)ethyl)acetamide (400 mg, 1.65 mmol), 4-nitro-1H-pyrazole (225 mg, 1.99 mmol) and K₂CO₃ (686 mg, 4.96 mmol) in CH₃CN (5 mL) was stirred at 80° C. for 16 hrs. The mixture was quenched with water (20 mL) and extracted with EtOAc (20 mL×3), the organic phase was washed with brine (30 mL), dried over Na₂SO₄, filtered and the filtrate was concentrated under reduced pressure, then purified by column chromatography (SiO₂, PE/EA=1/1 to 0/1) to afford the title compound (200 mg, 38% yield) as a light yellow oil.

¹H NMR (400 MHz, CDCl₃) δ=8.29 (d, J=2.4 Hz, 1H), 8.08 (d, J=3.6 Hz, 1H), 7.12-7.08 (m, 2H), 6.81-6.76 (m, 2H), 5.24-5.02 (m, 2H), 4.13 (t, J=4.8 Hz, 2H), 3.80-3.76 (m, 2H), 3.25-3.06 (m, 3H), 2.29 (d, J=5.6 Hz, 3H)

### Step 5: 2-(4-amino-1H-pyrazol-1-yl)-N-methyl-N-(2-(p-tolyloxy)ethyl)acetamide

To a solution of N-methyl-2-(4-nitro-1H-pyrazol-1-yl)-N-(2-(p -tolyloxy)ethypacetamide (100 mg, 0.31 mmol) in MeOH (10 mL) was added Pd/C (20 mg, 10% purity) under N₂ atmosphere. The suspension was degassed and purged with H₂ for 3 times. The mixture was stirred under H₂ (50 Psi) at 25° C. for 16 hrs. TLC (PE:EA=1:1). The mixture was filtered by celite and the filtrate was concentrated under reduced pressure to afford the title compound (62 mg, 68% yield, crude) as a brown oil, which was used without purification.

¹H NMR (400 MHz, CDCl₃) δ=7.18-7.17 (m, 1H), 7.14-7.12 (m, 1H), 7.09-7.06 (m, 2H), 6.78-6.75 (m, 2H), 5.03-4.86 (m, 2H), 4.12-4.01 (m, 2H), 3.78-3.73 (m, 2H), 3.16-3.01 (m, 3H), 2.28 (d, J=2.8 Hz, 3H).

Step 6: Benzoyl chloride (35 mg, 0.25 mmol) was added to a solution of 2-(4-amino-1H-pyrazol-1-yl)-N-methyl-N-(2-(p-tolyloxy)ethyl)acetamide (60 mg, 0.21 mmol) and TEA (63 mg, 0.62 mmol) in DCM (2 mL) and stirred at 25° C. for 2 hrs. The mixture was quenched with water (5 mL), then extracted with DCM (5 mL×3), the organic was washed with brine (10 mL), dried over Na₂SO₄, filtered and the filtrate was concentrated under reduced pressure. The product was purified by prep-HPLC (Phenomenex luna C18 150×25 mm×10 μm; [water (HCl)−ACN]; B %: 34-64%) to afford the title compound (32 mg, 39% yield, 99.5% purity) as a white solid.

LCMS: m/z 393.2 [M+H]⁺.

¹H NMR (400 MHz, CD₃OD) δ=8.10 (d, J=3.6 Hz, 1H), 7.93-7.91 (m, 2H), 7.71 (d, J=4.4 Hz, 1H), 7.59-7.49 (m, 3H), 7.12-7.06 (m, 2H), 6.91-6.80 (m, 2H), 5.28-5.15 (m, 2H), 4.18-4.10 (m, 2H), 3.87-3.76 (m, 2H), 3.25 & 3.04 (2 singlets, 3H), 2.25 & 2.27 (2 singlets, 3H).

### Example 2

### Compound 25: N-(1-(2-(Methyl(2-(p-tolyloxy)ethyl)amino)-2-oxo ethyl)-1H -pyrazol-4-yl)-3-phenylpropanamide hydrochloride

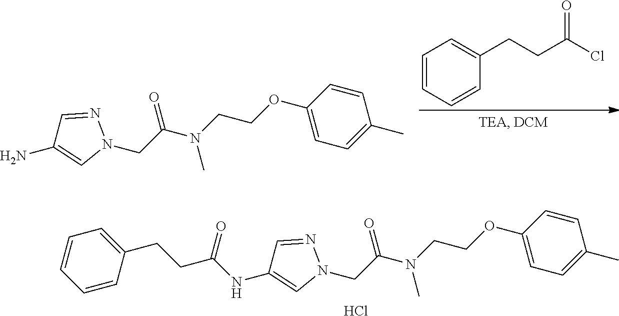 [SMILES: Cc1ccc(OCCN(C)C(=O)Cn2cc(N)cn2)cc1 ; O=C(Cl)CCc1ccccc1 ; Cc1ccc(OCCN(C)C(=O)Cn2cc(NC(=O)CCc3ccccc3)cn2)cc1 ; Cl]

To a solution of 2-(4-amino-1H-pyrazol-1-yl)-N-methyl-N-(2-(p -tolyloxy)ethyl)acetamide (100 mg, 0.35 mmol) in DCM (2 mL) was added TEA (105 mg, 1.04 mmol) and 3-phenylpropanoyl chloride (76 mg, 0.46 mmol) at 0° C. under N₂ and the mixture was stirred at 25° C. for 12 hrs. The mixture was concentrated under reduced pressure and purified by prep-HPLC (Phenomenex luna C18 150×25 mm×10 μm; (water (HCl)-CAN); B %: 36-66%) to afford the title compound (81.85 mg, 51% yield, 99.3% purity, HCl salt) as an off-white solid.

LCMS: m/z 421.3 [M+H]⁺.

¹H NMR (400 MHz, CD₃OD) δ=7.90 (d, J=4.4 Hz, 1H), 7.48 (d, J=3.6 Hz, 1H), 7.28-7.21 (m, 4H), 7.18-7.15 (m, 1H), 7.11-7.05 (m, 2H), 6.88 (d, J=8.4 Hz, 1H), 6.80 (d, J=8.8 Hz, 1H), 5.22 (s, 1H), 5.09 (s, 1H), 4.16-4.08 (m, 2H), 3.84-3.74 (m, 2H), 3.22 (s, 1.5H), 3.02 (s, 1.4H), 2.97 (t, J=7.6 Hz, 2H), 2.62 (t, J=7.2 Hz, 2H), 2.25 & 2.27 (2 singlets, 3H).

### Example 3

### Compound 26: N-Methyl-2-(4-(2-phenylacetamido)-1H-pyrazol-1-yl)-N-(2-(p -tolyloxy)ethypacetamide hydrochloride

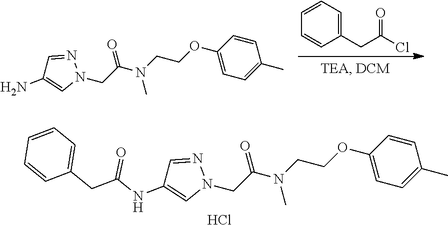 [SMILES: Cc1ccc(OCCN(C)C(=O)Cn2cc(N)cn2)cc1 ; O=C(Cl)Cc1ccccc1 ; Cc1ccc(OCCN(C)C(=O)Cn2cc(NC(=O)Cc3ccccc3)cn2)cc1 ; Cl]

To a solution of 2-(4-amino-1H-pyrazol-1-yl)-N-methyl-N-(2-(p -tolyloxy)ethyl)acetamide (100 mg, 0.35 mol) in DCM (2 mL) was added TEA (105 mg, 1.04 mmol) and 2-phenylacetyl chloride (70 mg, 0.45 mmol) at 0° C. under N₂ and the mixture was stirred at 25° C. for 12 hrs. The reaction mixture was concentrated under reduced pressure to remove DCM. The residue was diluted with H₂O (15 mL) and extracted with EtOAc (20 mL×3). The combined organic layers were washed with brine (10 mL×3), dried over Na₂SO₄, filtered and concentrated under reduced pressure. The residue was purified by prep-HPLC (Phenomenex luna C18 150×25 mm×10 μm; (water (HCl)-CAN); B %: 31-61%) to afford the title compound (63.47 mg, 40% yield, 97.6% purity, HCl salt) as a white solid.

LCMS: m/z 407.3 [M+H]⁺.

¹H NMR (400 MHz, DMSO-d₆) δ=10.16 (s, 1H), 7.82 (d, J=1.6 Hz, 1H), 7.41 (d, J=4.0 Hz, 1H), 7.34-7.21 (m, 5H), 7.11-7.06 (m, 2H), 6.88-6.81 (m, 2H), 5.15 (s, 1H), 5.06 (s, 1H), 4.12-4.00 (m, 2H), 3.74 (bt, J=5.2 Hz, 1H), 3.62 (bt, J=5.6 Hz, 1H), 3.56 (s, 2H), 3.09 (s, 1.5H), 2.89 (s, 1.5H), 2.23 & 2.22 (2 singlets, 3H).

### Example 4

### Compound 27: N-(1-(2-(methyl(phenethyl)amino)-2-oxoethyl)-1H-pyrazol-4-yl) -3-phenoxypropanamide hydrochloride

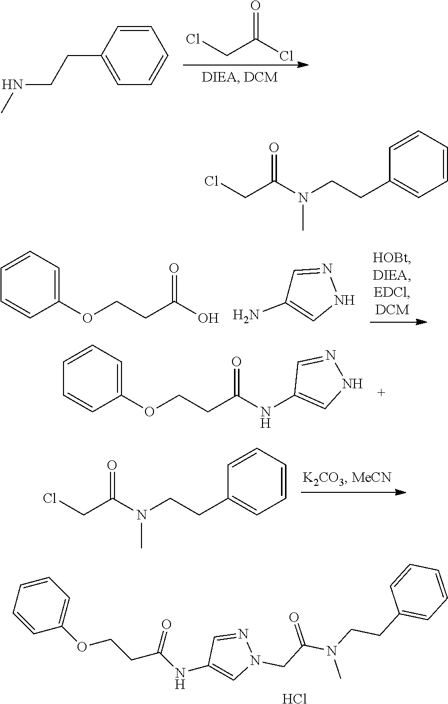

### Step 1: 3-Phenoxy-N-(1H-pyrazol-4-yl)propanamide

To a solution of 3-phenoxypropanoic acid (2.0 g, 12.03 mmol) in DCM (30 mL) was added EDCI (2.54 g, 13.24 mmol) and HOBt (1.71 g, 12.64 mmol) at 0° C. and stirred for 1 h. A solution of 1H-pyrazol-4-amine (1 g, 12.03 mmol) and DIEA (4.67 g, 36.10 mmol, 6.29 mL) in DCM (20 mL) was added to the mixture dropwise at 0° C. and the mixture was stirred at 25° C. for 12 hrs. The reaction mixture was concentrated under reduced pressure to remove DCM. The residue was diluted with H₂O (40 mL) and extracted with EtOAc (30 mL×3). The combined organic layers were washed with brine (20 mL×3), dried over Na₂SO₄, filtered and concentrated under reduced pressure. The crude product was purified by column chromatography (SiO₂, PE/EA=10/1 to PE/R=3/1, R=(EtOAc/EtOH=3/1) to afford the title compound (1.2 g, 43% yield) as a white solid.

LCMS: m/z 232.1 [M+H]⁺.

### Step 2: 2-Chloro-N-methyl-N-phenethylacetamide

To a solution of N-methyl-2-phenylethanamine (1 g, 7.40 mmol, 1.08 mL) in DCM (40 mL) was added DIEA (1.91 g, 14.79 mmol) and 2-chloroacetyl chloride (1.09 g, 9.61 mmol) at 0° C. and the mixture was stirred at 25° C. for 12 hrs. The reaction mixture was concentrated under reduced pressure and the residue was purified by column chromatography (SiO₂, PE/EA=100/1 to 3/1) to afford the title compound (800 mg, 51% yield) obtained as a yellow oil.

¹H NMR (400 MHz, CDCl₃) δ=7.37-7.18 (m, 5H), 4.08 (s, 1H), 3.70 (s, 1H), 3.65-3.58 (m, 2H), 3.03 (s, 1.5H), 2.96 (s, 1.5H), 2.94-2.88 (m, 2H)

### Step 3: N-(1-(2-(Methyl(phenethyl)amino)-2-oxoethyl)-1H-pyrazol-4-yl)-3-phenoxypropanamide hydrochloride

To a solution of 3-phenoxy-N-(1H-pyrazol-4-yl)propanamide (100 mg, 0.43 mmol) in MeCN (10 mL) was added K₂CO₃ (120 mg, 0.86 mmol) and 2-chloro-N-methyl-N -phenethylacetamide (110 mg, 519 mmol) and the mixture was stirred at 80° C. for 16 hrs. The reaction mixture was concentrated under reduced pressure. The residue was diluted with H₂O (20 mL) and extracted with EtOAc (15 mL×3). The combined organic layers were washed with brine (10 mL×3), dried over Na₂SO₄, filtered and concentrated under reduced pressure. The residue was purified by prep-HPLC (Phenomenex luna C18 150×25 mm×10 μm; (water (HCl)-CAN); B %: 33-63%) to afford the title compound (89.1 mg, 45% yield, 97% purity, HCl salt) as a white solid.

LCMS: m/z 407.2 [M+H]⁺.

¹H NMR (400 MHz, DMSO-d₆) δ=10.10 (d, J=6.4 Hz, 1H), 7.84-7.70 (m, 1H), 7.42-7.39 (m, 1H), 7.35-7.18 (m, 7H), 6.94-6.91 (m, 3H), 5.02 (s, 1H), 4.77 (s, 1H), 4.26-4.23 (m, 2H), 3.56-3.45 (m, 2H), 2.96 (s, 1.5H), 2.85-2.82 (m, 2.5H), 2.76-2.67 (m, 3H).

### Example 5

### Compound 28: N-(1-(2-(Methyl(phenyl)amino)-2-oxoethyl)-1H-pyrazol-4-yl)-3-phenoxypropanamide hydrochloride

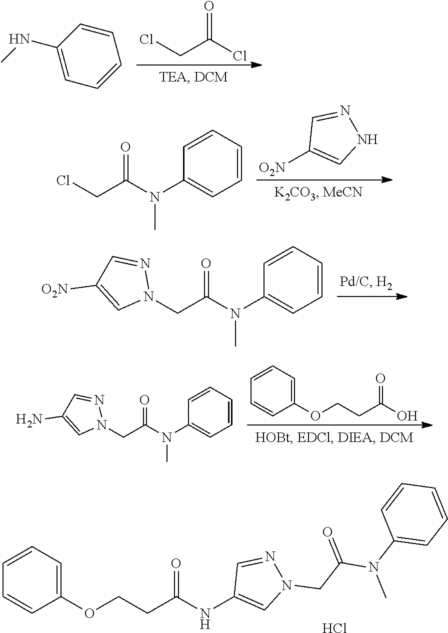

### Step 1: 2-Chloro-N-methyl-N-phenylacetamide

To a solution of N-methylaniline (1 g, 9.33 mmol) in DCM (40 mL) was added DIEA (2.41 g, 18.66 mmol) and 2-chloroacetyl chloride (1.26 g, 11.20 mmol) at 0° C. and the mixture was stirred at 25° C. for 12 hrs. The reaction mixture was concentrated under reduced pressure. The residue was purified by column chromatography (SiO₂, PE:EA=100:1 to 3:1) to afford the title compound (700 mg, 41% yield) as a yellow oil.

¹H NMR (400 MHz, CDCl₃) δ=7.47-7.37 (m, 3H), 7.26-7.24 (m, 2H), 3.85 (s, 2H), 3.32 (s, 3H).

### Step 2: N-Methyl-2-(4-nitro-1H-pyrazol-1-yl)-N-phenylacetamide

To a solution of 2-chloro-N-methyl-N-phenylacetamide (350 mg, 1.91 mmol) in MeCN (30 mL) was added K₂CO₃ (527 mg, 3.81 mmol) and 4-nitro-1H-pyrazole (258 mg, 2.29 mmol) and the mixture was stirred at 80° C. for 16 hrs. The reaction mixture was filtered and the filtrate concentrated under reduced pressure to afford the title compound (800 mg, crude) as a yellow oil.

LCMS: m/z 261.1 [M+H]⁺.

### Step 3: 2-(4-Amino-1H-pyrazol-1-yl)-N-methyl-N-phenylacetamide

To a solution of N-methyl-2-(4-nitro-1H-pyrazol-1-yl)-N-phenylacetamide (300 mg, 1.15 mmol) in EtOH (25 mL) was added Pd/C (30 mg, 10% purity). The suspension was degassed under reduced pressure and purged with H₂ three times. The reaction mixture was stirred under H₂ balloon (15 Psi) at 25° C. for 12 hrs. The reaction mixture was filtered and the filtrate concentrated under reduced pressure to afford the title compound (250 mg, crude) as a yellow oil.

LCMS: m/z 231.2 [M+H]⁺.

### Step 4: N-(1-(2-(Methyl(phenyl)amino)-2-oxoethyl)-1H-pyrazol-4-yl)-3-phenoxypropanamide hydrochloride

To a solution of 3-phenoxypropanoic acid (100 mg, 0.60 mmol) in DCM (5 mL) was added EDCI (127 mg, 0.66 mmol) and HOBt (85 mg, 0.63 mmol) at 0° C. and stirred for 1 h. 2-(4-amino-1H-pyrazol-1-yl)-N-methyl-N-phenylacetamide (150 mg, 0.65 mmol) and DIEA (233 mg, 1.81 mmol) were added at 0° C. and the mixture was stirred at 25° C. for 12 hrs. The reaction mixture was concentrated under reduced pressure to remove DCM. The residue was diluted with H₂O (20 mL) and extracted with EtOAc (15 mL×3). The combined organic layers were washed with brine (10 mL×3), dried over Na₂SO₄, filtered and concentrated under reduced pressure. The residue was purified by prep-HPLC (Phenomenex luna C18 150×25 mm×10 μm; (water (HCl)-CAN); B %: 29-59%) to afford the title compound (46.95 mg, 20% yield, 98% purity) as a pink solid.

LCMS: m/z 379.1 [M+H]⁺.

¹H NMR (400 MHz, DMSO-d₆) δ=10.10 (s, 1H), 7.84 (s, 1H), 7.50-7.39 (m, 6H), 7.29-7.25 (m, 2H), 6.94-6.91 (m, 3H), 4.65 (bs, 2H), 4.23 (bt, J=6.0 Hz, 2H), 3.19 (bs, 3H), 2.70 (bt, J=6.0 Hz, 2H).

### Example 6

### Compound 29: N-(1-(2-(Methyl(2-phenoxyethyl)amino)-2-oxoethyl)-1H-pyrazol -4-yl)-3-phenoxypropanamide hydrochloride

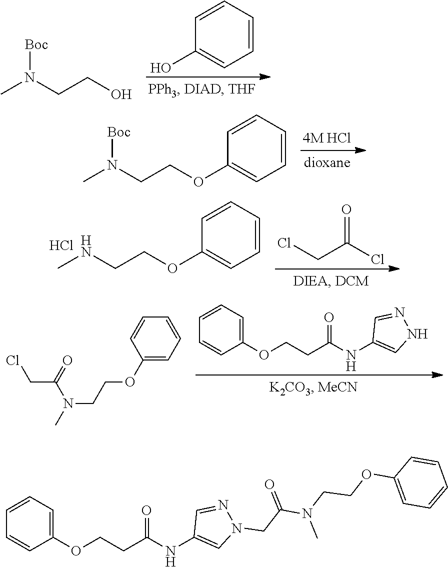

### Step 1: Tert-butyl methyl(2-phenoxyethyl)carbamate

To a solution of tert-butyl (2-hydroxyethyl)(methyl)carbamate (1 g, 5.71 mmol) in THF (30 mL) was added PPh₃ (2.25 g, 8.56 mmol) and phenol (806 mg, 8.56 mmol) at 25° C., and then DIAD (1.73 g, 8.56 mmol) was added to the mixture under N₂ at 0° C. The reaction mixture was heated at 80° C. and stirred for 16 hrs. The reaction mixture was concentrated under reduced pressure. The residue was diluted with H₂O (40 mL) and extracted with EtOAc (20 mL×3). The combined organic layers were washed with brine (20 mL×3), dried over Na₂SO₄, filtered and concentrated under reduced pressure. The residue was purified by column chromatography (SiO₂, PE:EA=1:0 to 20:1) to afford the title compound (1.7 g, crude) as a yellow oil.

LCMS: m/z 152.2 [M+H−100]⁺.

¹H NMR (400 MHz, CDCl₃) δ=7.30-7.28 (m, 1H), 7.25-7.21 (m, 1H), 6.97-6.92 (m, 1H), 6.88-6.82 (m, 2H), 4.09 (s, 2H), 3.60 (bs, 2H), 2.99 (s, 3H), 1.47 (s, 9H)

### Step 2: N-Methyl-2-phenoxyethanamine hydrochloride

Tert-butyl methyl(2-phenoxyethyl)carbamate (900 mg, 3.58 mmol) was dissolved in 4M HCl/dioxane (20 mL) and stirred at 25° C. for 16 hrs. The reaction mixture was concentrated under reduced pressure. The residue was purified by slurry in EtOAc (60 mL) at 25° C. for 2 h. The title compound (420 mg, 62% yield, HCl salt) was filtered off as a white solid.

LCMS: m/z 151.7 [M+H]⁺.

¹H NMR (400 MHz, DMSO-d₆) δ=9.19 (bs, 2H), 7.35-7.30 (m, 2H), 7.00-6.67 (m, 3H), 4.26 (t, J=4.8 Hz, 2H), 3.30 (bt, J=5.2 Hz, 2H), 2.60 (s, 3H).

### Step 3: 2-Chloro-N-methyl-N-(2-phenoxyethyl)acetamide

To a solution of N-methyl-2-phenoxyethanamine hydrochloride (100 mg, 0.53 mmol, HCl salt) in DCM (5 mL) was added DIEA (344 mg, 2.66 mmol) and 2-chloroacetyl chloride (78 mg, 0.69 mmol) at 0° C. and the mixture was stirred at 25° C. for 16 hrs. The reaction mixture was concentrated under reduced pressure. The residue was purified by column chromatography (SiO₂, PE:EA=100:1 to 3:1) to afford the title compound (80 mg, 66% yield) as a yellow oil.

LCMS: m/z 228.1 [M+H]⁺.

¹H NMR (400 MHz, CDCl₃) δ=7.34-7.30 (m, 2H), 7.05-6.88 (m, 3H), 4.30 (s, 1H), 4.20-4.16 (m, 2H), 4.12 (s, 1H), 3.83-3.79 (m, 2H), 3.27-3.07 (m, 3H)

### Step 4: N-(1-(2-(Methyl(2-phenoxyethyl)amino)-2-oxoethyl)-1H-pyrazol-4-yl)-3-phenoxypropanamide hydrochloride

To a solution of 3-phenoxy-N-(1H-pyrazol-4-yl)propanamide (74 mg, 0.32 mmol) in MeCN (20 mL) was added K₂CO₃ (88 mg, 0.64 mmol) and 2-chloro-N-methyl-N-(2-phenoxyethyl)acetamide (80 mg, 0.35 mmol) and the mixture was stirred at 80° C. for 16 hrs. The reaction mixture was concentrated under reduced pressure. The residue was purified by prep-HPLC (Phenomenex luna C18 150×25 mm×10 μm; (water (HCl)-CAN); B %: 33-63%) to afford the title compound (36 mg, 23% yield, 95% purity, HCl salt) as an off-white solid.

LCMS: m/z 423.3 [M+H]⁺.

¹H NMR (400 MHz, DMSO-d₆) δ=10.10 (d, J=1.2 Hz, 1H), 7.86 (d, J=3.6 Hz, 1H), 7.42 (d, J=5.6 Hz, 1H), 7.33-7.26 (m, 4H), 7.00-6.91 (m, 6H), 5.18 & 5.08 (2 singlets, 2H), 4.24 (t, J=6.0 Hz, 2H), 4.16 (t, J=5.2 Hz, 1H), 4.07 (t, J=5.6 Hz, 1H), 3.78 (t, J=5.2 Hz, 1H), 3.65 (t, J=5.6 Hz, 1H), 3.11 (s, 1.5H), 2.90 (s, 1.5H), 2.71 (t, J=6.0 Hz, 2H).

### Example 7

### Compound 30: N-(1-(2-(Methyl(2-(4-(trifluoromethyl)phenoxy)ethyl)amino)-2-oxoethyl)-1H-pyrazol-4-yl)-3-phenoxypropanamide hydrochloride

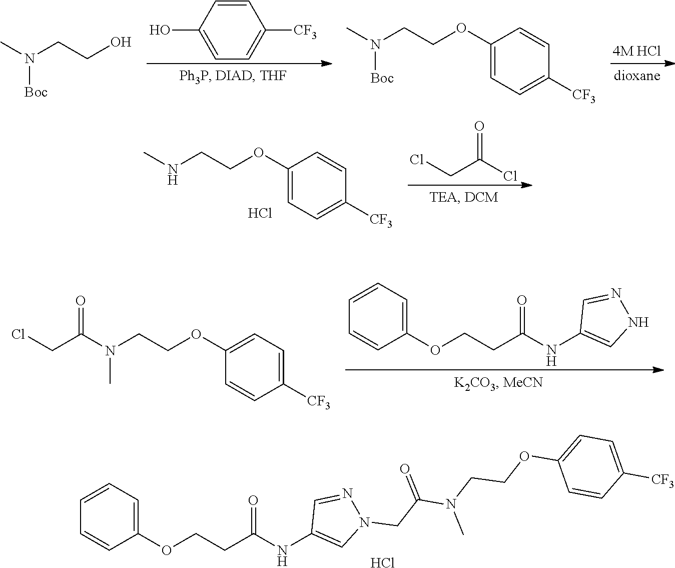

### Step 1: Tert-butyl methyl(2-(4-(trifluoromethyl)phenoxy)ethyl)carbamate

To a solution of tert-butyl (2-hydroxyethyl)(methyl)carbamate (2 g, 11.41 mmol) in THF (40 mL) was added PPh₃ (4.49 g, 17.12 mmol) and 4-(trifluoromethyl)phenol (2.04 g, 12.56 mmol) at 25° C., and then DIAD (3.46 g, 17.12 mmol) was added to the mixture under N₂ at 0° C. The reaction mixture was heated at 80° C. and stirred for 16 hrs. The reaction mixture was concentrated under reduced pressure. The residue was diluted with H₂O (40 mL) and extracted with EtOAc (30 mL×3). The combined organic layers were washed with brine (15 mL×3), dried over Na₂SO₄, filtered and concentrated under reduced pressure. The residue was purified by column chromatography (SiO₂, PE/EA=100/1 to 10/1). The crude product was purified by prep-MPLC (Welch Ultimate XB_C18 20-40 um; [H₂O+ACN]; B %: 55%) to afford the title compound (700 mg, 17% yield) as a black brown oil.

LCMS: m/z 220.1 [M+H−100]⁺.

¹H NMR (400 MHz, DMSO-d₆) δ=7.64 (d, J=8.8 Hz, 2H), 7.13 (d, J=8.8 Hz, 2H), 4.18 (bs, 2H), 3.55 (t, J=5.2 Hz, 2H), 2.85 (bs, 3H), 1.39-1.33 (m, 9H)

### Step 2: N-Methyl-2-(4-(trifluoromethyl)phenoxy)ethanamine hydrochloride

A solution of tert-butyl methyl(2-(4-(trifluoromethyl)phenoxy)ethyl)carbamate (700 mg, 1.92 mmol) in HCl/dioxane (4 M, 25 mL) was stirred at 25° C. for 16 hrs. The reaction mixture was concentrated under reduced pressure to afford the title compound (600 mg, crude, HCl salt) as a yellow solid.

¹H NMR (400 MHz, DMSO-d₆) δ=9.23 (bs, 2H), 7.69 (bd, J=8.4 Hz, 2H), 7.18 (bd, J=8.8 Hz, 2H), 4.36 (t, J=4.8 Hz, 2H), 3.34-3.29 (m, 2H), 2.61 (bs, 3H)

### Step 3: 2-Chloro-N-methyl-N-(2-(4-(trifluoromethyl)phenoxy)ethyl)acetamide

To a solution of N-methyl-2-(4-(trifluoromethyl)phenoxy)ethanamine hydrochloride (200 mg, 0.78 mmol, HCl salt) in DCM (10 mL) was added DIEA (202 mg, 1.56 mmol) and 2-chloroacetyl chloride (115 mg, 1.02 mmol) at 0° C. and stirred at 25° C. for 12 hrs. The reaction mixture was concentrated under reduced pressure. The residue was purified by chromatography (SiO₂, PE/EA=100/1 to 1/1) to afford the title compound (160 mg, 69% yield) as a yellow oil.

LCMS: m/z 296.0 [M+H]⁺.

¹H NMR (400 MHz, DMSO-d₆) δ=7.68-7.64 (m, 2H), 7.14-7.12 (m, 2H), 4.48 (s, 1H), 4.40 (s, 1H), 4.24 (t, J=5.2 Hz, 1H), 4.18 (t, J=5.6 Hz, 1H), 3.77 (t, J=5.6 Hz, 1H), 3.69 (t, J=5.6 Hz, 1H), 3.11 (s, 1.5H), 2.92 (s, 1.5H)

### Step 4: N-(1-(2-(Methyl(2-(4-(trifluoromethyl)phenoxy)ethyl)amino)-2-oxoethyl) -1H-pyrazol-4-yl)-3-phenoxypropanamide hydrochloride

To a solution of 3-phenoxy-N-(1H-pyrazol-4-yl)propanamide (100 mg, 0.43 mmol) in MeCN (30 mL) was added 2-chloro-N-methyl-N-(2-(4-(trifluoromethyl)phenoxy)ethyl)acetamide (153 mg, 0.52 mmol) and K₂CO₃ (119 mg, 0.87 mmol) and the mixture was stirred at 80° C. for 16 hrs. The reaction mixture was concentrated under reduced pressure. The residue was diluted with H₂O (20 mL) and extracted with EtOAc (20 mL×3). The combined organic layers were washed with brine (10 mL×3), dried over Na₂SO₄, filtered and concentrated under reduced pressure. The residue was purified by prep-HPLC (Phenomenex luna C18 150×25 mm×10 μm; (water (HCl)-CAN); B %: 39-69%) to afford the title compound (138.67 mg, 58.0% yield, 95.3% purity, HCl salt) as a white solid.

LCMS: m/z 491.1 [M+H]⁺.

¹H NMR (400 MHz, DMSO-d₆) δ=10.09 (d, J=1.6 Hz, 1H), 7.87 (d, J=10.0 Hz, 1H), 7.69-7.64 (m, 2H), 7.42 (d, J=8.0 Hz, 1H), 7.30-7.25 (m, 2H), 7.20-7.12 (m, 2H), 6.94-6.91 (m, 3H), 5.19 & 5.08 (2 singlets, 2H), 4.27-4.23 (m, 3H), 4.17 (t, J=5.6 Hz, 1H), 3.81 (bt, J=5.2 Hz, 1H), 3.67 (t, J=5.6 Hz, 1H), 3.12 (s, 1.6H), 2.91 (s, 1.4H), 2.71 (bt, J=6.0 Hz, 2H).

### Example 8

### Compound 40: N-(1-(2-(methyl(2-(4-(3-(trifluoromethyl)-3H-diazirin-3yl)phenoxy)ethypamino)-2-oxoethyl)-1H-pyrazol-4-yl)-3-phenoxypropanamide

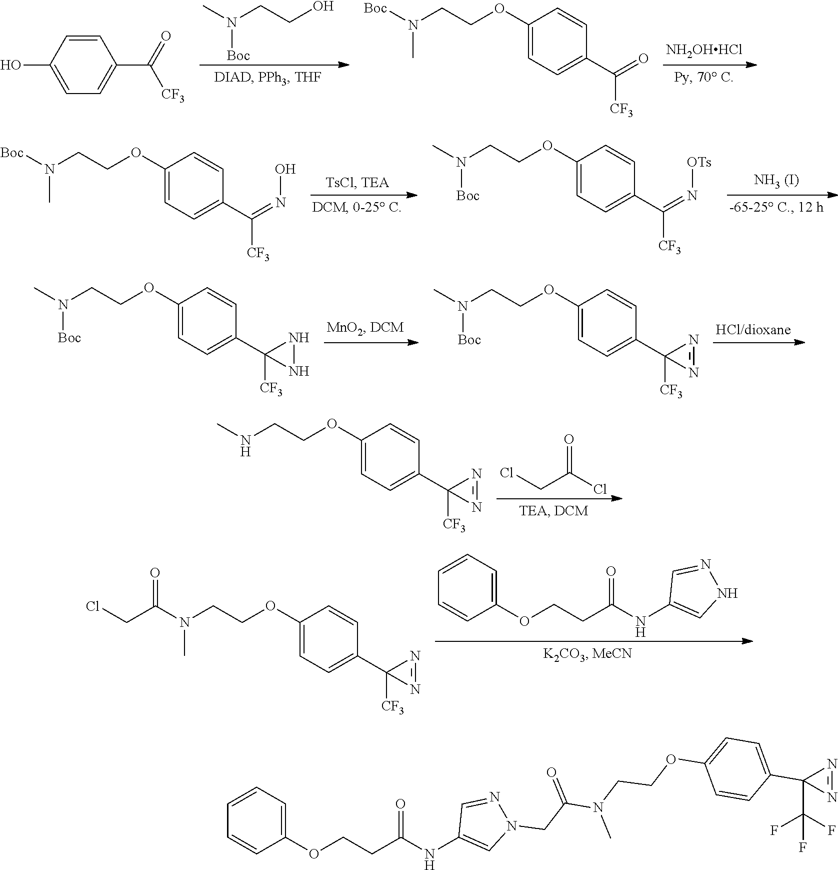

### Step 1: Tert-butyl methyl(2-(4-(2,2,2-trifluoroacetyl)phenoxy)ethyl)carbamate

To a solution of 2,2,2-trifluoro-1-(4-hydroxyphenyl)ethanone (5 g, 26.30 mmol) and tert-butyl N-(2-hydroxyethyl)-N-methyl-carbamate (6.91 g, 39.45 mmol) in THF (125 mL) was added PPh₃ (8.28 g, 31.56 mmol), followed by DIAD (7.98 g, 39.45 mmol) at 0° C. under N₂. The mixture was stirred for 16 hrs at 25° C. The mixture was concentrated under reduced pressure. The residue was purified by flash silica gel chromatography (40 g Silica Flash Column, eluent of 0˜20% EA/PE gradient@40 mL/min) to afford the title compound (4.8 g, 53% yield) as yellow oil.

LCMS: m/z 292.2 [M+H−56]⁺

### Step 2: Tert-butyl (Z)-(2-(4-(2,2,2-trifluoro-1-(hydroxyimino)ethyl)phenoxy)ethyl)carbamate

To a solution of tert-butyl methyl(2-(4-(2,2,2-trifluoroacetyl)phenoxy)ethyl)carbamate (4.8 g, 13.82 mmol) in pyridine (50 mL) was added NH₂OH·HCl (1.15 g, 16.58 mmol) at 25° C. The mixture was quenched by H₂O (20 mL) and concentrated under reduced pressure. Water (10 mL) was added, and the aqueous mixture was extracted with ethyl acetate (20 mL×3). The combined organic phase was washed with brine (30 mL), dried over anhydrous sodium sulfate, filtered, and concentrated under reduced pressure to afford the title compound (7.16 g, crude) as yellow oil, which was used in next step without purification.

LCMS: m/z 263.1 [M+H−100]⁺

### Step 3: Tert-butyl (Z)-(2-(4-(2,2,2-trifluoro-1-((tosyloxy)imino)ethyl)phenoxy)ethyl)carbamate

To a solution of tert-butyl (Z)-(2-(4-(2,2,2-trifluoro-1-(hydroxyimino)ethyl)phenoxy)ethyl)carbamate (7.16 g, 19.76 mmol) in DCM (30 mL) was added TEA (4.0 g, 39.52 mmol) and TsCl (4.52 g, 23.71 mmol) at 0° C. The reaction mixture was stirred for 2 h at 25° C. Water (30 mL) was added. The resulting mixture was extracted with DCM (50 mL×3). The combined organic phase was washed with brine (30 mL), dried over anhydrous sodium sulfate, filtered, and concentrated under reduced pressure to afford the title compound (4.65 g, crude) as orange gum, which was used in next step without purification.

LCMS: m/z 417.0 [M+H−100]⁺

### Step 4: tert-butyl methyl(2-(4-(3-(trifluoromethyl)diaziridin-3-yl)phenoxy)ethyl)carbamate

Tert-butyl (Z)-(2-(4-(2,2,2-trifluoro-1-((tosyloxy)imino)ethyl)phenoxy)ethyl)carbamate (4.56 g, 8.83 mmol) was added to NH₃(1) (50 mL) in a sealed tube at −65° C. The mixture was warmed to 25° C. and stirred for 16 hrs. The suspension was filtered and the filtrated was concentrated under reduced pressure. The residue was purified by silica gel column chromatography (PE:EA=5:1 to 2:1) to afford the title compound (2.82 g, 88% yield) as yellow solid.

LCMS: m/z 262.0 [M+H−100]⁺

¹H NMR (400 MHz, CDCl₃) δ=7.51 (d, J=6.4 Hz, 2H), 6.89 (d, J=13.6 Hz, 2H), 4.11 (t, J=7.2 Hz, 2H), 3.67-3.52 (m, 2H), 1.45 (s, 9H).

### Step 5: Tert-butyl methyl(2-(4-(3-(trifluoromethyl)-3H-diazirin-3-yl)phenoxy)ethyl)carbamate

To a solution of tert-butyl methyl(2-(4-(3-(trifluoromethyl)diaziridin-3-yl)phenoxy)ethyl)carbamate (2.82 g, 7.80 mmol) in DCM (30 mL) was added MnO₂ (6.78 g, 78.04 mmol) at 25° C. The reaction mixture was stirred for 16 hrs at 25° C. The suspension was filtered and the filtrated was concentrated under reduced pressure to afford the title compound (2.8 g, 99% yield) as yellow oil, which was used into next step without purification

LCMS: m/z 304.0 [M+H−56]⁺

### Step 6: N-methyl-2-(4-(3-(trifluoromethyl)-3H-diazirin-3-yl)phenoxy)ethan-1-amine

To a solution of tert-butylmethyl(2-(4-(3-(trifluoromethyl)-3H-diazirin-3-yl)phenoxy)ethyl)carbamate (2.8 g, 7.79 mmol) in DCM (10 mL) was added HCl/dioxane (10 mL), then the mixture was stirred at 25° C. for 2 hrs. The mixture was concentrated under reduced pressure. The residue was purified by trituration with MTBE (10 mL) at 25° C. for 0.5 h. After filtration and drying under vacuum, the title compound (120 mg, 5% yield, HCl) was obtained as a brown solid. The filtrate was concentrated.

LCMS: m/z 260.0 [M+H]⁺

### Step 7: 2-chloro-N-methyl-N-(2-(4-(3-(trifluoromethyl)-3H-diazirin-3-yl)phenoxy)ethypacetamide

To a solution of N-methyl-2-(4-(3-(trifluoromethyl)-3H-diazirin-3-yl)phenoxy)ethan-1-amine (50 mg, 0.17 mmol, HCl salt) and TEA (43 mg, 0.42 mmol) in DCM (1 mL) was added 2-chloroacetyl chloride (25 mg, 0.22 mmol) at 0° C. The reaction mixture was stirred for 16 h at 25° C. Water (5 mL) was added. The resulting mixture was extracted with ethyl acetate (10 mL×3). The combined organic phase was washed with brine (5 mL), dried over anhydrous sodium sulfate, filtered, and concentrated under reduced pressure to afford the title compound (60 mg, crude) as yellow gum, which was used into next step without purification.

LCMS: m/z 335.9 [M+H]⁺

### Step 8: N-(1-(2-(methyl(2-(4-(3-(trifluoromethyl)-3H-diazirin-3-yl)phenoxy)ethypamino)-2-oxoethyl)-1H-pyrazol-4-yl)-3-phenoxypropanamide

To a solution of 2-chloro-N-methyl-N-(2-(4-(3-(trifluoromethyl)-3H-diazirin-3-yl)phenoxy)ethypacetamide (60 mg, 0.18 mmol) in DMF (1.5 mL) was added K₂CO₃ (74 mg, 0.54 mmol), followed by 3-phenoxy-N-(1H-pyrazol-4-yl)propanamide (45 mg, 0.20 mmol) at 25° C. The reaction mixture was stirred for 32 hrs at 25° C. The suspension was filtered and the filtrated was concentrated under reduced pressure. The residue was purified by prep-HPLC(column: Waters×bridge 150×25 mm 10 μm; mobile phase: (water (NH₄HCO₃)-CAN); B %: 40%-70%, 8 min) and prep-HPLC(column: YMC-Pack CN 150×30 mm×5 μm; mobile phase: (Hexane-EtOH); B %: 13%-32%, 9 min) to afford the title compound (13.34 mg, 13% yield, 91.3% purity) as yellow solid.

LCMS: m/z 531.2 [M+H]⁺

¹H NMR (400 MHz, CD₃OD) δ=7.96 (d, J=4.4 Hz, 1H), 7.54 (d, J=6.0 Hz, 1H), 7.29-7.16 (m, 4H), 7.09 (d, J=8.8 Hz, 1H), 7.03-6.98 (m, 1H), 6.96-6.85 (m, 3H), 5.25-5.08 (m, 2H), 4.35-4.27 (m, 2H), 4.23-4.13 (m, 2H), 3.87-3.74 (m, 2H), 3.21-3.11 (m, 2H), 3.05-2.96 (m, 2H), 2.78 (dt, J=2.8, 6.0 Hz, 2H).

### Example 9

### Compound 50: N-(1-(2-(methyl(2-(p-tolyloxy)ethyl)amino)-2-oxoethyl)-1H -pyrazol-3-yl)-3-phenoxypropanamide

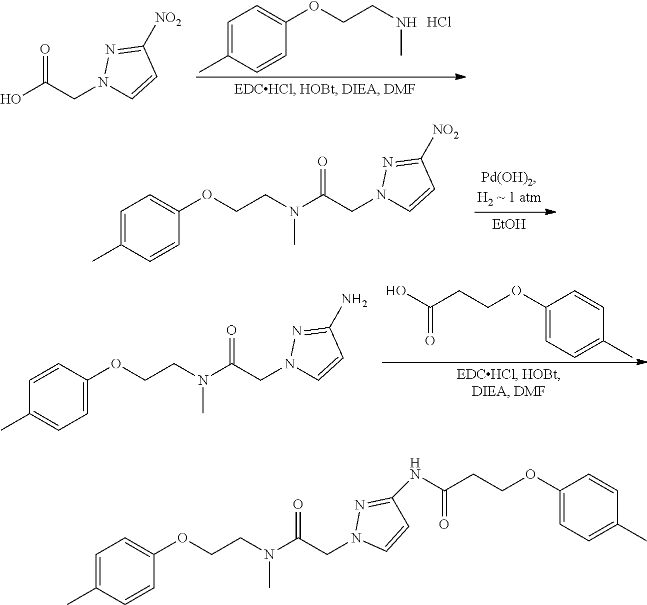

### Step 1: N-methyl-2-(3-nitro-1H-pyrazol-1-yl)-N-(2-(p-tolyloxy)ethyl)acetamide

To a suspension of (3-nitro-1H-pyrazol-1-yl)acetic acid (250 mg, 1.46 mmol), HOBt hydrate (366 mg, 2.192 mmol), and EDC HCl (420 mg, 2.192 mmol) stirring under N₂ in DMF (6 mL) at 25° C. was added DIEA (827 μL, 4.748 mmol). The reaction mixture was then allowed to stir briefly before adding N-methyl-2-(p-tolyloxy)ethan-1-amine hydrochloride (368 mg, 1.826 mmol). Stirring was continued at 25° C. for the following 18 hrs. The reaction mixture was then concentrated under reduced pressure and the resultant residue then purified by column chromatography (SiO₂, MeOH:DCM—0:1 to 1:9) to afford the title compound (442.5 mg) as a pale off-white solid.

LCMS: m/z 319.1 [M+H]⁺.

¹H NMR (600 MHz, DMSO-d₆) δ 7.97 (d, J=2.6 Hz, 0.5H), 7.93 (d, J=2.6 Hz, 0.5H), 7.13-7.05 (m, 4H), 6.92 (d, J=8.5 Hz, 0.5H), 6.83 (d, J=8.5 Hz, 0.5H), 5.44 (s, 1H), 5.37 (s, 1H), 4.15 (t, J=5.2 Hz, 1H), 4.04 (t, J=5.7 Hz, 1H), 3.78 (t, J=5.2 Hz, 1H), 3.65 (t, J=5.7 Hz, 1H), 3.13 (s, 1.5H), 2.92 (s, 1.5H), 2.23 (ds, J=9.2 Hz, 3H).

### Step 2: 2-(3-amino-1H-pyrazol-1-yl)-N-methyl-N-(2-(p-tolyloxy)ethyl)acetamide

To a suspension of N-methyl-2-(3-nitro-1H-pyrazol-1-yl)-N-(2-(p -tolyloxy)ethypacetamide (442 mg. 1.390 mmol) stirring in EtOH (6 mL), was added Pd(OH)₂ (88.5 mg). The stirring mixture was then subjected to an atmosphere of hydrogen for 8 hours at 25° C. The reaction was then diluted with EtOAc and filtered through celite. The filtrate was then concentrated under reduced pressure and the resultant residue then purified by column chromatography (SiO₂, MeOH:DCM—0:1 to 1:9) to afford the title compound (352.5 mg) as a colorless oil.

LCMS: m/z 289.1 [M+H]⁺.

¹H NMR (600 MHz, DMSO) δ 7.24 (dd, J=9.9 Hz, 1H), 7.09 (m, 4H), 6.86 (d, J=8.1 Hz, 0.5H), 6.82 (d, J=8.1 Hz, 0.5H), 4.85 (s, 1H), 4.77 (s, 1H), 4.50 (ds, J=10.6 Hz, 2H), 4.07 (t, J=5.4 Hz, 1H), 4.01 (t, J=5.9 Hz, 1H), 3.74 (d, J=5.6 Hz, 1H), 3.61 (t, J=5.8 Hz, 1H), 3.08 (s, 1.5H), 2.88 (s, 1.5H), 2.23 (ds, J=5.8 Hz, 3H).

### Step 3: N-(1-(2-(methyl(2-(p-tolyloxy)ethyl)amino)-2-oxoethyl)-1H-pyrazol-3-yl) -3-phenoxypropanamide

To a suspension of 3-phenoxypropionic acid (38 mg, 0.217 mmol), HOBt hydrate (40 mg, 0.26 mmol), and EDC HCl (50 mg, 0.260 mmol) stirring under N₂ in DMF (1 mL) at 25° C. was added DIEA (60 μL, 0.347 mmol). The reaction mixture was then allowed to stir briefly before adding a solution of 2-(3-amino-1H-pyrazol-1-yl)-N-methyl-N-(2-(p -tolyloxy)ethyl)acetamide (50 mg. 0.173 mmol) in DMF (1 mL). Stirring was continued at 25° C. for the following 24 hrs. The reaction mixture was then concentrated under reduced pressure and the resultant residue then purified by column chromatography (SiO₂, MeOH:DCM—0:1 to 5:95) to afford the title compound (38.6 mg) as a pale off-white solid.

LCMS: m/z 437.2 [M+H]⁺ and m/z 459.2 [M+Na]⁺

¹H NMR (600 MHz, DMSO-d₆) δ 10.49 (ds, J=9.5 Hz, 1H), 7.53 (dd, J=11.3, 2.4 Hz, 1H), 7.46-7.41 (m, 1H), 7.28 (ddd, J=9.5, 7.2, 2.5 Hz, 2H), 7.09 (dd, J=15.8, 8.3 Hz, 2H), 6.92 (t, J=7.1 Hz, 3H), 6.89-6.86 (d, J=8.43 Hz, 1H), 6.84-6.81 (d, J=8.43 Hz, 1H), 5.09 (s, 1H), 5.00 (s, 1H), 4.23 (t, J=6.1 Hz, 1H), 4.15 (t, J=6.1 Hz, 1H), 4.10 (t, J=5.2 Hz, 1H), 4.03 (t, J=5.8 Hz, 1H), 3.76 (t, J=5.2 Hz, 1H), 3.63 (t, J=5.8 Hz, 1H), 3.11 (s, 1.5H), 2.90 (s, 1.5H), 2.75 (t, J=6.1 Hz, 1H), 2.66 (t, J=6.1 Hz, 1H), 2.23 (ds, J=6.5 Hz, 3H).

| | Synthesized Using | | |
|---|---|---|---|
| | Appropriate | | |
| | Starting Materials | | LCMS |
| Compound | Similar to | | m/z |
| Number | Synthesis of: | ¹H NMR (400 MHz) | [M + H]⁺ |
| 10 | Example 2 | (Acetone-d₆) δ 9.25 (brs, 1H), 7.96 (ds, J = | 467.1 |
| | | 3.3 Hz, 1H), 7.42 (ds, J = 9.8 Hz, 1H), 7.11- | |
| | | 7.00 (m, 4H), 6.97-6.93 (m, 2H), 6.90 (d, | |
| | | J = 8.5 Hz, 1H), 6.83 (d, J = 8.4 Hz, 1H), | |
| | | 5.20 (s, 1H), 5.06 (s, 1H), 4.31 (td, J = 6.1, | |
| | | 2.0 Hz, 2H), 4.19 (t, J = 5.1 Hz, 1H), 4.09 (t, | |
| | | J = 5.7 Hz, 1H), 3.88 (t, J = 5.1 Hz, 1H), | |
| | | 3.72 (t, J = 5.7 Hz, 1H), 3.24 (ds, J = 3.6 | |
| | | Hz, 3H), 2.98 (s, 3H), 2.77 (td, J = 6.2, 1.9 | |
| | | Hz, 2H), 2.24 (ds, J = 3.5 Hz, 3H). | |
| 16 | Example 2 | (Acetone-d₆) δ 9.23 (s, 1H), 7.96 (ds, J = 3.6 | 455.1 |
| | | Hz, 1H), 7.42 (d, J = 10.0 Hz, 1H), 7.09 (dd, | |
| | | J = 10.9, 8.2 Hz, 2H), 6.92-6.80 (m, 6H), | |
| | | 5.20 (s, 1H), 5.06 (s, 1H), 4.26 (td, J = 6.2, | |
| | | 2.1 Hz, 2H), 4.19 (t, J = 5.2 Hz, 1H), 4.09 (t, | |
| | | J = 5.7 Hz, 1H), 3.88 (t, J = 5.1 Hz, 1H), | |
| | | 3.24 (s, 1.5H), 2.98 (s, 1.5H), 2.74 (td, J = | |
| | | 6.2, 2.0 Hz, 2H), 2.24 (ds, J = 3.6 Hz, 3H). | |
| 31 | Example 4 | (CD₃OD) δ = 7.97 (s, 1H), 7.58-7.55 (m, | 489.3 |
| | | 3H), 7.44-7.40 (m, 2H), 7.27-7.25 (d, J = | |
| | | 8.0 Hz, 2H), 7.30-7.25 (m, 2H), 6.93-6.90 | |
| | | (m, 3H), 5.07 & 5.05 (2 singlets, 2H), 4.30 | |
| | | (t, J = 6.0 Hz, 2H), 3.44 (t, J = 7.2 Hz, 2H), | |
| | | 3.08, 2.94 (2 singlets, 3H), 2.80-2.67 (m, | |
| | | 4H), 1.97-1.86 (m, 2H). | |
| 32 | Example 4 | (DMSO-d₆) δ = 10.15, 10.13 (2 singlets, | 504.2 |
| | | 1H), 7.84, 7.83 (2 singlets, 1H), 7.46-7.40 | |
| | | (m, 3H), 7.30-7.25 (m, 2H), 6.95-6.82 | |
| | | (m, 5H), 4.99, 4.95 (2 singlets, 2H), 4.26- | |
| | | 4.23 (m, 2H), 3.87-3.40 (m, 4H), 3.03, | |
| | | 3.00, 2.96 & 2.87 (4 singlets, 6H), 2.73- | |
| | | 2.71 (m, 2H). | |
| 33 | Example 4 | (CD₃OD) δ = 10.11 (s, 1H), 7.90, 7.86 (2 | 491.1 |
| | | singlets, 1H), 7.65-7.45 (m, 1H), 7.43- | |
| | | 7.41 (m, 1H), 7.35-7.25 (m, 5H), 6.96- | |
| | | 6.85 (m, 3H), 5.20, 5.08 (2 singlets, 2H), | |
| | | 4.30-4.10 (m, 4H), 3.85-3.60 (m, 2H), | |
| | | 3.12, 2.72 (2 singlets, 3H), 2.75, 2.65 (m, | |
| | | 2H). | |
| 34 | Example 1 - last | (CD₃OD) δ = 7.96, 7.95 (2 singlets, 1H), | 455.1 |
| | step RCO₂H, | 7.56, 7.55 (2 singlets, 1H), 7.15-7.05 (m, | |
| | EDCI, HOBT, | 5H), 6.92-6.79 (m, 3H), 5.24, 5.11 (2 | |
| | DIEA, DCM | singlets, 2H), 4.38-4.35 (m, 2H), 4.17- | |
| | | 4.08 (m, 2H), 3.85-3.75 (m, 2H), 3.22, 3.02 | |
| | | (2 singlets, 3H), 2.82 (t, J = 6 hz, 2H), 2.26, | |
| | | 2.25 (2 singlets, 3H). | |
| 35 | Example 1 - last | (DMSO-d₆) δ = 7.99, 7.98 (2 singlets, 1H), | 449.1 |
| | step RCO₂H, | 7.61, 7.60 (2 singlets, 1H), 7.25 (d, J = 8.4 | |
| | EDCI, HOBT, | hz, 1H), 7.15-7.00 (m, 2H), 6.95-6.85 | |
| | DIEA, DCM | (m, 3H), 6.80-6.77 (m, 1H), 5.26, 5.13 (2 | |
| | | singlets, 2H), 4.51 (s, 2H), 4.31-4.28 (m, | |
| | | 2H), 4.17-4.05 (m, 2H), 3.83-3.75 (m, | |
| | | 2H), 3.22, 3.02 (2 singlets, 3H), 2.79 (t, J = | |
| | | 6 hz, 2H), 2.26, 2.25 (2 singlets, 3H). | |
| 36 | Example 1 - last | (CD₃OD) δ = 7.96, 7.95 (2 singlets, 1H), | 455.3 |
| | step RCO₂H, | 7.55 (d, J = 3.6 hz, 1H), 7.30-7.20 (m, | |
| | EDCI, HOBT, | 1H), 7.15-7.05 (m, 4H), 7.10-7.00 (m, | |
| | DIEA, DCM | 2H), 6.88 (d, J = 8.4 Hz, 1H), 6.81-6.66 | |
| | | (m, 4H), 5.2, 5.10 (2 singlets, 2H), 4.30 (t, J = | |
| | | 6 hz, 2H), 4.17-4.11 (m, 2H), 3.84-3.74 | |
| | | (m, 2H), 3.22, 3.02 (2 singlets, 3H), 2.79 (t, | |
| | | J = 6 hz, 2H), 2.26, 2.25 (2 singlets, 3H). | |
| 37 | Example 1 - last | (CD₃OD) δ = 7.98-7.95 (bs, 1H), 7.57 (s, | 452.4 |
| | step RCO₂H, | 1H), 7.33-7.28 (m, 2H), 7.15-7.05 (m, | |
| | EDCI, HOBT, | 4H), 6.90-6.85 (m, 1H), 6.85-6.75 (m, | |
| | DIEA, DCM | 1H), 5.25 & 5.11 (2 singlets, 2H), 4.36- | |
| | | 4.33 (m, 2H), 4.20-4.05 (m, 2H), 3.85- | |
| | | 3.75 (m, 2H), 3.22, 3.02 (2 singlets, 3H), | |
| | | 2.825-2.75 (m, 2H), 2.26, 2.25 (2 singlets, | |
| | | 3H) | |
| 38 | Example 4 | (CD₃OD) δ = 8.3-7.95 (m, 3H), 7.59 and | 424.1 |
| | | 7.53 (2 singlets 1H), 7.35-7.15 (m, 4H), | |
| | | 6.95-6.85 (m, 3H), 5.25 & 5.12 (2 singlets, | |
| | | 2H), 4.64-4.50 (m, 2H), 4.35-4.25 (m, | |
| | | 2H), 3.95-3.60 (m, 2H), 3.22, 3.03 (2 | |
| | | singlets, 3H), 2.82-2.75 (m, 2H) | |
| 39 | Example 4 | (CD₃OD) δ = 8.50-8.40 (m, 1H), 8.0- | 492.3 |
| | | 7.79 (m, 2H), 7.53 (d, J = 4.8 Hz, 1H), 7.30- | |
| | | 7.20 (m, 2H), 6.95-6.85 (m, 4H), 5.25 & | |
| | | 5.09 (2 singlets, 2H), 4.65-4.50 (m, 2H), | |
| | | 4.35-4.25 (m, 2H), 3.90-3.75 (m, 2H), | |
| | | 3.31, 3.20 (2 singlets, 3H), 2.80-2.70 (m, | |
| | | 2H) | |
| 41 | Example 2 | (Acetone-d₆) δ 8.40 (ds, 1H), 8.04 (s, 1H), | 423.1 |
| | | 7.50 (s, 1H), 7.37 (ds, 1H) 7.27 (t, J = 7.3 | |
| | | Hz, 2H), 7.06 (d, J = 8.1 Hz, 3H), 6.92 (d, J = | |
| | | 7.4 Hz, 2H), 6.81 (d, J = 8.5 Hz, 2H), 5.11 | |
| | | (s, 1H), 4.80 (s, 1H), 4.29-4.19 (m, 1H), | |
| | | 4.03-3.94 (m, 2H), 3.58 (t, J = 4.1 Hz, | |
| | | 1H), 3.50 (q, J = 5.4 Hz, 2H), 2.23 (s, 3H), | |
| | | 2.21 (m, 1H), 1.89 (m, 1H). | |
| 42 | Example 2 | (Acetone-d₆) δ 9.33 (brs, 1H), 8.05 (s, 1H), | 439.1 |
| | | 7.51 (s, 1H), 7.27 (t, J = 7.8 Hz, 2H), 7.22 | |
| | | (brs, 1H), 6.92 (m, 3H), 6.86-6.82 (m 4H), | |
| | | 4.80 (s, 2H), 4.33 (t, J = 6.2 Hz, 2H), 3.98 (t, | |
| | | J = 5.7 Hz, 2H), 3.73 (s, 3H), 3.56 (q, J = | |
| | | 5.7 Hz, 2H), 2.78 (t, J = 6.2 Hz, 2H). | |
| 43 | Example 2 | (Acetone-d₆) δ 9.30 (s, 1H), 8.00 (ds, J = | 407.1 |
| | | 10.2 Hz, 1H), 7.42 (ds, J = 8.3 Hz, 1H), 7.27 | |
| | | (dd, J = 8.8, 7.3 Hz, 2H), 7.22-7.16 (m, | |
| | | 3H), 7.13 (d, J = 7.8 Hz, 1H), 6.96-6.89 | |
| | | (m, 3H), 5.11 (s, 1H), 5.09 (s, 1H), 4.67 (s, | |
| | | 1H), 4.54 (s, 1H), 4.33 (t, J = 6.2 Hz, 2H), | |
| | | 3.03 (s, 1.5H), 2.87 (s, 1.5H), 2.17 (p, J = | |
| | | 2.2 Hz, 2H), 2.08 (d, J = 1.4 Hz, 3H). | |
| 44 | Example 2 | (Acetone-d₆) δ 9.52 (brs, 1H), 8.02 (ds, J = | 423.1 |
| | | 4.1 Hz, 1H), 7.53 (ds, J = 9.1 Hz, 1H), 7.32 | |
| | | (t, J = 7.7 Hz, 2H), 7.09 (t, J = 11.2, 8.1 Hz, | |
| | | 2H), 7.05-6.97 (m, 3H), 6.90 (d, J = 8.2 | |
| | | Hz, 1H), 6.83 (d, J = 8.2 Hz, 1H), 5.22 (s, | |
| | | 1H), 5.08 (s, 1H), 4.62 (d, J = 2.1 Hz, 2H), | |
| | | 4.19 (t, J = 5.2 Hz, 1H), 4.09 (t, J = 5.6 Hz, | |
| | | 1H), 3.88 (t, J = 5.2 Hz, 1H), 3.72 (t, J = 5.7 | |
| | | Hz, 1H), 2.99 (s, 2H), 2.53 (s, 1H), 2.24 (ds, | |
| | | J = 3.9 Hz, 3H). | |
| 45 | Example 2 | (Acetone-d₆) δ 9.33 (brs, 1H), 7.96 (ds, J = | 471.1 |
| | | 3.7 Hz, 1H), 7.43 (d, J = 9.6 Hz, 1H), 7.28 | |
| | | (d, J = 8.4 Hz, 2H), 7.08 (t, J = 9.4 Hz, 2H), | |
| | | 6.96 (d, J = 8.5 Hz, 2H), 6.90 (d, J = 8.2 Hz, | |
| | | 1H), 6.83 (d, J = 8.1 Hz, 1H), 5.20 (s, 1H), | |
| | | 5.06 (s, 1H), 4.32 (t, J = 5.5 Hz, 2H), 4.19 (t, | |
| | | J = 5.1 Hz, 1H), 4.08 (t, J = 5.6 Hz, 1H), | |
| | | 3.87 (t, J = 5.1 Hz, 1H), 3.72 (t, J = 5.7 Hz, | |
| | | 1H), 3.23 (s, 1.5H), 2.98 (s, 1.5H), 2.78 (t, J = | |
| | | 5.8 Hz, 2H), 2.24 (d, J = 3.5 Hz, 3H). | |
| 46 | Example 2 | (Acetone-d₆) 1H NMR (500 MHz, Acetone) | 451.2 |
| | | δ 9.29 (brs, 1H), 8.02 (ds, J = 8.1 Hz, 1H), | |
| | | 7.43 (ds, J = 14.2 Hz, 1H), 7.30-7.24 (m, | |
| | | 2H), 7.11-7.04 (m, 2H), 6.96-6.90 (m, | |
| | | 3H), 6.83 (dd, J = 15.6, 8.4 Hz, 2H), 5.73- | |
| | | 5.67 (m, 0.5H), 5.50-5.468 (m, 0.5H) 5.35 | |
| | | (t, J = 5.1 Hz, 2H), 4.32 (t, J = 6.1 Hz, 2H), | |
| | | 4.07 (m, 2H), 3.15 (s, 1.5H), 2.98 (s, 1.5H), | |
| | | 2.23 (ds, J = 3.9 Hz, 3H), 2.17 (p, J = 2.1 | |
| | | Hz, 2H), 2.08 (ds, J = 1.4 Hz, 3H). | |
| 47 | Example 2 | (Acetone-d₆) δ 8.31 (s, 1H), 7.32-7.22 (m, | 465.2 |
| | | 2H), 7.09 (t, J = 9.2 Hz, 2H), 6.94 (m, 4H), | |
| | | 6.83 (d, J = 8.3 Hz, 1H), 5.09 (s, 1H), 4.94 | |
| | | (s, 1H), 4.34 (td, J = 6.1, 3.0 Hz, 2H), 4.18 | |
| | | (t, J = 5.2 Hz, 1H), 4.09 (t, J = 5.7 Hz, 1H), | |
| | | 3.90 (t, J = 5.2 Hz, 1H), 3.73 (t, J = 5.8 Hz, | |
| | | 1H), 3.25 (s, 1.5H), 2.98 (s, 1.5H), 2.81 (td, | |
| | | J = 6.2, 3.1 Hz, 2H), 2.24 (ds, J = 2.2 Hz, | |
| | | 3H). | |
| 49 | Example 2 | (Acetone-d₆) δ 9.16 (s, 1H), 8.46 (s, 1H), | 423.1 |
| | | 7.98 (brs, 0.5H), 7.75 (brs, 0.5H), 7.42 (brs, | |
| | | 1H), 7.16 (d, J = 7.4 Hz, 1H), 7.13-7.04 | |
| | | (m, 3H), 6.89 (dd, J = 8.4, 5.9 Hz, 1H), 6.82 | |
| | | (m, 2H), 6.77 (d, J = 7.7 Hz, 1H), 5.18 (ds, J = | |
| | | 5.4 Hz, 1H), 5.04 (ds, J = 5.7 Hz, 1H), | |
| | | 4.19 (q, J = 5.6 Hz, 1H), 4.08 (q, J = 5.9 Hz, | |
| | | 1H), 3.87 (q, J = 5.7 Hz, 1H), 3.72 (q, J = | |
| | | 6.0 Hz, 1H), 3.67 (s, 1H), 3.52 (s, 1H), 3.23 | |
| | | (ds, J = 5.6 Hz, 1.5H), 2.98 (ds, J = 7.0 Hz, | |
| | | 1.5H), 2.24 (ds, J = 3.8 Hz, 3H). | |
| 51 | Example 9 | (600 MHz, DMSO-d₆) δ 10.47 (d, J = 9.3 Hz, | 467.1 |
| | | 1H), 7.52 (ds, J = 11.2 Hz, 1H), 7.09 (dd, J = | |
| | | 15.6, 8.1 Hz, 2H), 6.89-6.81 (m, 6H), 6.49 | |
| | | (dd, J = 4.5, 2.3 Hz, 1H), 5.08 (s, 1H), 5.00 | |
| | | (s, 1H), 4.16 (td, J = 6.1, 2.2 Hz, 2H), 4.12- | |
| | | 4.08 (m, 1H), 4.02 (t, J = 5.8 Hz, 1H), 3.76 | |
| | | (t, J = 5.1 Hz, 1H), 3.69 (s, 3H), 3.63 (t, J = | |
| | | 5.7 Hz, 1H), 3.11 (s, 1.5H), 2.90 (s, 1.5H), | |
| | | 2.72 (td, J = 6.1, 3.5 Hz, 2H), 2.23 (ds, J = | |
| | | 6.4 Hz, 3H). | |
| 52 | Example 9 | | 455.1 |
| 53 | Example 1 - last | (CD₃OD) δ = 7.97, 7.96 (2 singlets, 1H), | 435.2 |
| | step RCO₂H, | 7.55 7.54 (2 singlets, 1H), 7.30-7.15 (m, | |
| | EDCI, HOBT, | 4H), 7.15-7.00 (m, 1H), 6.95-6.85 (m, | |
| | DIEA, DCM | 3H), 5.24 5.09 (2 singlets, 2H), 4.35-4.25 | |
| | | (m, 2H), 4.25-4.10 (m, 2H), 3.90-3.70 | |
| | | (m, 2H), 3.31, 3.00 (2 singlets, 3H), 2.85- | |
| | | 2.73 (m, 2H) | |

### Example 10

IRE1-XBP1s activation Assay: HEK293 T-REx cells stably expressing the XBP1-RLuc splicing reporter were treated with an IRE1—XBP1s activator provided herein (10 μM) in the presence or absence of the IRE1 active site inhibitor 4 μ8C (32 μM) for 18 h. Luminescence was shown as the percentage signal relative to thapsigargin (Tg) (500 nM, 18 h). Results are shown in Table 2 below.

TABLE 2
IRE1-XBP1s Activation Data

| Compound # | Activity* |
|---|---|
| 10 | ++ |
| 16 | ++ |
| 25 | + |
| 26 | +/− |
| 27 | +/− |
| 28 | +/− |
| 29 | +/− |
| 30 | ++ |
| 31 | +/− |
| 32 | +/− |
| 33 | + |
| 34 | ++ |
| 35 | ++ |
| 36 | ++ |
| 37 | ++ |
| 38 | +/− |
| 39 | + |
| 40 | +/− |
| 41 | +/− |
| 42 | +/− |
| 43 | +/− |
| 44 | + |
| 45 | + |
| 46 | + |
| 47 | +/− |
| 50 | +/− |
| 51 | +/− |
| 53 | + |

*++: % Tg >= 50%, +: 30% =< % Tg < 50%; +/−: % Tg % < 30%

This disclosure is not to be limited in scope by the embodiments disclosed in the examples which are intended as single illustrations of individual aspects, and any equivalents are within the scope of this disclosure. Various modifications in addition to those shown and described herein will become apparent to those skilled in the art from the foregoing description. Such modifications are intended to fall within the scope of the appended claims.

Various references such as patents, patent applications, and publications are cited herein, the disclosures of which are hereby incorporated by reference herein in their entireties.

What is claimed is:

1. A compound having Formula I: 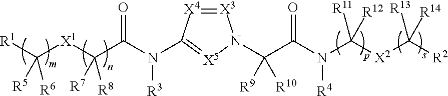
or a pharmaceutically acceptable derivative thereof, wherein:
m, n, p and s are each independently an integer from 0-4;
R¹ and R² are each independently aryl or heteroaryl;
X¹ and X² are each independently a bond, O or NR¹⁵;
X³ and X⁴ are each independently CR¹⁶ or N;
X⁵ is CR¹⁷ or N; and
R³ to R¹⁷ are selected from (i) to (ix):
(i) R³, R⁴ and R¹⁵ are each independently H, alkyl, aryl or aralkyl, and R⁵ to R¹⁴, R¹⁶ and R¹⁷ are each independently H, halo, alkyl, aryl or aralkyl;
(ii) R³ and R⁷, together with the atoms to which they are attached, form a 5-8 membered heterocyclic ring, R⁴ and R¹⁵ are each independently H, alkyl, aryl or aralkyl, and R⁵, R⁶, R⁸ to R¹⁴, R¹⁶ and R¹⁷ are each independently H, halo, alkyl, aryl or aralkyl;
(iii) R⁴ and R⁹, together with the atoms to which they are attached, form a 5-8 membered heterocyclic ring, R³ and R¹⁵ are each independently H, alkyl, aryl or aralkyl, and R⁵ to R⁸, R¹⁰ to R¹⁴, R¹⁶ and R¹⁷ are each independently H, halo, alkyl, aryl or aralkyl;
(iv) R³ and R⁷, together with the atoms to which they are attached, form a 5-8 membered heterocyclic ring, R⁴ and R⁹, together with the atoms to which they are attached, form a 5-8 membered heterocyclic ring, R¹⁵ is independently H, alkyl, aryl or aralkyl, and R⁵, R⁶, R⁸, R¹⁰ to R¹⁴, R¹⁶ and R¹⁷ are each independently H, halo, alkyl, aryl or aralkyl;
(v) R⁴ and R¹¹, together with the atoms to which they are attached, form a 3-8 membered heterocyclic ring, R³ and R¹⁵ are each independently H, alkyl, aryl or aralkyl, and R⁵ to R¹⁰, R¹² to R¹⁴, R¹⁶ and R¹⁷ are each independently H, halo, alkyl, aryl or aralkyl;
(vi) R¹¹ and R¹², together with the atoms to which they are attached, form a 3-8 membered heterocyclic ring, R³, R⁴ and R¹⁵ are each independently H, alkyl, aryl or aralkyl, and R⁵ to R¹⁰, R¹³, R¹⁴, R¹⁶ and R¹⁷ are each independently H, halo, alkyl, aryl or aralkyl;
(vii) when X² is NR¹⁵, R¹¹ and R¹⁵, together with the atoms to which they are attached, form a 3-8 membered heterocyclic ring, R³ and R⁴ are each independently H, alkyl, aryl or aralkyl, and R⁵ to R¹⁰, R¹² to R¹⁴, R¹⁶ and R¹⁷ are each independently H, halo, alkyl, aryl or aralkyl;
(viii) R⁷ and R⁸, together with the atoms to which they are attached, form a 3-8 membered heterocyclic ring, R³, R⁴ and R¹⁵ are each independently H, alkyl, aryl or aralkyl, and R⁵, R⁶, R⁹ to R¹⁴, R¹⁶ and R¹⁷ are each independently H, halo, alkyl, aryl or aralkyl; or
(ix) when X¹ is NR¹⁵, R⁷ and R¹⁵, together with the atoms to which they are attached, form a 3-8 membered heterocyclic ring, R³ and R⁴ are each independently H, alkyl, aryl or aralkyl, and R⁵, R⁶, R⁸ to R¹⁴, R¹⁶ and R¹⁷ are each independently H, halo, alkyl, aryl or aralkyl;
with the proviso that when X³ is N, X⁴ is CR¹⁶ and X⁵ is CR¹⁷; then R¹⁶ and R¹⁷ are not both methyl or trifluoromethyl; and
with the proviso that R⁹ and R¹⁰ are not both methyl; and
with the proviso that the compound is not N-(1-(2-(methyl(2-(p-tolyloxy)ethyl)amino)-2-oxoethyl)-1H-pyrazol-4-yl)-3-phenoxypropanamide.

2. The compound of claim 1, or a pharmaceutically acceptable derivative thereof, wherein:
m, n, p and s are each independently an integer from 0-4;
R¹ and R² are each independently aryl or heteroaryl;
X¹ and X² are each independently a bond, O or NR¹⁵;
X³ and X⁴ are each independently CR¹⁶ or N;
X⁵ is CR¹⁷ or N;
R³, R⁴ and R¹⁵ are each independently H, alkyl, aryl or aralkyl; and
R⁵ to R¹⁴, R¹⁶ and R¹⁷ are each independently H, halo, alkyl, aryl or aralkyl.

3. The compound of claim 1 or claim 2, or a pharmaceutically acceptable derivative thereof, wherein:
R¹ and R² are each independently aryl or heteroaryl;
X¹ and X² are each independently a bond or O;
X³ and X⁴ are each independently CH or N;
X⁵ is CH; and
R³ to R¹⁵ are each independently H or alkyl.

4. The compound of any one of claims 1-3, or a pharmaceutically acceptable derivative thereof, wherein:
R¹ and R² are each independently aryl or heteroaryl;
X¹ and X² are each independently a bond or O;
X³ and X⁴ are each independently CH or N;
X⁵ is CH; and
R³ to R¹⁵ are each independently H or methyl.

5. The compound of claim 1 or claim 2, wherein:
R¹ is aryl;
R² is aryl or heteroaryl;
X¹ is a bond or O;
X² is a bond, O or NR¹⁵;
X³ is CH or N;
X⁴ is CR¹⁶;
X⁵ is CR¹⁷ or N;
R³ to R¹⁷ are each independently H or methyl;
m and s are 0;
n is 0, 1, 2 or 3; and
p is 0 1, 2 or 3.

6. The compound of any one of claims 1, 2 and 5, wherein:
R¹ is aryl;
R² is aryl or heteroaryl;
X¹ is a bond or O;
X² is a bond, O or NCH₃;
X³ is CH or N;
X⁴ is CH or CCH₃;
X⁵ is CH, CCH₃ or N;
R⁴ and R⁹ are each independently H or methyl;
R³, R⁵ to R⁸, and R¹⁰ to R¹⁶ are each H;
m and s are 0;
n is 0, 1, 2 or 3; and
p is 0 1, 2 or 3.

7. The compound of any one of claims 1-4, wherein R¹ is phenyl or pyridyl.

8. The compound of any one of claims 1-4 and 7, wherein R¹ is phenyl or pyridyl, each optionally substituted with halo or haloalkyl.

9. The compound of any one of claims 1-4, 7 and 8, wherein R¹ is phenyl or pyridyl, each optionally substituted with fluoro or trifluoromethyl.

10. The compound of any one of claims 1-4 and 7-9, wherein R¹ is 4-fluorophenyl, 4-trifluoromethylphenyl or 2-pyridyl.

11. The compound of any one of claims 1-7, wherein R¹ is phenyl optionally substituted with halo, alkyl, amino, hydroxy, hydroxyalkyl or alkoxy.

12. The compound of any one of claims 1-7 and 11, wherein R¹ is phenyl optionally substituted with fluoro, chloro, hydroxymethyl, amino, hydroxy or methoxy.

13. The compound of any one of claims 1-7, 11 and 12, wherein R¹ is phenyl, 2-fluorophenyl, 3-fluorophenyl, 4-fluorophenyl, 4-hydroxymethylphenyl, 4-aminophenyl, 4-chlorophenyl, 4-hydroxyphenyl or 4-methoxyphenyl.

14. The compound of any one of claims 1-13, wherein R² is phenyl or pyridyl.

15. The compound of any one of claims 1-14, wherein R² is phenyl or pyridyl, each optionally substituted with alkyl, alkoxy, halo or haloalkyl.

16. The compound of any one of claims 1-15, wherein R² is phenyl or pyridyl, each optionally substituted with methyl, methoxy, fluoro or trifluoromethyl.

17. The compound of any one of claims 1-16, wherein R² is 4-methylphenyl, 4-methoxyphenyl, 4-fluorophenyl, 4-trifluoromethylphenyl or 2-pyridyl.

18. The compound of any one of claims 1-14, wherein R² is phenyl or pyridyl, each optionally substituted with alkyl, alkoxy, diazirinyl or haloalkyl.

19. The compound of any one of claims 1-14 and 18, wherein R² is phenyl or pyridyl, each optionally substituted with methyl, methoxy, 3-(trifluoromethyl)-3H-diazirin-3-yl or trifluoromethyl.

20. The compound of any one of claims 1-14, 18 and 19, wherein R² is phenyl, 4-methylphenyl, 4-trifluoromethylphenyl, 2-pyridyl, 5-trifluoromethyl-2-pyridyl, 4-methoxyphenyl or 3-(trifluoromethyl)-3H-diazirin-3-yl.

21. The compound of any one of claims 1-20, wherein X¹ is a bond or O.

22. The compound of any one of claims 1-21, wherein X¹ is a bond.

23. The compound of any one of claims 1-21, wherein X¹ is O.

24. The compound of any one of claims 1-23, wherein X² is a bond or O.

25. The compound of any one of claims 1-24, wherein X² is a bond.

26. The compound of any one of claims 1-24, wherein X² is O.

27. The compound of any one of claims 1, 2 and 5-23, wherein X² is a bond, O or NCH₃.

28. The compound of any one of claims 1, 2, 5-23 and 27, wherein X² is NCH₃.

29. The compound of any one of claims 1-4 and 7-28, wherein X³ is CH and X⁴ is N.

30. The compound of any one of claims 1-28, wherein X³ is N and X⁴ is CH.

31. The compound of any one of claims 1, 2 and 7-28, wherein X³ is N or CH.

32. The compound of any one of claims 1, 2, 5-28 and 31, wherein X⁴ is CH or CCH₃.

33. The compound of any one of claims 1, 2, 5 and 7-32, wherein X⁵ is CR¹⁷.

34. The compound of any one of claims 1, 2 and 5-33, wherein X⁵ is CH.

35. The compound of any one of claims 1, 2 and 5-32, wherein X⁵ is N.

36. The compound of any one of claims 1, 2 and 5-33, wherein X⁵ is CCH₃.

37. The compound of any one of claims 1-4 and 7-29, wherein X³ is CH, X⁴ is N and X⁵ is CH.

38. The compound of any one of claims 1-4 and 7-28, wherein X³ is N, X⁴ is CH and X⁵ is CH.

39. The compound of any one of claims 1, 2 and 5-28, wherein X³ is N, X⁴ is CCH₃ and X⁵ is CCH₃.

40. The compound of any one of claims 1, 2 and 5-28, wherein X³ is CH, X⁴ is CH and X⁵ is N.

41. The compound of any one of claims 1-40, wherein R³, R⁴ and R¹⁵ are each independently H, alkyl, aryl or aralkyl, and R⁵ to R¹⁴, R¹⁶ and R¹⁷ are each independently H, halo, alkyl, aryl or aralkyl.

42. The compound of any one of claims 1-41, wherein R³, R⁴ and R¹⁵ are each independently H or alkyl.

43. The compound of any one of claims 1-42, wherein R³, R⁴ and R¹⁵ are each independently H or methyl.

44. The compound of any one of claims 1-43, wherein R⁵ to R¹⁴, R¹⁶ and R¹⁷ are each independently H or alkyl.

45. The compound of any one of claims 1-44, wherein R⁵ to R¹⁴, R¹⁶ and R¹⁷ are each independently H or methyl.

46. The compound of any one of claims 1-45, wherein R⁵ to R¹⁴, R¹⁶ and R¹⁷ are each independently H.

47. The compound of any one of claims 1-45, wherein R³, R⁴, R⁹, R¹⁶ and R¹⁷ are H or methyl, R⁵ to R⁸ and R¹⁰ to R¹⁴ are H, and R¹⁵ is methyl.

48. The compound of any one of claims 1-41, wherein R³ is H or alkyl.

49. The compound of any one of claims 1-42, wherein R³ is H or methyl.

50. The compound of any one of claims 1-42, wherein R³ is H.

51. The compound of any one of claims 1-42, wherein R³ is methyl.

52. The compound of any one of claims 1-41, wherein R⁴ is H or alkyl.

53. The compound of any one of claims 1-42, wherein R⁴ is H or methyl.

54. The compound of any one of claims 1-42, wherein R⁴ is H.

55. The compound of any one of claims 1-42, wherein R⁴ is methyl.

56. The compound of any one of claims 1-43, wherein R⁷ is H or alkyl.

57. The compound of any one of claims 1-43, wherein R⁷ is H or methyl.

58. The compound of any one of claims 1-43, wherein R⁷ is H.

59. The compound of any one of claims 1-43, wherein R⁷ is methyl.

60. The compound of any one of claim 1, 2 or 7-43, wherein R⁹ is H or alkyl.

61. The compound of any one of claim 1, 2 or 7-43, wherein R⁹ is H or methyl.

62. The compound of any one of claim 1, 2 or 7-43, wherein R⁹ is H.

63. The compound of any one of claim 1, 2 or 7-43, wherein R⁹ is methyl.

64. The compound of any one of claims 1-4 and 7-63, wherein m is 0.

65. The compound of any one of claims 1-5 and 7-64, wherein n is 0, 1, 2 or 3.

66. The compound of any one of claims 1-65, wherein n is 2.

67. The compound of any one of claims 1-4 and 7-66, wherein p is 0, 1, 2 or 3.

68. The compound of any one of claims 1-67, wherein p is 2.

69. The compound of any one of claims 1-4 and 7-68, wherein s is 0.

70. The compound of any one of claims 1-69, wherein the compound is selected from those shown in Table 1 below:

| Compound | Structure |
|---|---|
| 1 | 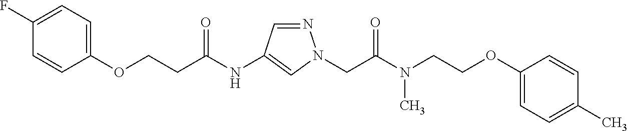 [SMILES: Cc1ccc(OCCN(C)C(=O)Cn2cc(NC(=O)CCOc3ccc(F)cc3)cn2)cc1] |
| 2 | 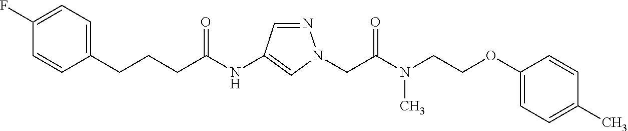 [SMILES: Cc1ccc(OCCN(C)C(=O)Cn2cc(NC(=O)CCCc3ccc(F)cc3)cn2)cc1] |
| 3 | 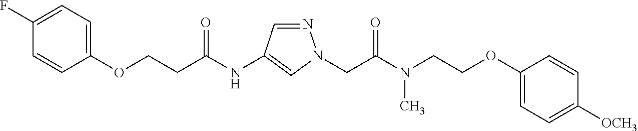 [SMILES: COc1ccc(OCCN(C)C(=O)Cn2cc(NC(=O)CCOc3ccc(F)cc3)cn2)cc1] |
| 4 | 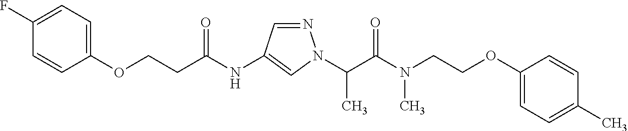 [SMILES: Cc1ccc(OCCN(C)C(=O)C(C)n2cc(NC(=O)CCOc3ccc(F)cc3)cn2)cc1] |
| 5 | 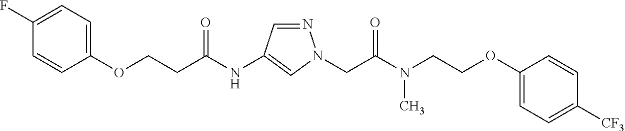 [SMILES: CN(CCOc1ccc(C(F)(F)F)cc1)C(=O)Cn1cc(NC(=O)CCOc2ccc(F)cc2)cn1] |
| 6 | 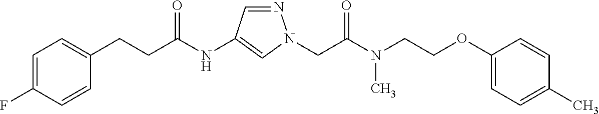 [SMILES: Cc1ccc(OCCN(C)C(=O)Cn2cc(NC(=O)CCc3ccc(F)cc3)cn2)cc1] |
| 7 | 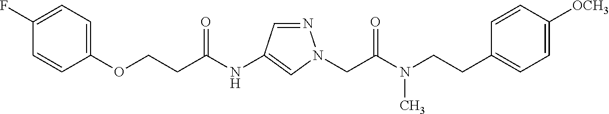 [SMILES: COc1ccc(CCN(C)C(=O)Cn2cc(NC(=O)CCOc3ccc(F)cc3)cn2)cc1] |
| 8 | 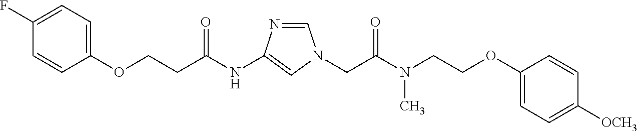 [SMILES: COc1ccc(OCCN(C)C(=O)Cn2cnc(NC(=O)CCOc3ccc(F)cc3)c2)cc1] |
| 9 | 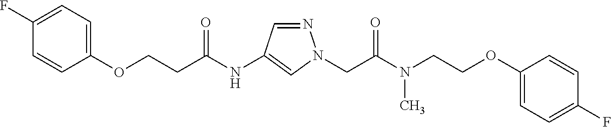 [SMILES: CN(CCOc1ccc(F)cc1)C(=O)Cn1cc(NC(=O)CCOc2ccc(F)cc2)cn1] |
| 10 | 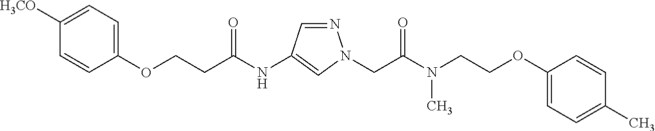 [SMILES: COc1ccc(OCCC(=O)Nc2cnn(CC(=O)N(C)CCOc3ccc(C)cc3)c2)cc1] |
| 11 | 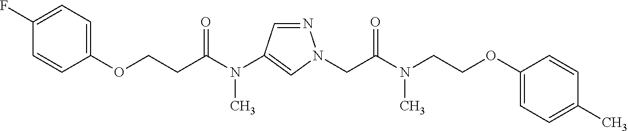 [SMILES: Cc1ccc(OCCN(C)C(=O)Cn2cc(N(C)C(=O)CCOc3ccc(F)cc3)cn2)cc1] |
| 12 | 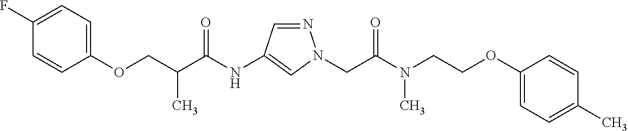 [SMILES: Cc1ccc(OCCN(C)C(=O)Cn2cc(NC(=O)C(C)COc3ccc(F)cc3)cn2)cc1] |
| 13 | 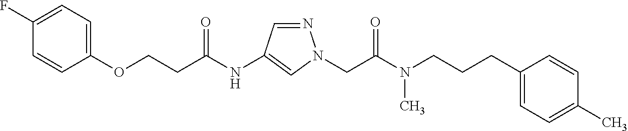 [SMILES: Cc1ccc(CCCN(C)C(=O)Cn2cc(NC(=O)CCOc3ccc(F)cc3)cn2)cc1] |
| 14 | 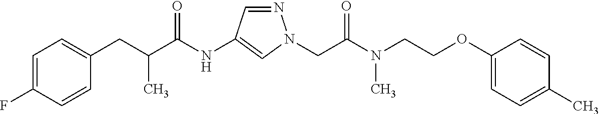 [SMILES: Cc1ccc(OCCN(C)C(=O)Cn2cc(NC(=O)C(C)Cc3ccc(F)cc3)cn2)cc1] |
| 15 | 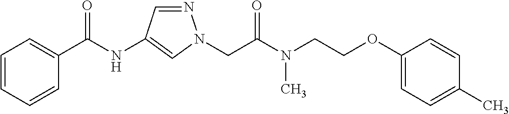 [SMILES: Cc1ccc(OCCN(C)C(=O)Cn2cc(NC(=O)c3ccccc3)cn2)cc1] |
| 16 | 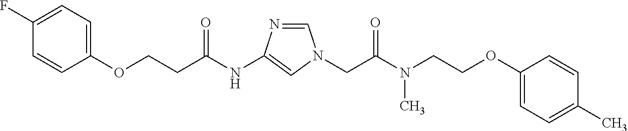 [SMILES: Cc1ccc(OCCN(C)C(=O)Cn2cnc(NC(=O)CCOc3ccc(F)cc3)c2)cc1] |
| 17 | 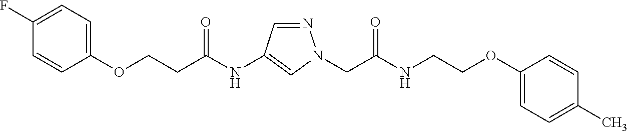 [SMILES: Cc1ccc(OCCNC(=O)Cn2cc(NC(=O)CCOc3ccc(F)cc3)cn2)cc1] |
| 18 | 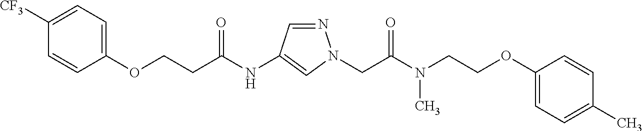 [SMILES: Cc1ccc(OCCN(C)C(=O)Cn2cc(NC(=O)CCOc3ccc(C(F)(F)F)cc3)cn2)cc1] |
| 19 | 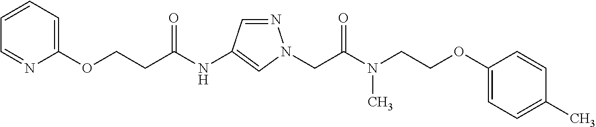 [SMILES: Cc1ccc(OCCN(C)C(=O)Cn2cc(NC(=O)CCOc3ccccn3)cn2)cc1] |
| 20 | 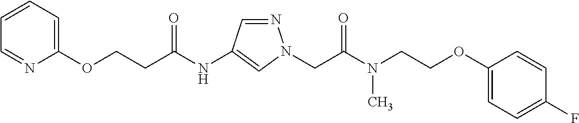 [SMILES: CN(CCOc1ccc(F)cc1)C(=O)Cn1cc(NC(=O)CCOc2ccccn2)cn1] |
| 21 | 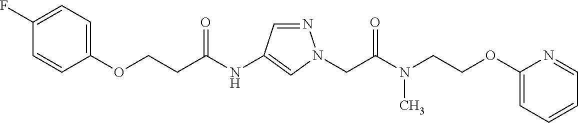 [SMILES: CN(CCOc1ccccn1)C(=O)Cn1cc(NC(=O)CCOc2ccc(F)cc2)cn1] |
| 22 | 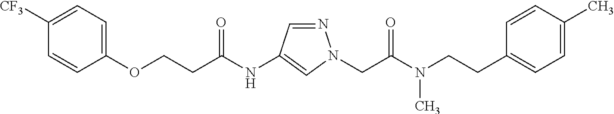 [SMILES: Cc1ccc(CCN(C)C(=O)Cn2cc(NC(=O)CCOc3ccc(C(F)(F)F)cc3)cn2)cc1] |
| 23 | 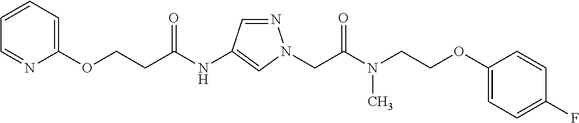 [SMILES: CN(CCOc1ccc(F)cc1)C(=O)Cn1cc(NC(=O)CCOc2ccccn2)cn1] |
| 24 | 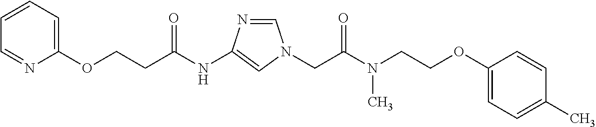 [SMILES: Cc1ccc(OCCN(C)C(=O)Cn2cnc(NC(=O)CCOc3ccccn3)c2)cc1] |
| 25 | 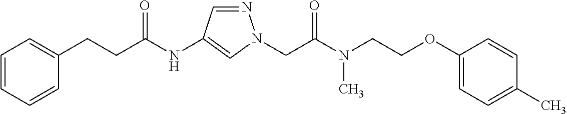 [SMILES: Cc1ccc(OCCN(C)C(=O)Cn2cc(NC(=O)CCc3ccccc3)cn2)cc1] |
| 26 | 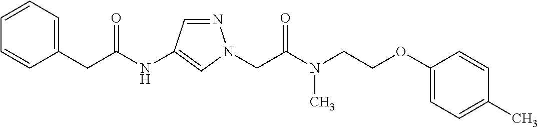 [SMILES: Cc1ccc(OCCN(C)C(=O)Cn2cc(NC(=O)Cc3ccccc3)cn2)cc1] |
| 27 | 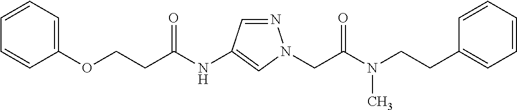 [SMILES: CN(CCc1ccccc1)C(=O)Cn1cc(NC(=O)CCOc2ccccc2)cn1] |
| 28 | 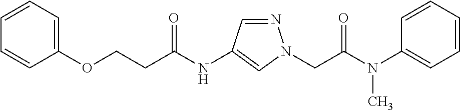 [SMILES: CN(C(=O)Cn1cc(NC(=O)CCOc2ccccc2)cn1)c1ccccc1] |
| 29 | 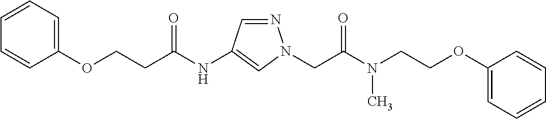 [SMILES: CN(CCOc1ccccc1)C(=O)Cn1cc(NC(=O)CCOc2ccccc2)cn1] |
| 30 | 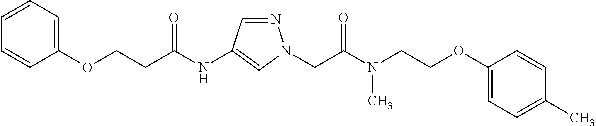 [SMILES: Cc1ccc(OCCN(C)C(=O)Cn2cc(NC(=O)CCOc3ccccc3)cn2)cc1] |
| 31 | 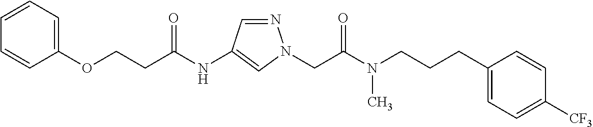 [SMILES: CN(CCCc1ccc(C(F)(F)F)cc1)C(=O)Cn1cc(NC(=O)CCOc2ccccc2)cn1] |
| 32 | 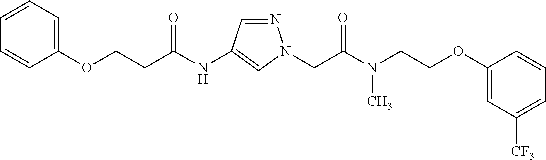 [SMILES: CN(CCOc1cccc(C(F)(F)F)c1)C(=O)Cn1cc(NC(=O)CCOc2ccccc2)cn1] |
| 33 | 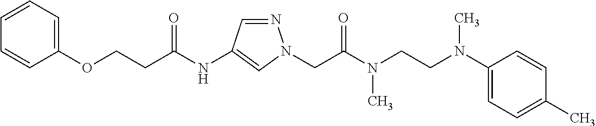 [SMILES: Cc1ccc(N(C)CCN(C)C(=O)Cn2cc(NC(=O)CCOc3ccccc3)cn2)cc1] |
| 34 | 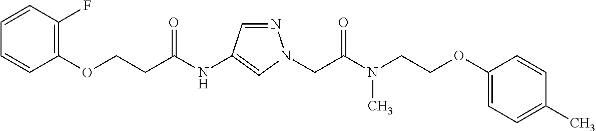 [SMILES: Cc1ccc(OCCN(C)C(=O)Cn2cc(NC(=O)CCOc3ccccc3F)cn2)cc1] |
| 35 | 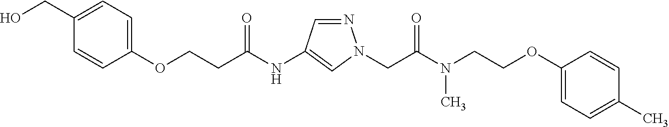 [SMILES: Cc1ccc(OCCN(C)C(=O)Cn2cc(NC(=O)CCOc3ccc(CO)cc3)cn2)cc1] |
| 36 | 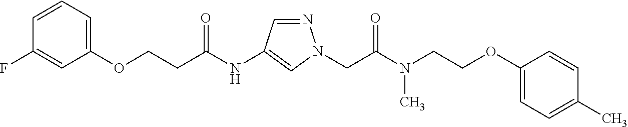 [SMILES: Cc1ccc(OCCN(C)C(=O)Cn2cc(NC(=O)CCOc3cccc(F)c3)cn2)cc1] |
| 37 | 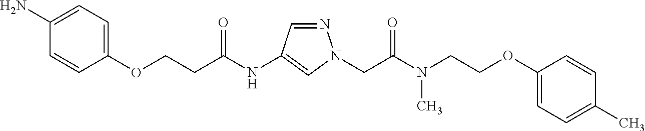 [SMILES: Cc1ccc(OCCN(C)C(=O)Cn2cc(NC(=O)CCOc3ccc(N)cc3)cn2)cc1] |
| 38 | 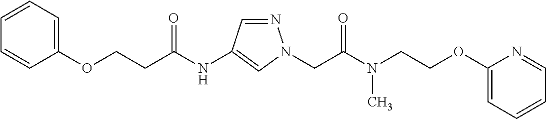 [SMILES: CN(CCOc1ccccn1)C(=O)Cn1cc(NC(=O)CCOc2ccccc2)cn1] |
| 39 | 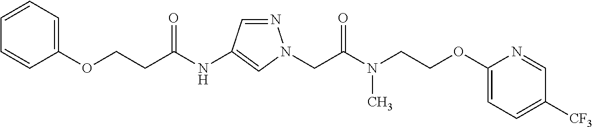 [SMILES: CN(CCOc1ccc(C(F)(F)F)cn1)C(=O)Cn1cc(NC(=O)CCOc2ccccc2)cn1] |
| 40 | 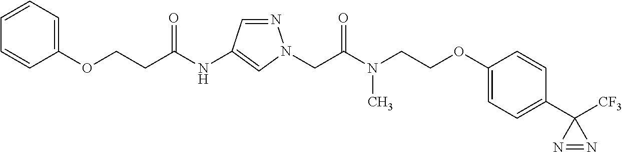 [SMILES: CN(CCOc1ccc(C2(C(F)(F)F)N=N2)cc1)C(=O)Cn1cc(NC(=O)CCOc2ccccc2)cn1] |
| 41 | 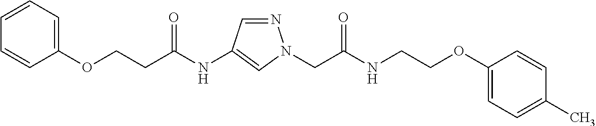 [SMILES: Cc1ccc(OCCNC(=O)Cn2cc(NC(=O)CCOc3ccccc3)cn2)cc1] |
| 42 | 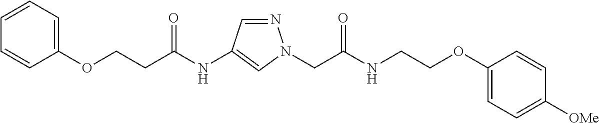 [SMILES: COc1ccc(OCCNC(=O)Cn2cc(NC(=O)CCOc3ccccc3)cn2)cc1] |
| 43 | 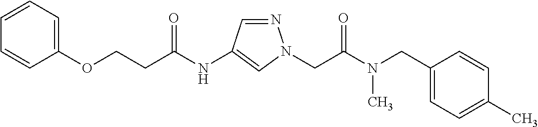 [SMILES: Cc1ccc(CN(C)C(=O)Cn2cc(NC(=O)CCOc3ccccc3)cn2)cc1] |
| 44 | 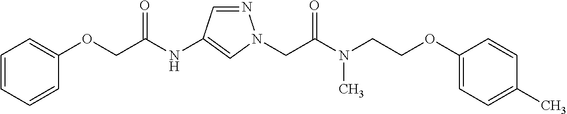 [SMILES: Cc1ccc(OCCN(C)C(=O)Cn2cc(NC(=O)COc3ccccc3)cn2)cc1] |
| 45 | 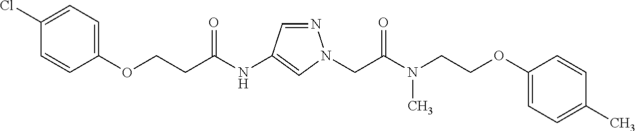 [SMILES: Cc1ccc(OCCN(C)C(=O)Cn2cc(NC(=O)CCOc3ccc(Cl)cc3)cn2)cc1] |
| 46 | 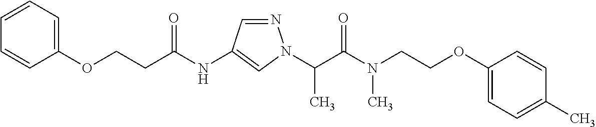 [SMILES: Cc1ccc(OCCN(C)C(=O)C(C)n2cc(NC(=O)CCOc3ccccc3)cn2)cc1] |
| 47 | 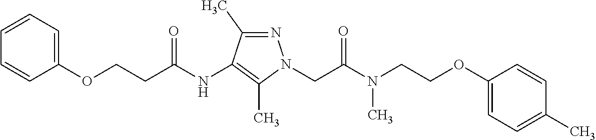 [SMILES: Cc1ccc(OCCN(C)C(=O)Cn2nc(C)c(NC(=O)CCOc3ccccc3)c2C)cc1] |
| 48 | 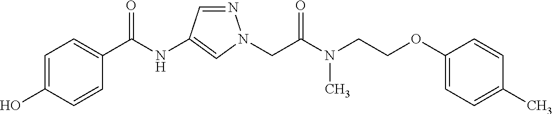 [SMILES: Cc1ccc(OCCN(C)C(=O)Cn2cc(NC(=O)c3ccc(O)cc3)cn2)cc1] |
| 49 | 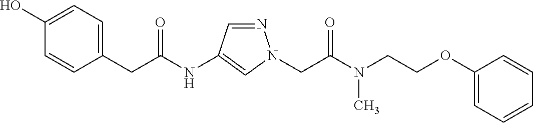 [SMILES: CN(CCOc1ccccc1)C(=O)Cn1cc(NC(=O)Cc2ccc(O)cc2)cn1] |
| 50 | 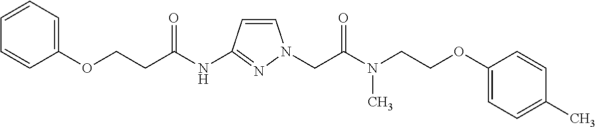 [SMILES: Cc1ccc(OCCN(C)C(=O)Cn2ccc(NC(=O)CCOc3ccccc3)n2)cc1] |
| 51 | 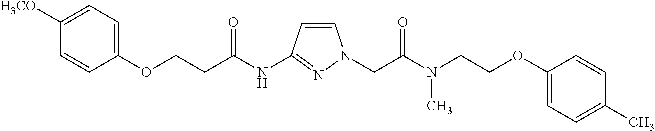 [SMILES: COc1ccc(OCCC(=O)Nc2ccn(CC(=O)N(C)CCOc3ccc(C)cc3)n2)cc1] |
| 52 | 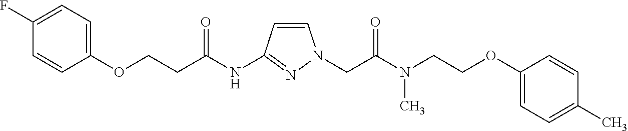 [SMILES: Cc1ccc(OCCN(C)C(=O)Cn2ccc(NC(=O)CCOc3ccc(F)cc3)n2)cc1] |
| 53 | 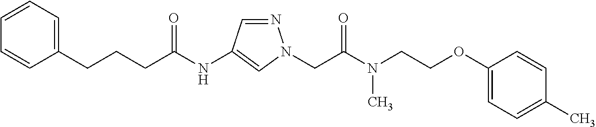 [SMILES: Cc1ccc(OCCN(C)C(=O)Cn2cc(NC(=O)CCCc3ccccc3)cn2)cc1] |

71. A pharmaceutical composition, comprising a compound of any one of claims 1-70 and a pharmaceutically acceptable carrier.

72. A method of treating a disease or disorder selected from a cardiovascular disease, neurodegenerative disease, metabolic disorder, hepatic disorder, protein misfolding disorder or gastrointestinal disorder in a subject, comprising administering to the subject the compound of any one of claims 1-70 or the pharmaceutical composition of claim 71.

73. A method of treating a disease or disorder selected from a cardiovascular disease, neurodegenerative disease, metabolic disorder, hepatic disorder, protein misfolding disorder or gastrointestinal disorder in a subject, comprising administering to the subject the compound of Formula I: 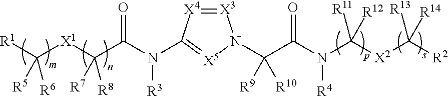
or a pharmaceutically acceptable derivative thereof, wherein:
m, n, p and s are each independently an integer from 0-4;
R¹ and R² are each independently aryl or heteroaryl;
X¹ and X² are each independently a bond, O or NR¹⁵;
X³ and X⁴ are each independently CR¹⁶ or N;
X⁵ is CR¹⁷ or N; and
R³ to R¹⁷ are selected from (i) to (ix):
(i) R³, R⁴ and R¹⁵ are each independently H, alkyl, aryl or aralkyl, and R⁵ to R¹⁴, R¹⁶ and R¹⁷ are each independently H, halo, alkyl, aryl or aralkyl;
(ii) R³ and R⁷, together with the atoms to which they are attached, form a 5-8 membered heterocyclic ring, R⁴ and R¹⁵ are each independently H, alkyl, aryl or aralkyl, and R⁵, R⁶, R⁸ to R¹⁴, R¹⁶ and R¹⁷ are each independently H, halo, alkyl, aryl or aralkyl;
(iii) R⁴ and R⁹, together with the atoms to which they are attached, form a 5-8 membered heterocyclic ring, R³ and R¹⁵ are each independently H, alkyl, aryl or aralkyl, and R⁵ to R⁸, R¹⁰ to R¹⁴, R¹⁶ and R¹⁷ are each independently H, halo, alkyl, aryl or aralkyl;
(iv) R³ and R⁷, together with the atoms to which they are attached, form a 5-8 membered heterocyclic ring, R⁴ and R⁹, together with the atoms to which they are attached, form a 5-8 membered heterocyclic ring, R¹⁵ is independently H, alkyl, aryl or aralkyl, and R⁵, R⁶, R⁸, R¹⁰ to R¹⁴, R¹⁶ and R¹⁷ are each independently H, halo, alkyl, aryl or aralkyl;
(v) R⁴ and R¹¹, together with the atoms to which they are attached, form a 3-8 membered heterocyclic ring, R³ and R¹⁵ are each independently H, alkyl, aryl or aralkyl, and R⁵ to R¹⁰, R¹² to R¹⁴, R¹⁶ and R¹⁷ are each independently H, halo, alkyl, aryl or aralkyl;
(vi) R¹¹and R¹², together with the atoms to which they are attached, form a 3-8 membered heterocyclic ring, R³, R⁴ and R¹⁵ are each independently H, alkyl, aryl or aralkyl, and R⁵ to R¹⁰, R¹³, R¹⁴, R¹⁶ and R¹⁷ are each independently H, halo, alkyl, aryl or aralkyl;
(vii) when X² is NR¹⁵, R¹¹ and R¹⁵, together with the atoms to which they are attached, form a 3-8 membered heterocyclic ring, R³ and R⁴ are each independently H, alkyl, aryl or aralkyl, and R⁵ to R¹⁰, R¹² to R¹⁴, R¹⁶ and R¹⁷ are each independently H, halo, alkyl, aryl or aralkyl;
(viii) R⁷ and R⁸, together with the atoms to which they are attached, form a 3-8 membered heterocyclic ring, R³, R⁴ and R¹⁵ are each independently H, alkyl, aryl or aralkyl, and R⁵, R⁶, R⁹ to R¹⁴, R¹⁶ and R¹⁷ are each independently H, halo, alkyl, aryl or aralkyl; or
(ix) when X¹ is NR¹⁵, R⁷ and R¹⁵, together with the atoms to which they are attached, form a 3-8 membered heterocyclic ring, R³ and R⁴ are each independently H, alkyl, aryl or aralkyl, and R⁵, R⁶, R⁸ to R¹⁴, R¹⁶ and R¹⁷ are each independently H, halo, alkyl, aryl or aralkyl;
with the proviso that the compound is not N-(1-(2-(methyl(2-(p-tolyloxy)ethyl)amino)-2-oxoethyl)-1H-pyrazol-4-yl)-3-phenoxypropanamide.

74. The method of claim 72 or claim 73, wherein the cardiovascular disease is myocardial infarction or atherosclerosis.

75. The method of claim 72 or claim 73, wherein the neurodegenerative disease is peripheral nerve injury, Creutzfeldt-Jakob disease, Parkinson's disease or Huntington's disease.

76. The method of claim 72 or claim 73, wherein the metabolic disorder is diabetes, type II diabetes or Gaucher disease.

77. The method of claim 72 or claim 73, wherein the hepatic disorder is non-alcoholic fatty liver disease (NAFLD).

78. The method of claim 72 or claim 73, wherein the protein misfolding disorder is an amyloid disease, Alzheimer's disease, an ocular disease, retinal degeneration, a lysosomal storage disease or alpha-1 antitrypsin deficiency, including alpha-1 antitrypsin associated emphysema and alpha-1 antitrypsin associated liver disease.

79. The method of claim 78, wherein the amyloid disease is atrial amyloidosis, spongiform encephalopathies, senile systemic amyloidosis, hereditary cerebral amyloid angiopathy, familial amyloid polyneuropathy I and II, or familial amyloidosis.

80. The method of claim 72 or claim 73, wherein the gastrointestinal disorder is Crohn's disease.

81. A method of treating retinitis pigmentosa, diabetic retinopathy or retinal neurodegeneration in a subject, comprising administering to the subject the compound of any one of claims 1-70 or the pharmaceutical composition of claim 71.

82. A method of treating retinitis pigmentosa, diabetic retinopathy or retinal neurodegeneration in a subject, comprising administering to the subject the compound of Formula I: 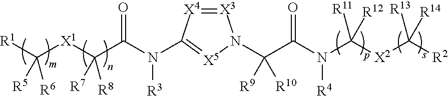
or a pharmaceutically acceptable derivative thereof, wherein:
m, n, p and s are each independently an integer from 0-4;
R¹ and R² are each independently aryl or heteroaryl;
X¹ and X² are each independently a bond, O or NR¹⁵;
X³ and X⁴ are each independently CR¹⁶ or N;
X⁵ is CR¹⁷ or N; and
R³ to R¹⁷ are selected from (i) to (ix):
(i) R³, R⁴ and R¹⁵ are each independently H, alkyl, aryl or aralkyl, and R⁵ to R¹⁴, R¹⁶ and R¹⁷ are each independently H, halo, alkyl, aryl or aralkyl;
(ii) R³ and R⁷, together with the atoms to which they are attached, form a 5-8 membered heterocyclic ring, R⁴ and R¹⁵ are each independently H, alkyl, aryl or aralkyl, and R⁵, R⁶, R⁸ to R¹⁴, R¹⁶ and R¹⁷ are each independently H, halo, alkyl, aryl or aralkyl;
(iii) R⁴ and R⁹, together with the atoms to which they are attached, form a 5-8 membered heterocyclic ring, R³ and R¹⁵ are each independently H, alkyl, aryl or aralkyl, and R⁵ to R⁸, R¹⁰ to R¹⁴, R¹⁶ and R¹⁷ are each independently H, halo, alkyl, aryl or aralkyl;
(iv) R³ and R⁷, together with the atoms to which they are attached, form a 5-8 membered heterocyclic ring, R⁴ and R⁹, together with the atoms to which they are attached, form a 5-8 membered heterocyclic ring, R¹⁵ is independently H, alkyl, aryl or aralkyl, and R⁵, R⁶, R⁸, R¹⁰ to R¹⁴, R¹⁶ and R¹⁷ are each independently H, halo, alkyl, aryl or aralkyl;
(v) R⁴ and R¹¹, together with the atoms to which they are attached, form a 3-8 membered heterocyclic ring, R³ and R¹⁵ are each independently H, alkyl, aryl or aralkyl, and R⁵ to R¹⁰, R¹² to R¹⁴, R¹⁶ and R¹⁷ are each independently H, halo, alkyl, aryl or aralkyl;
(vi) R¹¹ and R¹², together with the atoms to which they are attached, form a 3-8 membered heterocyclic ring, R³, R⁴ and R¹⁵ are each independently H, alkyl, aryl or aralkyl, and R⁵ to R¹⁰, R¹³, R¹⁴, R¹⁶ and R¹⁷ are each independently H, halo, alkyl, aryl or aralkyl;
(vii) when X² is NR¹⁵, R¹¹ and R¹⁵, together with the atoms to which they are attached, form a 3-8 membered heterocyclic ring, R³ and R⁴ are each independently H, alkyl, aryl or aralkyl, and R⁵ to R¹⁰, R¹² to R¹⁴, R¹⁶ and R¹⁷ are each independently H, halo, alkyl, aryl or aralkyl;
(viii) R⁷ and R⁸, together with the atoms to which they are attached, form a 3-8 membered heterocyclic ring, R³, R⁴ and R¹⁵ are each independently H, alkyl, aryl or aralkyl, and R⁵, R⁶, R⁹ to R¹⁴, R¹⁶ and R¹⁷ are each independently H, halo, alkyl, aryl or aralkyl; or
(ix) when X¹ is NR¹⁵, R⁷ and R¹⁵, together with the atoms to which they are attached, form a 3-8 membered heterocyclic ring, R³ and R⁴ are each independently H, alkyl, aryl or aralkyl, and R⁵, R⁶, R⁸ to R¹⁴, R¹⁶ and R¹⁷ are each independently H, halo, alkyl, aryl or aralkyl;
with the proviso that the compound is not N-(1-(2-(methyl(2-(p-tolyloxy)ethyl)amino)-2-oxoethyl)-1H-pyrazol-4-yl)-3-phenoxypropanamide.

83. A method of treating idiopathic epilepsy in a subject, comprising administering to the subject the compound of any one of claims 1-70 or the pharmaceutical composition of claim 71.

84. A method of treating idiopathic epilepsy in a subject, comprising administering to the subject the compound of Formula I: 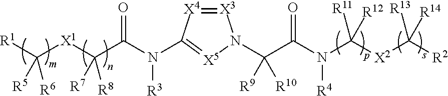
or a pharmaceutically acceptable derivative thereof, wherein:
m, n, p and s are each independently an integer from 0-4;
R¹ and R² are each independently aryl or heteroaryl;
X¹ and X² are each independently a bond, O or NR¹⁵;
X³ and X⁴ are each independently CR¹⁶ or N;
X⁵ is CR¹⁷ or N; and
R³ to R¹⁷ are selected from (i) to (ix):
(i) R³, R⁴ and R¹⁵ are each independently H, alkyl, aryl or aralkyl, and R⁵ to R¹⁴, R¹⁶ and R¹⁷ are each independently H, halo, alkyl, aryl or aralkyl;
(ii) R³ and R⁷, together with the atoms to which they are attached, form a 5-8 membered heterocyclic ring, R⁴ and R¹⁵ are each independently H, alkyl, aryl or aralkyl, and R⁵, R⁶, R⁸ to R¹⁴, R¹⁶ and R¹⁷ are each independently H, halo, alkyl, aryl or aralkyl;
(iii) R⁴ and R⁹, together with the atoms to which they are attached, form a 5-8 membered heterocyclic ring, R³ and R¹⁵ are each independently H, alkyl, aryl or aralkyl, and R⁵ to R⁸, R¹⁰ to R¹⁴, R¹⁶ and R¹⁷ are each independently H, halo, alkyl, aryl or aralkyl;
(iv) R³ and R⁷, together with the atoms to which they are attached, form a 5-8 membered heterocyclic ring, R⁴ and R⁹, together with the atoms to which they are attached, form a 5-8 membered heterocyclic ring, R¹⁵ is independently H, alkyl, aryl or aralkyl, and R⁵, R⁶, R⁸, R¹⁰ to R¹⁴, R¹⁶ and R¹⁷ are each independently H, halo, alkyl, aryl or aralkyl;
(v) R⁴ and R¹¹, together with the atoms to which they are attached, form a 3-8 membered heterocyclic ring, R³ and R¹⁵ are each independently H, alkyl, aryl or aralkyl, and R⁵ to R¹⁰, R¹² to R¹⁴, R¹⁶ and R¹⁷ are each independently H, halo, alkyl, aryl or aralkyl;
(vi) R¹¹ and R¹², together with the atoms to which they are attached, form a 3-8 membered heterocyclic ring, R³, R⁴ and R¹⁵ are each independently H, alkyl, aryl or aralkyl, and R⁵ to R¹⁰, R¹³, R¹⁴, R¹⁶ and R¹⁷ are each independently H, halo, alkyl, aryl or aralkyl;
(vii) when X² is NR¹⁵, R¹¹ and R¹⁵, together with the atoms to which they are attached, form a 3-8 membered heterocyclic ring, R³ and R⁴ are each independently H, alkyl, aryl or aralkyl, and R⁵ to R¹⁰, R¹² to R¹⁴, R¹⁶ and R¹⁷ are each independently H, halo, alkyl, aryl or aralkyl;
(viii) R⁷ and R⁸, together with the atoms to which they are attached, form a 3-8 membered heterocyclic ring, R³, R⁴ and R¹⁵ are each independently H, alkyl, aryl or aralkyl, and R⁵, R⁶, R⁹ to R¹⁴, R¹⁶ and R¹⁷ are each independently H, halo, alkyl, aryl or aralkyl; or
(ix) when X¹ is NR¹⁵, R⁷ and R¹⁵, together with the atoms to which they are attached, form a 3-8 membered heterocyclic ring, R³ and R⁴ are each independently H, alkyl, aryl or aralkyl, and R⁵, R⁶, R⁸ to R¹⁴, R¹⁶ and R¹⁷ are each independently H, halo, alkyl, aryl or aralkyl;
with the proviso that the compound is not N-(1-(2-(methyl(2-(p-tolyloxy)ethyl)amino)-2-oxoethyl)-1H-pyrazol-4-yl)-3-phenoxypropanamide.

85. A method of treating chondrodysplasia in a subject, comprising administering to the subject the compound of any one of claims 1-70 or the pharmaceutical composition of claim 71.

86. A method of treating chondrodysplasia in a subject, comprising administering to the subject the compound of Formula I: 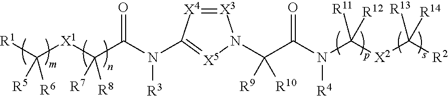
or a pharmaceutically acceptable derivative thereof, wherein:
m, n, p and s are each independently an integer from 0-4;
R¹ and R² are each independently aryl or heteroaryl;
X¹ and X² are each independently a bond, O or NR¹⁵;
X³ and X⁴ are each independently CR¹⁶ or N;
X⁵ is CR¹⁷ or N; and
R³ to R¹⁷ are selected from (i) to (ix):
(i) R³, R⁴ and R¹⁵ are each independently H, alkyl, aryl or aralkyl, and R⁵ to R¹⁴, R¹⁶ and R¹⁷ are each independently H, halo, alkyl, aryl or aralkyl;
(ii) R³ and R⁷, together with the atoms to which they are attached, form a 5-8 membered heterocyclic ring, R⁴ and R¹⁵ are each independently H, alkyl, aryl or aralkyl, and R⁵, R⁶, R⁸ to R¹⁴, R¹⁶ and R¹⁷ are each independently H, halo, alkyl, aryl or aralkyl;
(iii) R⁴ and R⁹, together with the atoms to which they are attached, form a 5-8 membered heterocyclic ring, R³ and R¹⁵ are each independently H, alkyl, aryl or aralkyl, and R⁵ to R⁸, R¹⁰ to R¹⁴, R¹⁶ and R¹⁷ are each independently H, halo, alkyl, aryl or aralkyl;
(iv) R³ and R⁷, together with the atoms to which they are attached, form a 5-8 membered heterocyclic ring, R⁴ and R⁹, together with the atoms to which they are attached, form a 5-8 membered heterocyclic ring, R¹⁵ is independently H, alkyl, aryl or aralkyl, and R⁵, R⁶, R⁸, R¹⁰ to R¹⁴, R¹⁶ and R¹⁷ are each independently H, halo, alkyl, aryl or aralkyl;
(v) R⁴ and R¹¹, together with the atoms to which they are attached, form a 3-8 membered heterocyclic ring, R³ and R¹⁵ are each independently H, alkyl, aryl or aralkyl, and R⁵ to R¹⁰, R¹² to R¹⁴, R¹⁶ and R¹⁷ are each independently H, halo, alkyl, aryl or aralkyl;
(vi) R¹¹ and R¹², together with the atoms to which they are attached, form a 3-8 membered heterocyclic ring, R³, R⁴ and R¹⁵ are each independently H, alkyl, aryl or aralkyl, and R⁵ to R¹⁰, R¹³, R¹⁴, R¹⁶ and R¹⁷ are each independently H, halo, alkyl, aryl or aralkyl;
(vii) when X² is NR¹⁵, R¹¹ and R¹⁵, together with the atoms to which they are attached, form a 3-8 membered heterocyclic ring, R³ and R⁴ are each independently H, alkyl, aryl or aralkyl, and R⁵ to R¹⁰, R¹² to R¹⁴, R¹⁶ and R¹⁷ are each independently H, halo, alkyl, aryl or aralkyl;
(viii) R⁷ and R⁸, together with the atoms to which they are attached, form a 3-8 membered heterocyclic ring, R³, R⁴ and R¹⁵ are each independently H, alkyl, aryl or aralkyl, and R⁵, R⁶, R⁹ to R¹⁴, R¹⁶ and R¹⁷ are each independently H, halo, alkyl, aryl or aralkyl; or
(ix) when X¹ is NR¹⁵, R⁷ and R¹⁵, together with the atoms to which they are attached, form a 3-8 membered heterocyclic ring, R³ and R⁴ are each independently H, alkyl, aryl or aralkyl, and R⁵, R⁶, R⁸ to R¹⁴, R¹⁶ and R¹⁷ are each independently H, halo, alkyl, aryl or aralkyl;
with the proviso that the compound is not N-(1-(2-(methyl(2-(p-tolyloxy)ethyl)amino)-2-oxoethyl)-1H-pyrazol-4-yl)-3-phenoxypropanamide.

87. A method of increasing IRE1 or XBP1s activity in a subject, comprising administering to the subject a compound of any one of claims 1-70 or a pharmaceutical composition of claim 71.

88. A method of increasing IRE1 or XBP1s activity in a subject, comprising administering to the subject a compound of Formula I: 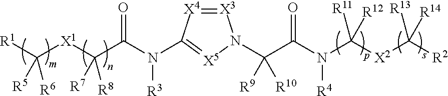
or a pharmaceutically acceptable derivative thereof, wherein:
m, n, p and s are each independently an integer from 0-4;
R¹ and R² are each independently aryl or heteroaryl;
X¹ and X² are each independently a bond, O or NR¹⁵;
X³ and X⁴ are each independently CR¹⁶ or N;
X⁵ is CR¹⁷ or N; and
R³ to R¹⁷ are selected from (i) to (ix):
(i) R³, R⁴ and R¹⁵ are each independently H, alkyl, aryl or aralkyl, and R⁵ to R¹⁴, R¹⁶ and R¹⁷ are each independently H, halo, alkyl, aryl or aralkyl;
(ii) R³ and R⁷, together with the atoms to which they are attached, form a 5-8 membered heterocyclic ring, R⁴ and R¹⁵ are each independently H, alkyl, aryl or aralkyl, and R⁵, R⁶, R⁸ to R¹⁴, R¹⁶ and R¹⁷ are each independently H, halo, alkyl, aryl or aralkyl;
(iii) R⁴ and R⁹, together with the atoms to which they are attached, form a 5-8 membered heterocyclic ring, R³ and R¹⁵ are each independently H, alkyl, aryl or aralkyl, and R⁵ to R⁸, R¹⁰ to R¹⁴, R¹⁶ and R¹⁷ are each independently H, halo, alkyl, aryl or aralkyl;
(iv) R³ and R⁷, together with the atoms to which they are attached, form a 5-8 membered heterocyclic ring, R⁴ and R⁹, together with the atoms to which they are attached, form a 5-8 membered heterocyclic ring, R¹⁵ is independently H, alkyl, aryl or aralkyl, and R⁵, R⁶, R⁸, R¹⁰ to R¹⁴, R¹⁶ and R¹⁷ are each independently H, halo, alkyl, aryl or aralkyl;
(v) R⁴ and R¹¹, together with the atoms to which they are attached, form a 3-8 membered heterocyclic ring, R³ and R¹⁵ are each independently H, alkyl, aryl or aralkyl, and R⁵ to R¹⁰, R¹² to R¹⁴, R¹⁶ and R¹⁷ are each independently H, halo, alkyl, aryl or aralkyl;
(vi) R¹¹ and R¹², together with the atoms to which they are attached, form a 3-8 membered heterocyclic ring, R³, R⁴ and R¹⁵ are each independently H, alkyl, aryl or aralkyl, and R⁵ to R¹⁰, R¹³, R¹⁴, R¹⁶ and R¹⁷ are each independently H, halo, alkyl, aryl or aralkyl;
(vii) when X² is NR¹⁵, R¹¹ and R¹⁵, together with the atoms to which they are attached, form a 3-8 membered heterocyclic ring, R³ and R⁴ are each independently H, alkyl, aryl or aralkyl, and R⁵ to R¹⁰, R¹² to R¹⁴, R¹⁶ and R¹⁷ are each independently H, halo, alkyl, aryl or aralkyl;
(viii) R⁷ and R⁸, together with the atoms to which they are attached, form a 3-8 membered heterocyclic ring, R³, R⁴ and R¹⁵ are each independently H, alkyl, aryl or aralkyl, and R⁵, R⁶, R⁹ to R¹⁴, R¹⁶ and R¹⁷ are each independently H, halo, alkyl, aryl or aralkyl; or
(ix) when X¹ is NR¹⁵, R⁷ and R¹⁵, together with the atoms to which they are attached, form a 3-8 membered heterocyclic ring, R³ and R⁴ are each independently H, alkyl, aryl or aralkyl, and R⁵, R⁶, R⁸ to R¹⁴, R¹⁶ and R¹⁷ are each independently H, halo, alkyl, aryl or aralkyl;
with the proviso that the compound is not N-(1-(2-(methyl(2-(p-tolyloxy)ethyl)amino)-2-oxoethyl)-1H-pyrazol-4-yl)-3-phenoxypropanamide.

89. A method of treating idiopathic epilepsy, Dravet syndrome or Lennox-Gastaut syndrome in a subject, comprising administering to the subject the compound of any one of claims 1-70 or the pharmaceutical composition of claim 71.

90. A method of treating idiopathic epilepsy, Dravet syndrome or Lennox-Gastaut syndrome in a subject, comprising administering to the subject a compound of Formula I: 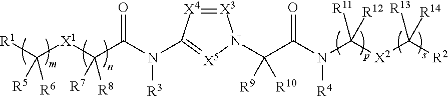
or a pharmaceutically acceptable derivative thereof, wherein:
m, n, p and s are each independently an integer from 0-4;
R¹ and R² are each independently aryl or heteroaryl;
X¹ and X² are each independently a bond, O or NR¹⁵;
X³ and X⁴ are each independently CR¹⁶ or N;
X⁵ is CR¹⁷ or N; and
R³ to R¹⁷ are selected from (i) to (ix):
(i) R³, R⁴ and R¹⁵ are each independently H, alkyl, aryl or aralkyl, and R⁵ to R¹⁴, R¹⁶ and R¹⁷ are each independently H, halo, alkyl, aryl or aralkyl;
(ii) R³ and R⁷, together with the atoms to which they are attached, form a 5-8 membered heterocyclic ring, R⁴ and R¹⁵ are each independently H, alkyl, aryl or aralkyl, and R⁵, R⁶, R⁸ to R¹⁴, R¹⁶ and R¹⁷ are each independently H, halo, alkyl, aryl or aralkyl;
(iii) R⁴ and R⁹, together with the atoms to which they are attached, form a 5-8 membered heterocyclic ring, R³ and R¹⁵ are each independently H, alkyl, aryl or aralkyl, and R⁵ to R⁸, R¹⁰ to R¹⁴, R¹⁶ and R¹⁷ are each independently H, halo, alkyl, aryl or aralkyl;
(iv) R³ and R⁷, together with the atoms to which they are attached, form a 5-8 membered heterocyclic ring, R⁴ and R⁹, together with the atoms to which they are attached, form a 5-8 membered heterocyclic ring, R¹⁵ is independently H, alkyl, aryl or aralkyl, and R⁵, R⁶, R⁸, R¹⁰ to R¹⁴, R¹⁶ and R¹⁷ are each independently H, halo, alkyl, aryl or aralkyl;
(v) R⁴ and R¹¹, together with the atoms to which they are attached, form a 3-8 membered heterocyclic ring, R³ and R¹⁵ are each independently H, alkyl, aryl or aralkyl, and R⁵ to R¹⁰, R¹² to R¹⁴, R¹⁶ and R¹⁷ are each independently H, halo, alkyl, aryl or aralkyl;
(vi) R¹¹ and R¹², together with the atoms to which they are attached, form a 3-8 membered heterocyclic ring, R³, R⁴ and R¹⁵ are each independently H, alkyl, aryl or aralkyl, and R⁵ to R¹⁰, R¹³, R¹⁴, R¹⁶ and R¹⁷ are each independently H, halo, alkyl, aryl or aralkyl;
(vii) when X² is NR¹⁵, R¹¹ and R¹⁵, together with the atoms to which they are attached, form a 3-8 membered heterocyclic ring, R³ and R⁴ are each independently H, alkyl, aryl or aralkyl, and R⁵ to R¹⁰, R¹² to R¹⁴, R¹⁶ and R¹⁷ are each independently H, halo, alkyl, aryl or aralkyl;
(viii) R⁷ and R⁸, together with the atoms to which they are attached, form a 3-8 membered heterocyclic ring, R³, R⁴ and R¹⁵ are each independently H, alkyl, aryl or aralkyl, and R⁵, R⁶, R⁹ to R¹⁴, R¹⁶ and R¹⁷ are each independently H, halo, alkyl, aryl or aralkyl; or
(ix) when X¹ is NR¹⁵, R⁷ and R¹⁵, together with the atoms to which they are attached, form a 3-8 membered heterocyclic ring, R³ and R⁴ are each independently H, alkyl, aryl or aralkyl, and R⁵, R⁶, R⁸ to R¹⁴, R¹⁶ and R¹⁷ are each independently H, halo, alkyl, aryl or aralkyl;
with the proviso that the compound is not N-(1-(2-(methyl(2-(p-tolyloxy)ethyl)amino)-2-oxoethyl)-1H-pyrazol-4-yl)-3-phenoxypropanamide.

91. A method of treating genetic epilepsy in a subject, comprising administering to the subject the compound of any one of claims 1-70 or the pharmaceutical composition of claim 71.

92. A method of treating genetic epilepsy in a subject, comprising administering to the subject a compound of Formula I: 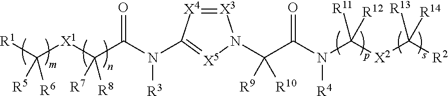
or a pharmaceutically acceptable derivative thereof, wherein:
m, n, p and s are each independently an integer from 0-4;
R¹ and R² are each independently aryl or heteroaryl;
X¹ and X² are each independently a bond, O or NR¹⁵;
X³ and X⁴ are each independently CR¹⁶ or N;
X⁵ is CR¹⁷ or N; and
R³ to R¹⁷ are selected from (i) to (ix):
(i) R³, R⁴ and R¹⁵ are each independently H, alkyl, aryl or aralkyl, and R⁵ to R¹⁴, R¹⁶ and R¹⁷ are each independently H, halo, alkyl, aryl or aralkyl;
(ii) R³ and R⁷, together with the atoms to which they are attached, form a 5-8 membered heterocyclic ring, R⁴ and R¹⁵ are each independently H, alkyl, aryl or aralkyl, and R⁵, R⁶, R⁸ to R¹⁴, R¹⁶ and R¹⁷ are each independently H, halo, alkyl, aryl or aralkyl;
(iii) R⁴ and R⁹, together with the atoms to which they are attached, form a 5-8 membered heterocyclic ring, R³ and R¹⁵ are each independently H, alkyl, aryl or aralkyl, and R⁵ to R⁸, R¹⁰ to R¹⁴, R¹⁶ and R¹⁷ are each independently H, halo, alkyl, aryl or aralkyl;
(iv) R³ and R⁷, together with the atoms to which they are attached, form a 5-8 membered heterocyclic ring, R⁴ and R⁹, together with the atoms to which they are attached, form a 5-8 membered heterocyclic ring, R¹⁵ is independently H, alkyl, aryl or aralkyl, and R⁵, R⁶, R⁸, R¹⁰ to R¹⁴, R¹⁶ and R¹⁷ are each independently H, halo, alkyl, aryl or aralkyl;
(v) R⁴ and R¹¹, together with the atoms to which they are attached, form a 3-8 membered heterocyclic ring, R³ and R¹⁵ are each independently H, alkyl, aryl or aralkyl, and R⁵ to R¹⁰, R¹² to R¹⁴, R¹⁶ and R¹⁷ are each independently H, halo, alkyl, aryl or aralkyl;
(vi) R¹¹ and R¹², together with the atoms to which they are attached, form a 3-8 membered heterocyclic ring, R³, R⁴ and R¹⁵ are each independently H, alkyl, aryl or aralkyl, and R⁵ to R¹⁰, R¹³, R¹⁴, R¹⁶ and R¹⁷ are each independently H, halo, alkyl, aryl or aralkyl;
(vii) when X² is NR¹⁵, R¹¹ and R¹⁵, together with the atoms to which they are attached, form a 3-8 membered heterocyclic ring, R³ and R⁴ are each independently H, alkyl, aryl or aralkyl, and R⁵ to R¹⁰, R¹² to R¹⁴, R¹⁶ and R¹⁷ are each independently H, halo, alkyl, aryl or aralkyl;
(viii) R⁷ and R⁸, together with the atoms to which they are attached, form a 3-8 membered heterocyclic ring, R³, R⁴ and R¹⁵ are each independently H, alkyl, aryl or aralkyl, and R⁵, R⁶, R⁹ to R¹⁴, R¹⁶ and R¹⁷ are each independently H, halo, alkyl, aryl or aralkyl; or
(ix) when X¹ is NR¹⁵, R⁷ and R¹⁵, together with the atoms to which they are attached, form a 3-8 membered heterocyclic ring, R³ and R⁴ are each independently H, alkyl, aryl or aralkyl, and R⁵, R⁶, R⁸ to R¹⁴, R¹⁶ and R¹⁷ are each independently H, halo, alkyl, aryl or aralkyl;
with the proviso that the compound is not N-(1-(2-(methyl(2-(p-tolyloxy)ethyl)amino)-2-oxoethyl)-1H-pyrazol-4-yl)-3-phenoxypropanamide.

93. A method of treating genetic epilepsy due to at least one variant in GABAA receptor subunits in a subject, comprising administering to the subject the compound of any one of claims 1-70 or the pharmaceutical composition of claim 71.

94. A method of treating genetic epilepsy due to at least one variant in GABAA receptor subunits in a subject, comprising administering to the subject a compound of Formula I: 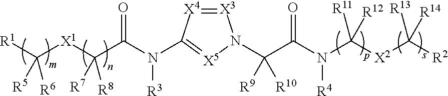
or a pharmaceutically acceptable derivative thereof, wherein:
m, n, p and s are each independently an integer from 0-4;
R¹ and R² are each independently aryl or heteroaryl;
X¹ and X² are each independently a bond, O or NR¹⁵;
X³ and X⁴ are each independently CR¹⁶ or N;
X⁵ is CR¹⁷ or N; and
R³ to R¹⁷ are selected from (i) to (ix):
(i) R³, R⁴ and R¹⁵ are each independently H, alkyl, aryl or aralkyl, and R⁵ to R¹⁴, R¹⁶ and R¹⁷ are each independently H, halo, alkyl, aryl or aralkyl;
(ii) R³ and R⁷, together with the atoms to which they are attached, form a 5-8 membered heterocyclic ring, R⁴ and R¹⁵ are each independently H, alkyl, aryl or aralkyl, and R⁵, R⁶, R⁸ to R¹⁴, R¹⁶ and R¹⁷ are each independently H, halo, alkyl, aryl or aralkyl;
(iii) R⁴ and R⁹, together with the atoms to which they are attached, form a 5-8 membered heterocyclic ring, R³ and R¹⁵ are each independently H, alkyl, aryl or aralkyl, and R⁵ to R⁸, R¹⁰ to R¹⁴, R¹⁶ and R¹⁷ are each independently H, halo, alkyl, aryl or aralkyl;
(iv) R³ and R⁷, together with the atoms to which they are attached, form a 5-8 membered heterocyclic ring, R⁴ and R⁹, together with the atoms to which they are attached, form a 5-8 membered heterocyclic ring, R¹⁵ is independently H, alkyl, aryl or aralkyl, and R⁵, R⁶, R⁸, R¹⁰ to R¹⁴, R¹⁶ and R¹⁷ are each independently H, halo, alkyl, aryl or aralkyl;
(v) R⁴ and R¹¹, together with the atoms to which they are attached, form a 3-8 membered heterocyclic ring, R³ and R¹⁵ are each independently H, alkyl, aryl or aralkyl, and R⁵ to R¹⁰, R¹² to R¹⁴, R¹⁶ and R¹⁷ are each independently H, halo, alkyl, aryl or aralkyl;
(vi) R¹¹ and R¹², together with the atoms to which they are attached, form a 3-8 membered heterocyclic ring, R³, R⁴ and R¹⁵ are each independently H, alkyl, aryl or aralkyl, and R⁵ to R¹⁰, R¹³, R¹⁴, R¹⁶ and R¹⁷ are each independently H, halo, alkyl, aryl or aralkyl;
(vii) when X² is NR¹⁵, R¹¹ and R¹⁵, together with the atoms to which they are attached, form a 3-8 membered heterocyclic ring, R³ and R⁴ are each independently H, alkyl, aryl or aralkyl, and R⁵ to R¹⁰, R¹² to R¹⁴, R¹⁶ and R¹⁷ are each independently H, halo, alkyl, aryl or aralkyl;
(viii) R⁷ and R⁸, together with the atoms to which they are attached, form a 3-8 membered heterocyclic ring, R³, R⁴ and R¹⁵ are each independently H, alkyl, aryl or aralkyl, and R⁵, R⁶, R⁹ to R¹⁴, R¹⁶ and R¹⁷ are each independently H, halo, alkyl, aryl or aralkyl; or
(ix) when X¹ is NR¹⁵, R⁷ and R¹⁵, together with the atoms to which they are attached, form a 3-8 membered heterocyclic ring, R³ and R⁴ are each independently H, alkyl, aryl or aralkyl, and R⁵, R⁶, R⁸ to R¹⁴, R¹⁶ and R¹⁷ are each independently H, halo, alkyl, aryl or aralkyl;
with the proviso that the compound is not N-(1-(2-(methyl(2-(p-tolyloxy)ethyl)amino)-2-oxoethyl)-1H-pyrazol-4-yl)-3-phenoxypropanamide.
